(19) United States
(12) Patent Application Publication
(10) Pub. No.: US 2023/0135127 A1
(43) Pub. Date: May 4, 2023

(54) COMPOSITIONS AND METHODS FOR DISEASE TREATMENT AND PREVENTION BY PH MODIFIERS AND/OR CELL PROLIFERATION INHIBITORS

(71) Applicant: Wei-ping Zeng, Huntington, WV (US)

(72) Inventor: Wei-ping Zeng, Huntington, WV (US)

(21) Appl. No.: 17/513,658

(22) Filed: Oct. 28, 2021

(51) Int. Cl. A61K 31/19; A61K 33/00; A61P 11/06; A61P 35/00; A61P 7/06; A61P 3/04

(52) U.S. Cl. CPC A61K 31/19; A61K 33/00; A61P 11/06; A61P 35/00; A61P 7/06; A61P 3/04

(57) ABSTRACT

The invention is partly based on the unexpected discoveries of cellular differences in response to pH alterations, and a key role of the cells' proliferative statuses in determining their responses. Thus, pH modifiers, their doses and dosing regimens are chosen to selectively deplete and/or suppress inflammatory and/or non-inflammatory pathological cells and their proliferation while preserving normal cells in diseases whose pathogenesis is partially or fully attributable to inflammation and/or cell proliferation. Conversely, pH modifiers, their doses and dosing regimens were chosen to selectively increase the population and/or proliferation of normal cells to promote protective immune and inflammatory responses, normal cell regeneration or renewal. For the treatment of overweight or obesity, pH modifiers are selected, and cell proliferation inhibitors are repurposed, to suppress epithelial renewal in the intestinal villi, or alternatively the small intestines are surgically shortened to reduce body weight and fat mass.

### TECHNICAL FIELD

The presently disclosed subject matter is directed to compositions and methods for use in the treatment and prevention of diseases, especially but not exclusively those whose pathogenesis is in part or in full attributable to inflammation, cell proliferation, or both, by using pH modifiers and/or cell proliferation inhibitors.

### BACKGROUND

Inflammation is a necessary process for wound healing, the resolution of infection, and establishment of immunity against infections and tumors. However, inflammation may also impair the structures and functions of tissues and organs in a variety of diseases, such as (but not limited to) allergic, autoimmune, and infectious diseases, and/or atherosclerotic cardiovascular diseases (ASCVD). Both the innate and adaptive arms of the immune system contribute to inflammation in intricately interactive manners. The innate immune cells are often the first responders to various triggers of inflammation. The activation of innate immune cells by a variety of molecules (e.g., the pathogen associated molecular patterns (PAMPs) and the danger-associated molecular patterns (DAMPs)) facilitates the transportation of antigens to draining lymph nodes and subsequent presentation of the antigens to the adaptive immune cells. (1, 2). Upon activation by specific antigens, T and B cells of the adaptive immune system differentiate into effector T cells and high affinity antibody-producing cells. The antibodies and effector T cells in turn dictate what types of innate immune cells are recruited to and activated at the site of inflammation upon re-encounter of the same triggers. For example, in allergic asthma, Th2 cells activated by allergens produce IL-5. IL-5 together with eotaxin are responsible for recruiting eosinophils to the lungs, which is a hallmark of allergic asthma. (3). On the other hand, cross-linking of IgE receptors on mast cells by IgE-allergen complex activates the mast cells to release histamine, a key mediator of the pathophysiology in allergic asthma. (4).

Apart from the accumulation of inflammatory cells, cell proliferation is another prominent and functionally important feature of inflammation. The adaptive immune cells such as the T cells and B cells undergo clonal expansion upon activation by specific antigens. In addition, inflammatory signals such as hypercholesterolemia or inflammatory mediators stimulate the progenitor cells of the innate immune cells to undergo accelerated proliferation and differentiation in the bone marrow and spleen to produce more innate immune cells. (5). Furthermore, the structural functional cells of the inflamed tissue may undergo hyperplasia that contributes to the pathogenesis of an inflammatory disease. For example, hyperplasia of pneumocytes, fibroblasts and goblet cells is common in pulmonary inflammation caused by infections or allergen exposures. (6, 7). Therefore, it would be desirable to have anti-inflammatory medicines that can directly deplete inflammatory and proliferating cells simultaneously. However, no such anti-inflammatory medicines are available.

Currently, the mainstream strategy of developing next generation of anti-inflammatory medicines focuses on blocking the expression or signaling of an inflammatory cytokine important for a specific disease. This strategy does not directly address cell proliferation and is often unsatisfactory in terms of therapeutic and cost effectiveness, while it also faces the hurdle of serious adverse side effects. Furthermore, despite many promising leads, examples of clinical success of this strategy remain scarce. (8, 9). Even if successfully developed, this kind of medicines is effective only for the specifically targeted diseases and is often cost inhibitory for many patients.

On the other hand, currently available broad-spectrum anti-inflammatory medicines glucocorticoids (GCs) and non-steroid anti-inflammatory drugs (NSAIDs) also have serious limitations. GCs function by suppressing the expression of pro-inflammatory cytokines and/or chemokines while inducing the expression of anti-inflammatory genes. (10, 11). NSAIDs are inhibitors of cyclooxygenases (COX), primarily COX-2. COXs mediate the synthesis of the pro-inflammatory mediators prostaglandins, particularly PGE2 and PG12. (12). A major limitation of GCs is resistance. (13). For example, most patients of chronic obstructive pulmonary disease (COPD) respond poorly to GCs, except very modest reduction of exacerbation. (14, 15). Likewise, only about 50% of clinical asthma is characterized as Th2-high endotype, less than half of which respond to GCs, whereas Th2-low and other endotypes are generally resistant. (16-20). Resistance to NSAID by various mechanisms is also well documented. (21). The use of GCs and/or NSAIDs is further limited by adverse side effects, such as immune suppression, bone and muscle loss, hyperglycemia, and weight gain. The use of NSAIDs is also associated with increased risk of myocardial infarction and stroke, erectile dysfunction, gastric ulceration, and chronic kidney disease. (13, 22, 23).

Adverse side effects are even of greater concerns in cancer chemotherapies and radiation therapies than in inflammatory diseases. In severe cases, the side effects can be life-threatening. For example, such therapies can lead to near complete destruction of the bone marrow stem cells, resulting in immunosuppression, myelosuppression and anemia. (24). In addition, tumor cell resistance to chemotherapy is common. (25). Unfortunately, although cancer patients have to endure such adverse side effects thereby drastic deterioration of quality of life, the effectiveness of the chemotherapies and radiation therapies in eradicating cancer cells can be disappointing. Thus, similar to therapeutic research for inflammatory diseases, current trend of developing new cancer therapies is to target specific molecules as antigens for cell-based immune therapies or to block signaling pathways for tumor cell growth or check points of immune suppression. While these approaches are advocated as having great promises, success is sporadic, and many scientific and technical obstacles remain. Such new drugs or therapies are also expected to be cost inhibitory for a large portion (if not the majority) of cancer patients, and of limited availabilities. Therefore, a novel and unconventional strategy for developing inexpensive and widely accessible therapies would be of great value to cancer patients.

Treatment of obesity faces similar challenges. Obesity is a great health problem of global scale. Currently there are a small number of drugs available for pharmacotherapy of obesity, all of which target food/nutrient intake. In general, these drugs are not well received by patients because their effects are rather modest and patients often regain body weight after the termination of the medication (26). Therefore, new therapies based on novel strategies are equally in great need.

Thus, it is of great clinical significance to explore a topic that has so far largely been neglected by the biomedical community—the potential different responses of different cells to pH fluctuation and their medical implications. The pH value in a human subject is maintained in a relatively narrow range from 7.35 to 7.45 with the average of 7.40. (27). For this reason, most cellular studies do not treat pH as a variable, instead are conducted in buffers or culture media that maintain a constant, near neutral pH.

However, in real life, an individual can experience transient systemic and local pH fluctuation without notable adverse clinical consequences. For example, at the systemic level, strenuous physical exercises and grand mal seizures can cause severe lactic acidosis without serious pathophysiological consequences in the aftermath. (28, 29). Fluctuation of pH also occurs locally at specific tissues and/or organs. For example, the blood vessels can stand considerable swings of pH in either direction. (28, 29). In the lungs, the air surface liquid (ASL) and alveolar subphase fluid (AVSF) have pH values around 6.6 and 6.9, respectively. (30, 31). The urine pH can range from 4.5 to 8. (32). However, although these rather sporadic reports have been published over a long period of time, there has been no investigation at the cellular level how different cells might respond differently to pH fluctuation, specifically, how inflammatory cells and the structural functional cells of a tissue might respond differently; and how proliferating and non-proliferating cells might respond differently. From the perspective of disease treatment and prevention, if such cellular differences exist, the important ensuing questions would be what medical implications are associated with such differences, and whether the body's tolerance to transient systemic or local pH fluctuation can be exploited for medical benefits. In this regard, the present invention is the first to investigate the cellular differences in responses to pH alterations and to design therapeutic and preventive regimens based in part on such differences.

The present invention offers several key advantages over other approaches to the treatment and/or prevention of diseases associated with inflammation and/or cell proliferation. For inflammatory and/or neoplastic diseases, the present invention provides means to simultaneously control inflammatory and proliferating cells. The means are broad spectrum, unlimited to the target cells' origins of tissues and organs. The present invention also offers a novel strategy for pharmacotherapies of overweight and obesity by targeting epithelial renewal in the intestines. The simplicity of the present invention promises cost effectiveness and availability to large patient populations including economically disadvantaged patients.

### SUMMARY

The present invention is based in part on the novel finding of cellular differences in response to pH alterations. Specifically, the Examples disclosed in this invention demonstrated that inflammatory cells are more susceptible to depletion by pH modifiers, especially but not exclusively those that decrease or resist the rise of pH, than tissue structural functional cells; and there is a positive correlation between a cell's proliferative status and the susceptibility of the cell and its proliferation to depletion or suppression by the pH modifiers. On the other hand, cell proliferation is positively correlated with pH. Treatments with pH modifiers, especially but not exclusively those that increase pH or resist the fall of pH, maintain or enhance cell proliferation. Thus, many embodiments of the present invention use pH modifiers to control the population of inflammatory cells, proliferating cells, or both. The invention therefore provides compositions and methods for the treatment and prevention of diseases whose pathogenesis is partially or fully attributable to inflammation, immune response, cell proliferation, or combinations thereof; and to treat overweight or obesity by using pH modifiers and/or cell proliferation inhibitors to reduce intestinal epithelial renewal.

In some embodiments, the presently disclosed subject matter is directed to compositions and method for selectively depleting, selectively suppressing, or both selectively deleting and suppressing the population, proliferation or both the population and proliferation of pathological cells in a subject. The subject is in need of treatment of, prevention from, or both treatment of and prevention from, a disease with a pathogenesis that is partially or fully attributable to inflammation, immune response, cell proliferation, or combinations thereof. The disclosed method comprises administering a composition comprising one or more pH modifiers that decrease pH, resist the rise of pH, or both, to the subject. Alternatively, the method comprises administering a composition comprising one or more pH modifiers that increase pH, resist the fall of pH, or both, to the subject. The administering step selectively depletes, selectively suppresses, or both selectively depletes and selectively suppresses the population, proliferation, or both of pathological cells in the subject.

In some embodiments, the compositions and method further comprise choosing the one or more pH modifiers, and routes and means of administration; titrating doses, dosing regimens, or both the doses and dosing regiments of the one or more pH modifiers, and titrating the concentrations of the one or more pH modifiers in the composition; or both the said choosing and titrating so that the pH modifiers selectively act on pathological cells.

In some embodiments, the disease is selected from one or more allergic diseases; autoimmune diseases; infectious diseases; inflammatory diseases of the blood, blood vessels, or both; diseases with pulmonary inflammation; muco-obstructive lung diseases; and other diseases sharing the characteristic of overzealous inflammatory responses, immune responses, or both that contribute to the pathogenesis, wherein the step of administrating dampens the overzealous inflammatory responses, immune responses, or both.

In some embodiments, the pathological cells comprise effector inflammatory cells; hyperplastic, hyperactive structural functional cells of diseased tissues or organs; host target cells; or combinations thereof.

In some embodiments, the disease is selected from one or more of neoplastic diseases and infectious diseases where lacking or insufficiency of protective inflammatory responses, immune responses, or both against neoplastic cells or infectious agents contributes to pathogenesis; wherein the step of administering promotes the protective inflammatory responses, immune responses, or both; and clearance of neoplastic cells or infections. In some embodiments, the pathological cells comprise neoplastic cells; functional subsets of inflammatory cells with anti-inflammatory, immune suppressive activities, or both; non-inflammatory tumor-promoting cells; hyperplastic, hyperactive structural functional cells of diseased tissues or organs; host target cells; or combinations thereof.

In some embodiments, the step of administering suppresses mucus hypersecretion in asthma or muco-obstructive lung diseases where mucus hypersecretion obstructs airflow, impairs respiratory function, or both.

In some embodiments, the step of administering the pH modifier(s) depletes, suppresses, or both depletes and suppresses population, proliferation, or both the population and proliferation, and dissemination of infectious agents in or on the body of the subject.

In some embodiments, wherein the subject is a recipient of vaccination, the step of administering selectively depletes and suppresses population, proliferation, or both the population and proliferation, of one or more of inflammatory cell functional subsets that undermine the efficacy or protective effects of the vaccination against one or more diseases selected from infectious diseases, neoplastic diseases, allergic diseases, and drug addictions.

In some embodiments, the presently disclosed subject matter is directed to compositions and method for selectively increasing population, proliferation, or both the population and proliferation of protective normal cells against a disease in a subject. The subject is in need of treatment of, prevention from, or both treatment of and prevention from the disease with a pathogenesis that is partially or fully attributable to inflammation, immune response, cell proliferation, or combinations thereof. The method comprises administering a composition comprising one or more pH modifiers that increase pH, resist the fall of pH or both to the subject, wherein the step of administering selectively increases the population, proliferation, or both, of the protective normal cells against a disease in a subject.

In some embodiments, the compositions and method further comprise choosing the one or more pH modifiers, and routes and means of administration; titrating doses, dosing regimens, or both the doses and dosing regimens of the one or more pH modifiers, titrating concentrations of the one or more pH modifiers in the composition; or both the said choosing and titrating so that the pH modifiers selectively act on the protective normal cells.

In some embodiments, the disease is selected from one or more of neoplastic diseases and infectious diseases where lacking or insufficiency of protective inflammatory responses, immune responses, or both against the neoplastic cells or infectious agents contributes to pathogenesis; wherein the step of administering promotes the protective inflammatory responses, immune responses, or both; and clearance of neoplastic cells or infections. In some embodiments, the protective normal cells are effector inflammatory cells directly or indirectly reactive to the neoplastic cells or infectious agents, and such cells' precursors; normal structural functional cells of diseased tissues or organs, and their precursors; or combinations thereof.

In some embodiments, the disease is selected from one or more allergic diseases; autoimmune diseases; infectious diseases; inflammatory diseases of blood, blood vessels, or both; diseases with pulmonary inflammation; muco-obstructive lung diseases; and other diseases sharing the characteristic of overzealous inflammatory responses, immune responses, or both that contribute to pathogenesis; wherein the step of administering dampens the overzealous inflammatory responses, immune responses, or both, and promotes restoration of damaged tissues or organs. The protective normal cells comprise functional subsets of inflammatory cells with anti-inflammatory, immune suppressive activities, or both; normal structural functional cells of tissues or organs, and their precursors; or combinations thereof.

In some embodiments, the subject is a recipient of vaccination, the step of administering enhances the efficacy or protective effects of the vaccination against one or more diseases selected from infectious diseases, neoplastic diseases, allergic diseases, and drug addictions. In the some embodiments, the protective normal cells in the vaccine recipient are one or more functional subsets of inflammatory cells directly or indirectly reactive to infectious agents, neoplastic cells, or one or more addictive drugs or molecules involved in the drugs' actions.

In some embodiments, the subject is an autologous or allogeneic donor of blood, bone marrow, or stem cells for adoptive cell transfer; or suffers from anemia or insufficient genesis of lymphocytes and hematopoietic cells; and wherein the step of administering selectively increases the population, proliferation, or both the population and proliferation of lymphocytes, hematopoietic cells, the precursors of lymphocytes and hematopoietic cells, other stem cells, or combinations thereof.

In some embodiments, the subject suffers from one or more wounds, and wherein the step of administering facilitates wound healing.

In some embodiments, the step of administering the pH modifier(s) depletes, suppresses, or both depletes and suppresses population, proliferation, or both the population and proliferation, and dissemination of infectious agents in or on the body of the subject.

In some embodiments, the presently disclosed subject matter is directed to composition and methods for reducing body weight, fat mass, or both body weight and fat mass in a subject who suffers from overweight or obesity.

In some embodiments, the compositions and method further comprise administering a composition to the subject, wherein the step of administering selectively reduce population, proliferation, or both the population and proliferation of proliferating cells in epithelium of intestinal villi of the subject; and wherein the composition comprises one or more pH modifiers or cell proliferation inhibitors; or combinations of one or more of pH modifiers and cell proliferation inhibitors. The step of administering further comprises choosing the one or more pH modifiers or cell proliferation inhibitors, or both, and routes and means of administration; titrating doses, dosing regimens, or both the doses and dosing regimens of the one or more pH modifiers or cell proliferation inhibitors, and titrating the concentrations of the one or more pH modifiers and/or cell proliferation inhibitors in the composition; or both the said choosing and titrating; to achieve selective effects on proliferating cells in the epithelium of the intestinal villi of the subject.

In some embodiments, the method further comprises surgically shortening small intestines of the subject.

### BRIEF DESCRIPTION OF THE DRAWINGS

FIGS. 1a-1d are microscopic views of hematoxylin and eosin (H&E)-stained cells in the bronchoalveolar lavage fluids (BALF) from Balb/c mice sensitized and challenged with ovalbumin (OVA), and treated with saline, or saline plus HCl, HOAc, or NaOH, respectively.

FIG. 1e is a bar graph illustrating the averages of total numbers of cells per mouse in the BALF of mice represented in FIGS. 1a-d. Error bars are standard deviations. Statistical significance of the differences between saline and other treatment groups was determined by Student t test; * (p<0.05); ** (p<0.01).

FIGS. 1f-1h are bar graphs illustrating the average percentages of eosinophils, macrophages, and lymphocytes in the BALF cells.

FIG. 2a shows flow cytometric pseudocolor plots of blood samples from mice described in FIG. 1, illustrating the populations of granulocytes and lymphocytes.

FIG. 2b is a bar graph illustrating the percentage of granulocytes and lymphocytes in the RBC-depleted blood samples of the same mice.

FIGS. 3a-3d are microscopic views at low magnification of the H&E-stained lung tissues of mice in the different treatment groups as in FIG. 1: saline (FIG. 3a), HCl (FIG. 3b), HOAc (FIG. 3c), or NaOH (FIG. 3d). Blood vessels (BV) and bronchi/bronchioles (Br) are indicated.

FIGS. 3e-3h are microscopic views at high magnification of the H&E-stained lung tissues of mice in the different treatment groups as in FIG. 1.

FIGS. 4a-4d are microscopic views of PAS-stained lung tissues from mice of the different treatment groups as in FIG. 1: saline (FIG. 4a), HCl (FIG. 4b), HOAc (FIG. 4c), or NaOH (FIG. 4d).

FIG. 5a shows pseudocolor plots illustrating the expression of the T cell activation markers CD44 and CD69 in the Foxp3⁻ CD4 T cells in the BALF (upper panels) and mediastinal lymph nodes (MLNs) (lower panels) from mice of the different treatment groups as in FIG. 1.

FIGS. 5b (BALF) and 5c (MLN) are bar graphs based on FIG. 5a, illustrating the percentages of CD44⁺ and CD44⁺CD69⁺ cells in the Foxp⁻ CD4 T cells.

FIG. 5d shows pseudocolor plots illustrating the expression of Foxp3 and CD4 by BALF (upper panels) and MLN (lower panels) cells of mice of the different treatment groups as in FIG. 1.

FIGS. 5e and 5f are bar graphs based on FIG. 5d, illustrating the percentages of the Foxp3⁺ Treg cells in total CD4 T cells.

FIG. 6a is a pseudocolor plot showing the gates of lymphocyte (Lym) and granulocyte (Gran) populations in the BALF cells of BALB/c mice sensitized and challenged with OVA.

FIG. 6b is a pseudocolor plot showing the gates of eosinophils (Eos) and macrophages (Mac) within the granulocyte population of FIG. 6a based on their expression of F4/80 and Siglec F.

FIG. 6c is a set of pseudocolor plots showing the profiles of Annexin V and 7AAd staining in lymphocyte, eosinophil, and macrophage populations defined by the gates in FIG. 6a and FIG. 6b in the BALF from BALB/c mice sensitized with OVA, challenged with OVA, and treated with saline or saline plus HOAc. Numbers in the plots are percentages of the cell populations in their respective quadrants.

FIG. 6d is a pseudocolor plot of peripheral blood cells illustrating the gate of neutrophils based on their high expression of Ly6G (Ly6G^{hi}) FIG. 6e shows pseudocolor plots of Annexin V and 7AAD staining in the neutrophil (Ly6G^{hi}) populations as defined by the gate in FIG. 6d in the blood samples of the saline- or HOAc-treated asthmatic mice. Numbers in the plots are percentages of the cell populations in their respective quadrants.

FIG. 7a shows line graphs illustrating the experimental autoimmune encephalomyelitis (EAE) clinical scores of individual C57BL/6 mice immunized with MOG35-55 peptide to induce EAE, followed by treatments with saline, or saline plus HCl, or HOAc for the period of 0-7 days after the initiation of the treatments.

FIG. 7b is a bar graph showing the average EAE clinical scores of the same mice in FIG. 7a on day 4 after the initiation of the treatments. Error bars are standard deviations. Statistical significance of the differences between saline- and acid-treated mice was determined by Student t test, ** (p<0.01).

FIGS. 8a-8d are microscopic views of H&E-stained spinal cords of representative mice of the different treatment groups described in FIG. 7a: saline (FIG. 8a, FIG. 8c), HOAc (FIG. 8b), HCl (FIG. 8d).

FIG. 9 is a line graph illustrating the percentages of mice with EAE. Prior to the onset of disease, the mice received i.p. (intraperitoneal injection) treatments with either saline or saline plus HOAc. The percentages of mice that developed EAE from 0 to 6 days after the initiation of treatments are shown.

FIG. 10a shows pseudocolor plots of the signals of Carboxyfluorescein succinimidyl ester (CFSE) and intracellular pH of lymph node cells cultured with IL-2 or IL-2 plus anti-CD3 antibodies.

FIG. 10b shows histograms of CFSE signals of the same cells in FIG. 10a.

FIG. 10c shows pseudocolor plots of the signals of CFSE and intracellular pH of lymph node cells cultured with IL-4 or IL4+LPS.

FIG. 10d shows histograms of CFSE signals of the same cells in FIG. 10c.

FIG. 11a is a schematic illustrating the schedules for OVA challenges and treatments with saline, or saline plus HCl, or HOAc of OVA-sensitized mice for the first set of experiments. Arrows indicate the time points for the challenges or treatments.

FIG. 11b is a bar graph illustrating the average total numbers of mediastinal lymph node (MLN) cells of mice of the different treatment groups depicted in FIG. 11a. Statistical significance between saline and other treatment groups was determined by Student t test, * (p<0.05), ** (p<0.01).

FIG. 11c is a schematic illustrating the schedules for OVA challenges and treatments with saline, or saline plus HCl, HOAc, or NaOH of OVA-sensitized mice for the second set of experiments. Arrows indicate the time points for the challenges or treatments.

FIG. 11d is a bar graph illustrating the average total numbers of MLN cells of mice of the different treatment groups depicted in FIG. 11c. Statistical significance between saline treatment and other treatment groups was determined by Student t test; * (p<0.05), ** (p<0.01).

FIG. 12a is a copy of FIG. 11a showing the schedules for challenges and treatments of the OVA-sensitized mice in the first set of experiments.

FIG. 12b is a set of pseudocolor plots illustrating two lymphocyte populations (Lym1 and Lym2) in the MLN cells of mice in the different treatment groups depicted in FIG. 12a. Numbers are the percentages of these populations.

FIG. 12c is a set of pseudocolor plots of the MLN cells of the mice in the different treatment groups, illustrating the CD4, CD8 T cells and B cells (CD19⁺), Q3, Q1, and Q5, respectively, in the Lym1 and Lym2 populations.

FIG. 12d shows histograms of Ki-67 expression by the Lym1, Lym2 populations and the B cells in the Lym1 and Lym2 populations of mice in the different treatment groups.

FIGS. 12e-l are a series of bar graphs based on FIG. 12d, illustrating the percentages of Ki-67^{hi} and Ki-67^{lo} cells in the different cell populations of mice of the different treatment groups.

FIG. 13a is a copy of FIG. 11c showing the schedules for OVA challenges and treatments of OVA-sensitized mice in the second set of experiments.

FIG. 13b is a set of pseudocolor plots illustrating Lym1 and Lym2 populations in the MLN cells of mice in the different treatment groups depicted in FIG. 13a. Numbers are the percentages of these populations.

FIG. 13c is a set of pseudocolor plots of the MLN cells of the mice in the different treatment groups, illustrating the CD4, CD8 T cells and B cells (CD19⁺), Q3, Q1, and Q5, respectively, in the Lym1 and Lym2 populations.

FIG. 13d shows histograms of Ki-67 expression by the Lym1, Lym2 populations and the B cells in the Lym1 and Lym2 populations of mice in the different treatment groups.

FIGS. 13e-l are a series of bar graphs based on FIG. 13d, illustrating percentages of Ki-67^{hi} and Ki-67^{lo} cells in the various cell populations.

FIG. 13m is a bar graph illustrating the mean fluorescence intensities of Ki-67^{hi} cells in the Lym2 populations and the B cells in Lym2 of mice in the different treatment groups.

FIG. 14a is a series of histograms of Ki-67 expression of live total lymphocytes, CD4, CD8 T cells and B cells (CD19⁺) of primary lymph node cells in vitro treated with saline or saline plus HCl, HOAc or NaOH in FBS.

FIG. 14b is a bar graph based on FIG. 14a, illustrating the percentages of Ki-67⁺ cells in the various different cell populations in the treatment groups.

FIGS. 15a and 15b are bar graphs illustrating the percentages of live cells of the human T and B cell leukemia cells Jurkat and Raji after in vitro treatments with saline or saline plus HCl, HOAc or NaOH in FBS.

FIG. 16 is a series of histograms illustrating the percentages of Ki-67⁺ Jurkat cells after treatments with saline, or saline plus HCl, HOAc or NaOH.

FIG. 17a is a series of histograms showing the expression Ki-67 in gated B lineage cells (CD19⁺) (upper panels) and non-B lineage cells (CD19⁻) (lower panels) in the non-granulocyte white bone marrow (WBM) cells of mice treated with i.p. injections of saline or saline plus HCl or HOAc. Numbers in the histograms are the percentages of the Ki-67 negative, low or high cells.

FIG. 17b is a series of histograms illustrating the expression of Ki-67 by T cells (CD3⁺) in the WBM cells of mice in the different treatment groups. Numbers in the histograms are the percentages of Ki-67 negative, low or high cells.

FIG. 17c is a series of histograms showing the expression of Ki-67 by erythroid cells (TER-119⁺) in the lymphoid and myeloid lineage negative (stained negative for B220, CD3, CD11b, CD11c, CD48, Ly6G) WBM cells of mice in the different treatment groups. Numbers in the histograms are the percentages of Ki-67 negative, low/medium or high cells.

FIG. 17d is a series of histograms showing the expression of Ki-67 by hematopoietic stem cells (HSC) (Sca-1⁺ c-kit⁺ CD150⁺) in the lineage negative (stained negative for B220, CD3, CD11b, CD11c, CD48, Ly6G and TER-119) WBM cells of mice in the different treatment groups. Numbers in the histograms are the percentages of the Ki-67 negative, low/medium or high cells.

FIG. 17e is a bar graph comparing the percentages of reduction of Ki-67^{hi} cells of the different cell lineages in the bone marrow by i.p. HOAc treatments.

FIGS. 18a-18d are a series of microscopic views of immunohistochemistry staining of Ki-67 and hematoxylin counter staining of lung tissue sections from asthmatic mice treated with saline, or saline plus HCl, HOAc, or NaOH.

FIGS. 19a-19d are microscopic views of immunohistochemistry staining of Ki-67 and hematoxylin counter staining of intestines of mice treated with i.p. injection of saline or saline plus HCl, HOAc, or NaOH.

FIG. 20a is a photograph of thymuses of asthmatic mice treated with saline, or saline plus HCl, HOAc, or NaOH.

FIG. 20b is a histogram showing Ki-67 expression in total thymocytes of an unimmunized mouse.

FIG. 21a is a bar graph showing the average total numbers of thymocytes of asthmatic mice treated with saline or saline plus NaOH or HOAc. Statistical significance of differences between saline and other treatment groups was determined by Student t test ** (p<0.01).

FIG. 21b is a set of pseudocolor plots illustrating the CD4⁻CD8⁻ double negative (DN), CD4⁺CD8⁺ double positive (DP), CD4 and CD8 single positive subpopulations of live thymocytes of representative mice of the different treatment groups.

FIG. 21c is a series of histograms showing the expression of Ki-67 in live total thymocytes and the subpopulations of thymocytes of mice in the different treatment groups.

FIGS. 21d and 21e are bar graphs based on data in FIG. 21c, illustrating the percentages of Ki-67^{hi} (FIG. 21d) and Ki-67^{lo} (FIG. 21e) cells in the live total thymocytes or the subpopulations of thymocytes.

FIG. 22a is a series of histograms showing the expression of Ki-67 in live total thymocytes and the subpopulations of thymocytes after in vitro treatments with saline, or saline plus NaOH, HOAc, or HCl in FBS.

FIG. 22b is a bar graph based on FIG. 22a, illustrating the percentages of Ki-67⁺ cells in live total thymocytes and subpopulations of thymocytes.

FIG. 23a is a bar graph showing the averages of total white bone marrow (WBM) cells from both tibias of mice treated with saline or saline plus NaOH.

FIG. 23b is a pair of pseudocolor plots illustrating the percentages of WBM cells in total bone marrow cells from representative mice in FIG. 23a.

FIG. 23c is a bar graph illustrating the average percentages of WBM cells in the total tibia bone marrow cells. Statistical significance between saline- and NaOH-treated mice was determined by Student t test, * (p<0.05).

FIG. 24a is a pair of pseudocolor plots of CD19 staining of non-granulocyte WBM cells of mice treated with saline or saline plus NaOH, illustrating B and non-B lineage cell populations. Bone marrows were harvested 1 day after the final treatments.

FIG. 24b is a bar graph based on FIG. 24a, showing the percentages of B and non-B lineage cells in the non-granulocyte WBM cells.

FIG. 24c is a pair of histograms of Ki-67 expression in the B lineage cells.

FIG. 24d is a bar graph based on FIG. 24c, showing the percentages of Ki-67 high, and low cells in the total B lineage cells.

FIG. 24e is a pair of histograms of Ki-67 expression in the non-B lineage cells.

FIG. 24f is a bar graph based on FIG. 24e, showing the percentages of Ki-67 high, and low cells in the total non-B lineage cells.

FIGS. 24g-j are histograms and bar graphs showing the expression of Ki-67 and the percentages of Ki-67⁺ cells in the B and non-B lineage cells of bone marrows harvested from mice 3 days after the final treatments with saline or saline plus NaOH.

FIG. 25a is a pair of pseudocolor plots showing the CD3⁺ T cell population in the WBM cells of mice treated with saline or saline plus NaOH.

FIG. 25b is a bar graph based on FIG. 25a, illustrating the percentages of the CD3⁺ T cells in the WBM cells.

FIG. 25c is a pair of histograms showing the expression of Ki-67 in the T cells.

FIG. 25d is a bar graph based on FIG. 25c, illustrating the percentages of Ki-67^{hi} and Ki-67^{lo} cells in the CD3⁺ T cells.

FIG. 26a is a pair of pseudocolor plots illustrating the CD11c⁺TCR⁻ cells in WBM cells of mice treated with saline or saline plus NaOH.

FIG. 26b is a bar graph based on FIG. 26a, illustrating the percentages of the CD11cTCR⁻ cells in the WBM cells.

FIG. 26c is a pair of histograms illustrating Ki-67 expression in the CD11c⁺TCR⁻ cells.

FIG. 26d is a bar graph based on FIG. 26c, illustrating the percentages of Ki-67⁺ cells in the CD11c⁺TCR⁻ cells.

FIG. 27a is a pair of pseudocolor plots showing Ly-6G⁺ cells in WBM cells of mice treated with saline or saline plus NaOH.

FIG. 27b is a bar graph based on FIG. 27a, illustrating the percentages of the Ly-6G⁺ cells in the WBM cells.

FIG. 27c is a pair of histograms showing the expression of Ki-67 in the Ly-6G⁺ cells.

FIG. 27d is a bar graph based on FIG. 27c, illustrating the percentages of Ki-67⁺ cells in the Ly-6G⁺ cells.

FIG. 28a is a pair of pseudocolor plots illustrating TER-119⁺ cells in lymphoid and myeloid negative (B220⁻ CD3⁻ CD11b⁻ CD11c⁻ CD48⁻ Ly⁻ 6G⁻) WBM cells of mice treated with saline or saline plus NaOH.

FIG. 28b is a bar graph based on FIG. 28a, illustrating the percentages of TER-119⁺ cells in the lymphoid and myeloid negative WBM cells FIG. 28c is a pair of histograms showing the expression Ki-67 in the TER-119⁺ cells.

FIG. 28d is a bar graph based on FIG. 28c, showing the percentages of Ki-67^{hi} and Ki-67^{lo} cells in the TER-119⁺ cells.

FIG. 29a is a pair of pseudocolor plots showing the Sca-1⁺c-kit⁺ stem cells in lineage negative (Lin⁻) (B220⁻ CD3⁻ CD11b⁻ CD11c⁻ CD48⁻ Ly-6G⁻ TER-119⁻) WBM cells of mice treated with saline or saline plus NaOH.

FIG. 29b is a bar graph based on FIG. 29a, illustrating the percentages of the Sca-1⁺c-kit⁺ stem cells in the Lin⁻ WBM cells.

FIG. 29c is a pair of pseudocolor plots illustrating the hematopoietic stem cells (HSC) (CD150⁺) and the multipotent progenitors (MPP) (CD150⁻) in the Sca-1⁺c-kit⁺ stem cells.

FIG. 29d is a bar graph based on FIG. 29c, illustrating the percentages of the HSC and MPP in the Sca-1⁺c-kit⁺ stem cells.

FIG. 29e is a pair of histograms showing the expression of Ki-67 in the HSC.

FIG. 29f is a bar graph based on FIG. 29e, illustrating the percentages of Ki-67^{hi} and Ki-67^{lo} cells in the HSC.

FIG. 29g is a pair of histograms showing the expression of Ki-67 in the MPP.

FIG. 29h is a bar graph based on FIG. 29e, illustrating the percentages of Ki-67^{hi} and Ki-67^{lo} cells in the MPP.

FIG. 30 is a bar graph comparing the folds of increase of the percentages of Ki-67^{+/hi} cells of the different cell lineages in the bone marrow by NaOH treatments.

FIG. 31a is a bar graph illustrating the average percentages of bodyweight reduction in mice after the treatments with saline, or saline plus HCl, HOAc, or NaOH.

FIG. 31b is a set of photographs of the fat pads of representative mice in the different treatment groups.

FIG. 31c is a bar graph illustrating the average fat/bodyweight ratios of the mice in the different treatment groups. Error bars are standard deviations. Statistical significance of differences between saline and other groups was determined by Student t test, ** (p<0.01).

FIG. 32a is a set of photographs of the entire intestines of representative mice treated with saline, or saline plus HCl, HOAc, or NaOH.

FIG. 32b is a bar graph showing the average lengths of the intestines of mice of the different treatment groups. Statistical significance of differences between saline and other treatment groups was determined by Student t test, ** (p<0.01).

FIG. 32c is a bar graph illustrating the average percentages of reduction of the intestinal length of the acid- or alkaline-treated mice as compared with the saline-treated mice.

FIGS. 33a and 33b are bar graphs illustrating the average weights of the livers and kidneys, respectively, of the mice in the different treatment groups. Error bars are standard deviations.

FIG. 34 is a set of photographs showing the improvements of 2 lesions of poison ivy-induced allergic contact dermatitis on the left forearm of a human subject after 4 topical treatments with HOAc per day for 2 consecutive days (day 0 and day 1).

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter, for example, use of different pH modifiers or cell proliferation inhibitors, their doses, concentrations in a composition and dosing regimens than those described in the examples of the embodiments.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

### Definitions

Following long-standing patent law convention, the terms “a”, “an”, and “the” refer to “one or more” when used in the subject specification, including the claims. Thus, for example, reference to “a cell” can include a plurality of such cell, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used in this invention, the term “about”, when referring to a value or to an amount of mass, weight, time, volume, concentration, dose, and/or percentage can encompass variations of, in some embodiments +/−0.01-50% from the specified amount, as such variations are appropriate in the disclosed packages and methods.

“Acute Respiratory Distress Syndrome (ARDS)” as used in this invention refers to acute inflammation in the lungs, leading to fluid build-up in the lungs and impairment of the respiratory function of the lungs. The various causes of ARDS include (but are not limited to) sepsis, bacterial or viral pneumonia, pancreatitis, fume inhalation, trauma, and embolism.

The terms “administration,” “administer,” or “administering” as used in this invention refer to the delivery of a composition comprising pH modifier(s) and/or cell proliferation inhibitor(s) into or on the body of a subject, either systemically or locally to a specific anatomical location via any of a variety of routes and means of administration. Suitable routes and means of administration include (but are not limited to) intravenous (i.v.) injection/perfusion, intra-peritoneal (i.p.) injection, intratracheal (i.t.) injection, instillation or spray, inhalation of aerosols or vapors, topical application, intranasal spray, intradermal injection, subcutaneous injection, and rectal application. The means of administration also include the velocity of administration, i.e., the amount of pH modifier(s) and/or cell proliferation inhibitor(s) delivered to a subject in a unit of time. For example, a dose of 420 μmoles HOAc/kg body weight in a composition of 50 mM HOAc in saline may be administered to a subject of 75 kg body weight by intravenous infusion at a velocity of 0.525 mmoles (or 10.5 ml of the composition) per minute. Administration to a specific anatomical location may be carried out with or without assistance with guiding/navigation technologies such as imaging technologies. The selection of pharmaceutically acceptable routes and means of administration depends on the nature of the disease, the tissue(s) and/or organs affected by the disease, and should be determined by a subject's attending health care provider(s) within the scope of sound medical judgment.

“Adoptive cell transfer” refers to the transplantation of cells into a subject. The cells are most commonly but not exclusively derived from the blood or bone marrow. For example, in autologous cancer immunotherapy, T cells are extracted from the blood of the subject, genetically modified and cultured in vitro and returned to the same subject. Comparatively, allogeneic transfer involves cells from a separate donor, who except for an individual developed from the same fertilized ovum as the recipient (e.g., an identical twin), is genetically different from the recipient. The cells, when transferred, may be in their original tissues such as blood or bone marrow or have been isolated; they may or may not have been cultured or modified in vitro.

“Allergic disease” as used in this invention refers to diseases caused by inflammatory and/or immune responses to allergen exposure. The immune responses to the allergen(s) are typically but not exclusively characterized by the production of the type 2 cytokines, e.g., IL-4, 5 and 13, and IgE antibodies. Allergic diseases include (but are not limited to) allergic rhinitis, allergic asthma, allergic dermatitis, food allergy and urticaria.

The term “anatomical location” refers to any spatial point inside or on the body of a subject. For example, an anatomical location can be a location in the interstitial space of a tissue, the lumen of an organ, a body cavity, the space inside the blood vessels, an area of the mucosa, or skin.

The term “aqueous environment” refers to extracellular and/or intracellular fluid at any anatomical location within or on a subject, intracellular fluid of in vitro cultured cells, cell culture medium, and any aqueous solutions prepared in vitro. Examples of extracellular fluids include (but are not limited to) interstitial fluid, plasma, lymphatic fluid, mucus, bronchoalveolar fluid, cerebrospinal fluid, synovial fluid, fluids of chest and abdominal cavities.

The terms “as example(s)”, “for example(s)” and “such as” as used in this invention, are not limiting, and do not exclude other subject matters similar to the example(s).

The term “Atherosclerotic Cardiovascular Diseases” or “ASCVD” as used in this invention refers to diseases caused by the formation of atheromatous plaques underneath the endothelium of the arteries. Inflammatory and/or immune responses play crucial role in the formation of an atheromatous plaque, in which monocytes and smooth muscle cells migrate to the sub-endothelial space of the blood vessels. The monocytes further develop into macrophages. Both macrophages and smooth muscle cells proliferate and ingest lipids, the latter of which is the main cause of lipid deposition in the atheromatous plaque. The eventual consequence of atheromatous plaque formation is the limitation or blockade of blood flow in the arteries. Depending on which artery or arteries are affected, different organs or tissues may be damaged, leading to different ASCVD, including (but not limited to) coronary heart disease, stroke, peripheral arterial disease, and chronic kidney disease.

“Autoimmune disease” as used in this invention refers to diseases that are characterized by immune and inflammatory responses to one or more components of a subject's own tissues, cells, and/or bodily fluids. Examples of autoimmune diseases include (but are not limited to) multiple sclerosis, Anti-NMDA (N-methyl-D-aspartate) receptor encephalitis, autoimmune vasculitis, Giant cell myocarditis, Grave's disease, lupus, inflammatory bowel diseases, rheumatoid arthritis, psoriasis, Hashimoto's thyroiditis.

“Cell proliferation inhibitor” as used in this invention refers to any molecule or drug other than a pH modifier that inhibits intestinal epithelial cell renewal. They can be classified in two categories, the general inhibitors and inhibitors of the signaling pathways operating in the crypt of the intestinal villus. The former includes (but is not limited to) inhibitors and antagonists of DNA synthesis, cyclin-dependent kinases (CDKs) and Myc. For examples, some inhibitors and antagonists in this category include (but are not limited to) the DNA synthesis inhibitors 5-fluorouracil, gemcitabine, thiarabine; the CDK inhibitors palbociclib, ribociclib, abemaciclib; the Myc inhibitors OmoMYC, APTO-253, GQC-05, SYUIO-05, DC-34; and others that are described at https://www.selleckchem.com and in references (33-38). The latter category includes (but is not limited to) inhibitors and/or antagonists of the Wnt-β-catenin signaling pathway, the EGFR signaling pathway and the Notch signaling pathway. For examples, the inhibitors and antagonists in this category include (but are not limited to) the Wnt signaling inhibitors LGK974, vantictumab (OMP-18R5), PRI-724; the EGFR signaling inhibitors AG1478, PD98059, cetuximab; the Notch signaling inhibitors MK-0752, OMP-52M51 and OMP-21M18; and others that are described at https://www.selleckchem.com and in references (39-45). These inhibitors and/or antagonists have not been used for therapy of overweight and/or obesity, and are repurposed for such application in this invention.

“Composition” as used in this invention refers to a pharmaceutical formulation comprising certain amounts of one or more pH modifiers and/or cell proliferation inhibitors together with or without one or more other medicines formulated with one or more pharmaceutically acceptable solvents, solutions, excipients, and/or carriers. When more than one pH modifiers and/or cell proliferation inhibitors are used in a composition, the amount and/or concentration of each of them may be the same or different. The concentration of a pH modifier or cell proliferation inhibitor in a composition may range from 0.001 μM to 10M (e.g. 0.01, 1, 10, 25, 50, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500 mM).

While maintaining the therapeutic or preventive efficacy of a composition, the concentration(s) of pH modifier(s) and/or cell proliferation inhibitor(s) must be set at such levels that the composition meets the standard of “pharmaceutically acceptable”. As further defined later, the amount of a composition administrated to a subject at a given time comprises one dose of the pH modifier(s) and/or cell proliferation inhibitor(s). A composition may be in any of a variety of physical forms, including (but not limited to) liquid, aerosol, vapor, cream, gel, capsule, tablet, powder, granules, and the like. All or some of the components of a composition may be pre-mixed or supplied separately and mixed before use. It will be understood that the composition to be used on a given subject at a given time will be decided by the subject's attending health care provider(s) within the scope of sound medical judgment. As examples, in some embodiments, the composition is a solution of about 175 mM HOAc or HCl in saline; in some embodiments, the composition is a solution of about 87.5 mM HOAc (acetic acid), HCl (hydrochloric acid) or NaOH (sodium hydroxide) in saline; in some embodiments, the composition is a solution of 5.25M HOAc in water.

The term “deplete”, “depleting”, or “depletion”, as used in this invention refers to the reduction and/or elimination of certain cell population(s) systemically and/or at a specific anatomical location.

The term “disease” as used in this invention refers to an abnormal condition that impairs the structure and/or function of one or more tissues and/or organs of a subject. In some embodiments, the disease can be caused by either external factors such as microbial agent(s) or other harmful substance(s), e.g., allergen(s) and cigarette smoking; or internal factors such as self-antigens. For examples, diseases relevant to the presently disclosed subject matter include (but are not limited to) asthma, pneumonia, chronic obstructive pulmonary disease (COPD), acute respiratory distress syndrome (ARDS) of various causes (e.g., sepsis, bacterial or viral pneumonia, fume inhalation, pancreatitis, embolism), muco-obstructive lung diseases, atherosclerotic cardiovascular diseases (ASCVD), allergic diseases (e.g., allergic asthma, allergic rhinitis, allergic dermatitis, food allergies), autoimmune diseases (e.g., multiple sclerosis, Anti-NMDA (N-methyl-D-aspartate) receptor encephalitis, autoimmune vasculitis, Giant cell myocarditis, Grave's disease, lupus, inflammatory bowel diseases, rheumatoid arthritis), vasculitis, infections at any anatomical location (such as but not limited to the blood, lungs, and/or airways), malignant or benign neoplastic diseases, anemia, loss of blood due to trauma or surgery, and obesity. The term “disease” is used interchangeably with “disorder” or “clinical condition”.

A “dose” or “dosage” as used in this invention refers to the amounts of one or more pH modifiers and/or cell proliferation inhibitors in a composition administered to a subject at one time. When more than one pH modifiers and/or cell proliferation inhibitors are used, the amount of each of them in a dose may be the same or different (e.g., a dose may comprise 200 μmoles/kg body weight of HOAc and 100 μmoles/kg body weight of HCl). For a given subject, the dose is predetermined by the attending health care provider(s) within the scope of sound medical judgment. The amount of a single pH modifier or cell proliferation inhibitor in a dose may range from about 0.001 nmoles/kg body weight to about 500 mmoles/kg body weight (e.g., about 0.01, 1, 10, 25, 50, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 μmoles/kg body weight). For examples, in some embodiments the dose is about 420 μmoles/kg body weight of HOAc or HCl; in some embodiments, the dose is about 700 μmoles/kg body weight of HOAc, HCl or NaOH. In some embodiments, the determination of the dose may be assisted by assays that compare the viabilities and/or the percentages of inflammatory and/or proliferating cells in peripheral blood cells after treatments with and without the pH modifiers and/or cell proliferation inhibitors; a dose is tentatively determined as effective when the comparison shows significant difference.

The terms “dosing” and “dosing regimen” as used in this invention are interchangeable, and refer to the administration to a subject, over a period of time, of a set of one or more doses of pH modifier(s) and/or cell proliferation inhibitor(s) that show significant probability of achieving therapeutic and/or preventive effect. The administration of individual doses is separated by time intervals between two sequential doses. The lengths of the time intervals between any two sequential doses may be the same or different and may range from about 1 second to about 1 year. The dose administered at one time may be the same or different from the dose administered at another time. It will be understood that the dosing regimen for a given subject is determined by the attending health care provider(s) within the scope of sound medical judgment. For example, in some embodiments, the dosing is intratracheal instillation of a dose of about 420 μmoles/kg body weight of HOAc or HCl in a composition of 175 mM HOAc or HCl in saline every other day for a total of 3 doses; in some embodiments, the dosing is intra-peritoneal injection of a dose of about 700 μmoles/kg body weight of HOAc, HCl, or NaOH in a composition of about 87.5 mM HOAc, HCl, or NaOH in saline every other day for a total of 3 doses.

The term “drug addiction” refers to a habitual psychological physiological dependence, or both on a substance or a mixture of different substances that is beyond voluntary control, where the substance includes but as not limited to, alcohol, amphetamine, cocaine, heroin, inhalants, morphine, nicotine, opiates.

The terms “effector immune cells” and “effector inflammatory cells” are interchangeable, and refer to immune cells that mediate inflammatory and/or immune responses. For examples, the effector immune cells include (but are not limited to) the functional subsets of immune cells that mediate inflammatory and/or immune responses such as the different T helper subsets (e.g., Th1, Th2, Th17), cytotoxic CD8 T cells, B cells producing antigen specific antibodies, innate lymphoid cell subsets (e.g. ILC1, ILC2, ILC3) and phagocytes. However, effector immune cells also include immune cells that could not be characterized as any known functional subset. Depending on the diseases, effector immune cells can be either pathological cells or protective normal cells. For example, allergen specific Th2 cells are pathological cells in allergic diseases, whereas worm specific Th2 cells are protective normal cells in parasitic infectious diseases.

The term “enzyme” as used in this invention refers to molecules or molecular aggregates that are responsible for catalyzing chemical and/or biological reactions. Such molecules are typically proteins, but can also comprise short peptides, RNAs, and other molecules.

“Fat mass” as used in this invention refers to the weight of adipose tissues in the entire body or certain areas of the body of a subject, for example, the weight of all the fat pads collected from a mouse subject.

The terms “functional subset of immune cells”, “functional subset of inflammatory cells”, “immune cell functional subset” and “inflammatory cell functional subset” are interchangeable as used in this invention and refer to any currently known or unknown subpopulation of innate or adaptive immune cells that produce a unique set of one or more secreted molecules, e.g., cytokines, mediators and/or antibodies, and/or express a unique set of one or more cell surface molecules, which mediate unique immunological and/or inflammatory functions. Functional subsets of immune cells include but are not limited to the following examples: T helper cell subsets (e.g., Th1, Th2, Th17) (46), cytotoxic CD8 T cells, thymus-derived regulatory T (nTreg) (47), and inducible (derived from peripheral naïve CD4 T cells) regulatory T (iTreg) cells (48), CD1d^{high}IL-10⁺ regulatory B (Breg) cells and IL-6⁺ effector B (Beff) cells (49); B cells producing different classes or subclasses of antibodies (e.g., IgE-producing B cells, IgG2a-producing B cells); the different subsets of innate lymphoid cells (e.g., ILC1, ILC2 and ILC3 subsets) (50); M1 and M2 macrophages (51), etc. While the T helper cells and effector B cells promote inflammatory and/or immune responses, the nTreg, iTreg and Breg cells suppress inflammatory and/or immune responses. Antibodies produced by B cells can also determine the role of B cells in a disease. For example, IgE-producing B cells are pathological cells in allergic diseases, because IgE activates mast cells to produce histamines. On the other hand, M1 macrophages are pro-inflammatory and/or anti-tumor, whereas M2 macrophages promote tumorigenesis and wound healing. (51). Different functional subsets of immune and/or inflammatory cells can be pathological cells or normal cells in various diseases.

The terms “functional subset of immune cells with anti-inflammatory, immune suppressive activities” and “functional subset of inflammatory cells with anti-inflammatory, immune suppressive activities, or both” are interchangeable as used in this invention and refer to one of a subcategory of functional subsets of immune cells that suppresses inflammatory response, immune response, or both inflammatory and immune responses. They include but are not limited to regulatory T and B cells, type 1 regulatory T (Tr1) cells, (52), myeloid-derived suppressor cells, (53), and the like.

“Hematopoietic cells” as used in this invention refer to cells originated from the blood, lymphoid tissues, and bone marrow. Hematopoietic cells include immune cells, white bone marrow cells, various leukocytes and erythroid cells.

The term “host target cell” as used in this invention refers to a cell infected by microbes (e.g., viruses, bacteria and protozoa) in which the microbes replicate and/or produce substances harmful to a subject. A host target cell is a pathological cell in an infectious disease. A cell of the same cell type but not infected by the microbes is not a host target cell, therefore is a normal cell.

“Hyperplastic, hyperactive structural functional cells” refer to cells of a tissue or organ that play a structural role, a functional role, or both structural functional role of the tissue or organ; and are undergoing dysregulated proliferation, perform their function excessively, or both proliferate and perform function excessively. Examples of such cells include but are not limited to hyperplastic type 2 pneumocytes in allergic asthma, epithelial cells in the airways that hyper-produce mucus, and hyperplastic thyrocytes that produce excessive thyroid hormone in Grave's disease.

“Immune response” as used in this invention refers to the activation of innate, adaptive, or both innate and adaptive immune cells in specialized lymphoid organs/tissues such as the lymph nodes, spleen and peyer's patches, and the anatomical location of antigen exposure; and the cellular and molecular consequences thereof. Innate immune cells are activated by receptors other than antigen receptor. The innate immune cell receptors may recognize and be activated by a variety of ligands, e.g., microbial products such as pathogen-associated molecular patterns (PAMPs), endogenous danger-associated molecular patterns (DAMPs), or antibody-antigen complexes. The consequences of innate immune responses include (but are not limited to) the release of inflammatory mediators, e.g., cytokines, prostaglandins, and histamines; and up-regulation of certain immunologically important surface molecules, e.g., the costimulatory molecules, major histocompatibility complex (MHC) antigens or human leukocyte antigens (HLA). The activation of the adaptive immune cells (mainly B and T lymphocytes and NKT cells) primarily results from the engagement of antigen receptors (BCR or TCR) with antigens or superantigens but may also be caused by stimulation by B or T cell mitogens (e.g., Lipopolysaccharides, Concanavalin A), and also includes by-stander activation by cytokines. The cellular and molecular consequences of the activation of adaptive immune cells include, (but are not limited to), clonal expansion of antigen specific lymphocytes, expression of effector cell surface molecules, antibodies, cytokines and/or other mediators. The secreted and cell surface molecules expressed by the immune cells upon activation mediate the immune cells' immunological and/or inflammatory functions, and/or act on other cells in the local microenvironment and/or systemically. In the case of infection, the immune response as defined herein is crucial for the clearance of the infection in a subject but may also cause tissue damage when it is said to be overzealous.

The term “infectious disease” refers to a disease that can be transmitted from one subject to another, and is caused by a microbial agent (e.g., pneumonia). The microbial agents can be bacteria, viruses, fungi or parasites. Not only the microbial agents themselves can directly harm the infected subject, overzealous inflammatory and/or immune responses to the microbial agents can also cause pathology and pathophysiology, whereas adequate inflammatory and/or immune responses to the microbes are necessary for the clearance of the infection.

The terms “inflammatory cells” and “immune cells” as used in this invention are interchangeable and refer to both the innate and adaptive immune cells and include cells of the lymphoid and myeloid lineages. Inflammatory cells may circulate through the blood and lymphatic systems and may migrate to and take residence in specific tissues or anatomical locations. During and after the migration to an anatomical location, inflammatory cells may undergo structural and/or functional changes, which do not alter their identities as inflammatory cells as defined herein. Adaptive immune cells are cells that express antigen receptors, i.e., either the B cell receptor (BCR) or T cell receptor (TCR) that recognize their cognate antigens; they include (but are not limited to) B and T lymphocytes and NKT cells. Innate immune cells are immune cells that do not express BCR or TCR; they include (but are not limited to) neutrophils, basophils, eosinophils, monocytes, macrophages, mast cells, dendritic cells, Langerhans cells, natural killer cells, and innate lymphoid cells.

“Inflammatory disease” as used in this invention refers to a disease or condition, in which inflammatory and/or immune responses or the lack or insufficiency thereof play a pathogenic role.

“Inflammatory response” and “inflammation” are interchangeable as used in this invention and refer to the increase and/or activation of inflammatory cells in anatomical location(s) other than the specialized lymphoid organs/tissues as a result of exposure to stimulants, and the cellular and molecular consequences thereof. Stimulants include (but are not limited to) microbes, allergens, self-antigens, injured tissues, chemical irritants, etc. In some clinical conditions, (e.g., some autoimmune and allergic diseases) the exact nature of the stimulants may be unknown. Inflammatory response comprises both the innate and adaptive immune responses outside the specialized lymphoid organs/tissues. The cellular and molecular consequences of inflammation include (but are not limited to) the activation of inflammatory cells, release of inflammatory mediators and/or cytokines, expression of effector cell surface molecules, interactions among inflammatory cells, interactions between inflammatory cells and other cells in the microenvironment, effects of the inflammatory mediators and/or cytokines and effector cell surface molecules on tissue cells locally and systemically (e.g., tissue cell hyperplasia, mucus hyper-secretion, organ failure).

The term “inhibitor” refers to a molecule or a mixture of different molecules that exerts the effect of alteration, interference, reduction, down-regulation, blocking, suppression, abrogation, or degradation (directly or indirectly) of the expression, amount, or activity of an enzyme, membrane transporter or ion channel responsible for the change and/or maintenance of pH in and/or around a cell.

The “intestinal villi” are small, finger-like projections that extend into the lumen of the intestines. Each villus has many microvilli projecting from the enterocytes of its epithelium. The villi and microvilli increase the internal surface area of the intestinal walls, making available a greater surface area for absorption of nutrients.

“Membrane transporter” as used in this invention refers to a biomembrane-associated structure that is capable of moving ions and/or molecules across a lipid membrane of a cell.

“Minimally” or “minimum” are used in this invention to describe the therapeutic or preventive regimens using pH modifier(s) or cell proliferation inhibitor(s) that have a low inhibitory or negative effect on normal cells while achieving a desirably strong inhibitory or negative effect on pathological cells; or conversely have a desirably strong stimulatory or positive effect on normal cells while having low stimulatory or positive effect on pathological cells of a disease. In some embodiments, “minimally” (e.g., minimally increased) can refer to an amount of the effect on pathological cells at about 0.001-70 percent (e.g., at about 0.001, 0.005, 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, or 10 percent) of that on normal cells, or vice versa.

“Muco-obstructive lung disease” refers to any disease that mucus secretion obstructs the airways of a subject, including (but not limited to) chronic obstructive pulmonary disease (COPD), cystic fibrosis, primary ciliary dyskinesia, and non-cystic fibrosis bronchiectasis.

“Neoplasia” or “neoplastic” are used in this invention to describe dysregulated, benign or malignant cell proliferation. Solid and nonsolid tumors are neoplastic diseases. Malignant solid and nonsolid tumors are synonymous with cancers.

The term “normal cell” as used in this invention refers to a cell that does not play a pathogenic or adverse role in a disease. A normal cell may be a cell that plays a protective or preventive role against a disease, or a cell irrelevant to the disease. The functional subsets of immune cells can be normal cells or pathological cells depending on their roles in a particular disease. For example, an allergen-reactive Th2 cell is a pathological cell in an allergic disease, but a parasite-reactive Th2 cell is a protective normal cell in a disease of helminth infection.

The term “obesity” or “obese” as used in this invention refers the physical condition of a subject that fits the contemporary definition of obesity by health authorities such as the World Health Organization, which currently defines obesity as “abnormal or excessive fat accumulation that presents a risk to health. A body mass index (BMI) over is considered overweight, and over 30 is obese” (https://www.who.int/health-topics/obesity). “Body mass index” is the ratio of body weight (in kg) to the square of height (in meters). In the mouse model, obesity refers to the excessive fat accumulation as a result of feeding on high fat diet.

“Overzealous” is used in this invention to characterize inflammatory and/or immune responses that impair the structure and/or function of the tissue(s) and/or organ(s) of a subject. Immune and/or inflammatory responses to allergens and self-antigens in allergic and autoimmune diseases, respectively, are generally considered as overzealous immune and/or inflammatory responses. Immune and/or inflammatory responses to infection can also be overzealous when such responses damage normal tissue or organs.

The term “pathogen” is used interchangeably with infectious agents, and refers to an organism that can be transmitted from one subject to another and causes infectious disease, for example, any of pathogenic viruses, bacteria, fungi and parasites.

The term “pathogenesis” as used in this invention refers to the processes of disease initiation, progression, exacerbation, and relapse, and/or the underlying mechanisms.

“Pathological cell” as used in this invention refers to a cell that contributes to the pathogenesis of a disease. For examples, pathological cells may include (but are not limited to) inflammatory cells, hyperplastic, hyperactive structural functional cells of tissue or organ, and host target cells. During the clinical course of an infectious disease, an immune cell directly or indirectly responding to the infectious agents is a pathological cell when the primary concern is overzealous immune and/or inflammatory response, whereas it is a protective normal cell when the primary concern is insufficient protective immunity, or when the immune and/or inflammatory responses are adequate for the clearance of infection without causing collateral damage to tissues. Depending on what functional subset it belongs to, an immune cell can be either a pathological or normal cell. For examples, a T helper type 17 (Th17) cell reactive to self-antigen is a pathological cell in certain autoimmune diseases such as multiple sclerosis; and a T helper type 2 (Th2) cell reactive to allergen is a pathological cell in allergic asthma; whereas a natural regulatory T (nTreg) or inducible regulatory T (iTreg) cell is a protective normal cell against autoimmune or allergic disease. IgE- but not IgG-producing B cell is a pathological cell that produces antibodies to activate mast cells to release histamine in allergic diseases. M1 and M2 macrophages play opposite roles of anti- and pro-tumorigenesis, and thus are normal cells and pathological cells in neoplastic diseases, respectively.

“pH modifier” as used in this invention refers to any pharmaceutically acceptable compound that can alter, or help resist the change of, the pH of an aqueous environment. Suitable pH modifiers include (but are not limited to) inorganic and organic acids, inorganic and organic bases. For examples, in some embodiments, the pH modifiers are HCl, HOAc or NaOH. The pH modifiers can also be inhibitors or stimulators of the functions and/or expression of enzymes, membrane transporters and ion channels responsible for facilitating the synthesis and/or cross-membrane movement of certain contents of the aqueous environments (e.g., proton, HCO₃⁻, monocarboxylates, etc.), which contribute to the change or maintenance of the pH of the aqueous environments in and/or around a cell. For examples, such inhibitors and/or stimulators include (but are not limited to) the Na⁺/H⁺ exchanger inhibitors amiloride and derivatives, and cariporide; the vacuolar H⁺-ATPase inhibitors lansoprazole, plecomacrolide bafilomycin-A1, concanamycin-A, benzolactone enamides, and archazolid; the carbonic anhydrase inhibitors sulfonamides, coumarin and derivatives, psoralen and derivatives, and 1,2,4-oxadiazoles derivatives; the carbonic anhydrase stimulators L-/D-phenylalanine, carnosine and derivatives; the Na⁺/HCO₃⁻ cotransporter inhibitor S0859; the monocarboxylate transporter inhibitors phoretin, quercetin, lonidamine, α-cyano-4-hydroxycinnamate, L-lactate, γ-hydroxybutyrate; and others described at https://www.selleckchem.com and in references (54-63). The said inhibitors and stimulators are repurposed as pH modifiers for applications to the diseases or conditions concerned in this invention. The repurpose is based on the novel findings disclosed in this invention: the depletion of pathological cells but preservation of normal structural functional cells of the tissues by pH modifiers; depletion of proliferating nonmalignant and malignant, pathological cells by pH modifiers (e.g. HOAc) but increase of the population and/or proliferation of normal cells (inflammatory and/or non-inflammatory) by other pH modifiers (e.g. NaOH); a positive correlation between high pH and high rates of cell proliferation, and correlation between low pH or acid environment and apoptosis. Thus, the inhibitors and stimulators are repurposed to selectively deplete and suppress the population and/or proliferation of nonmalignant and/or malignant pathological cells, or conversely to selectively increase the population and/or proliferation of protective normal cells.

The pH modifiers further include pH buffering agents, for examples, histidyl dipeptide, TAPS ([tris(hydroxymethyl)methylamino]propanesulfonic acid), amino, Tris (tris(hydroxymethyl)aminomethane), PIPES (piperazine-N,N′-bis(2-ethanesulfonic acid)), MES (2-(N-morpholino)ethanesulfonic acid), and the like.

The term “pharmaceutically acceptable” refers to the characteristics of any molecule and/or compound or a mixture of different molecules or compounds that, within the scope of sound medical judgment, is suitable for use in a subject without causing excessive toxicity, allergic response, irritation, and/or other problem or complication, commensurate with reasonable risk/benefit ratio.

“Precursor” as used in this invention refers to any cell that exists at a time prior to another cell in the process of the development or maturation of a cell or cell lineage. A precursor includes but is not limited to a stem cell or progenitor commonly referred to in scientific literature.

“Prevention,” “prevent,” “preventing,” and “preventative” as used in this invention describe any medical intervention that applies to a subject susceptible to a disease but not experiencing symptoms of the disease for the purpose of decreasing or eliminating the likelihood of the development or relapse of the disease. In some embodiments, the terms also refer to the delaying of the onset or relapse of a disease.

“Proliferation” as used in this invention refers to the increase of the number of cells by cell division and the survival of the dividing cells.

The term “pulmonary inflammation” as used in this invention refers to inflammation prominently manifested in the lungs and/or airways. However, the inflammation can also concurrently occur outside the lungs and/or airways, e.g., in blood and anatomical locations of primary infection, and accompanied by immune responses in lymphoid tissues and organs. Diseases associated with pulmonary inflammation include (but are not limited to) asthma, pneumonia, chronic obstructive pulmonary disease, muco-obstructive lung diseases, acute respiratory distress syndromes of various causes.

The terms “selectively” and “selective” as used in this invention describe pH modifiers and/or cell proliferation inhibitors that work exclusively or have stronger effect on pathological cells than normal cells, or vice versa.

The data disclosed in this invention demonstrated that different cells respond differently to the same pH modifier; and the cells' responses to the same pH modifier are also determined by the doses or concentration of the pH modifier and the duration of treatment with the pH modifiers. These observations provide the guide for achieving the selectivity of the effects of pH modifiers on pathological or normal cells. Thus, the selectivity of the effect of pH modifiers is achieved by choosing pH modifiers and routes and means of administration; titrating their doses and dosing regimens; and titrating the concentrations of the pH modifier(s) and/or cell proliferation inhibitor(s) in a composition. The choice of pH modifiers can be further guided by the differential expression and/or activities between pathological and normal cells of enzymes, membrane transporters, or ion channels responsible for the change or maintenance of pH in and/or around the cells. For a given subject, these selective decisions are made by the subject's attending health care provider(s) within the scope of sound medical judgment, adhering to the guidelines or principles set forth herein. Similarly, cell proliferation inhibitor(s), their doses and dosing regimens are chosen to achieve the goal of selectively suppressing intestinal epithelial renewal while avoiding or minimizing their effects on cell proliferation in other tissues or organs. However, under some special circumstances where the overall risk/benefit ratio for a given subject may warrant the use of the pH modifiers or cell proliferation inhibitors even if their effects on pathological and normal cells are equal or opposite to what are described above.

The term “stimulator” as used in this invention refers to a molecule or a mixture of different molecules that increases the expression or function/activity of one or more enzymes, membrane transporters, and/or ion channels responsible for the change and/or maintenance of pH in and/or around a cell.

The term “subject” as used in this invention refers to a human being or non-human animal (e.g., mouse, dog, cat, cattle, horse). A human subject can be a healthy person or a patient seeking diagnosis, treatment, and/or prevention of a disease. When a subject is a human being, the term “subject” is used interchangeably with “individual”, “person”, or “patient”.

“Suffering from” when used in the context of a disease (e.g., suffering from a disease) refer to the experience of one or more symptoms of the disease by a subject.

The term “suppress”, “suppressing” or “suppression” as used in this invention refers to inhibiting, interfering with, slowing down, decreasing, or preventing a process (e.g. cell proliferation), an action, a reaction, a function, a cell population, or combinations thereof.

The term “susceptible to” as used in this invention refers to the presence of any number of risk factors for a disease in a subject when the subject is asymptomatic or in remission of the disease. Risk factors for a disease are factors that the medical community considers increase the chance for a subject to develop the disease or to relapse. Risk factors include (but are not limited to) age, genetic background, family history, environmental factors, lifestyle, and/or physical condition of a subject. A subject may be susceptible to a disease even if the subject has never been diagnosed with or has recovered from the disease. In the context of an infectious disease, all subjects are considered to be susceptible to the disease unless there is medical evidence of protective immunity or natural resistance (e.g., CCR5 null mutation for HIV AIDS) to the disease in a given subject.

The term “titrate” or “titrating” refers to the adjusting of the dose(s), dosing regimen(s) or the concentration(s) in a composition, of pH modifier(s) or cell proliferation inhibitor(s) to achieve selective therapeutic or preventive effect. The titration may be performed manually by health care provider(s) in response to the results of one or more tests and/or the observation of a subject's response to the pH modifier(s) or cell proliferation inhibitor(s); or may be performed automatically under the control of a computer programmed to perform the test(s) or the observation and adjust the dose(s), dosing regimen(s), or concentration(s) according to the result(s) of the test(s) and/or observation(s).

The term “vaccination” as used in this invention refers to the introduction of one or more vaccines into the body of a subject for the purpose of prevention or treatment of a disease.

The term “vaccine” refers to a therapeutic or prophylactic pharmaceutical formulation that includes one or more components against which a vaccinated subject is induced to mount immune responses, preferably protective immune responses. For example, such a component could be an antigenic component of a pathogen or a cancer cell, or a nucleic acid encoding such an antigenic component.

In addition to the terms defined in the preceding paragraphs, additional terms are defined throughout this invention. The Definitions also serve as guidelines and principles for the selection of pH modifier(s) and/or cell proliferation inhibitor(s), their dose(s), dosing regimen(s), and concentration(s) in a composition. Each paragraph, section, or definition of term in the Detailed Description applies to any other paragraph, section and definition of term of this invention.

### Treatments and/or Prevention of Pulmonary Inflammation and/or Allergic Diseases

In some embodiments, a composition comprising one or more pH modifiers is administered to a subject suffering from or susceptible to diseases of pulmonary inflammation. While not bound by any particular theory, the pH modifiers selectively deplete or suppress the population and/or proliferation of the pathological cells of the diseases to dampen overzealous inflammatory and/or immune responses within and outside the lungs and airways. Overzealous pulmonary inflammation and/or immune responses are common causes of many pulmonary diseases resulting from exposure to infectious agents, allergens, and harmful substances (e.g., cigarette smoke). For example, such pulmonary diseases include (but are not limited to) asthma, pneumonia, chronic obstructive pulmonary disease (COPD), muco-obstructive lung diseases, and acute respiratory distress syndrome (ARDS) of various causes (e.g., sepsis, bacterial or viral pneumonia, fume inhalation, pancreatitis, embolism). The overzealous pulmonary inflammation is mediated by effector inflammatory cells, which are the major pathological cells in the diseases of pulmonary inflammation.

In some embodiments, one or more pH modifiers can be administered to a subject suffering from an allergic disease with pulmonary inflammation. For examples, in some embodiments, a dose of about 420 μmoles/kg body weight of acetic acid (HOAc) or hydrochloric acid (HCl) in a composition of 175 mM HOAc or HCl in saline is administered via intratracheal instillation to an asthmatic subject every other day for a total of 3 doses; in other embodiments, a dose of about 700 μmoles/kg body weight of sodium hydroxide (NaOH) in a composition of 87.5 mM NaOH in saline is administered to an asthmatic subject by i.p. injection every other day for a total of 3 doses. Besides these specific examples, in general the embodiments follow the guidelines and principles set forth in the Definitions for the selection of pH modifiers, their doses, dosing regimens and routes and means of administration.

As demonstrated in the Examples, pH modifiers (especially but not exclusively those that decrease pH or resist the rise of pH) when used at the specified doses and dosing regimens can selectively deplete and suppress the population and proliferation of inflammatory and/or non-inflammatory pathological cells without overt damages to normal organs or tissues, for example the blood vessels, lung parenchyma, and airways. Thus, inflammatory cells are more susceptible to depletion by pH modifiers than tissue structural functional cells. In addition, the susceptibility of a cell to depletion and suppression of its proliferation by pH modifier is positively correlated with the cell's proliferative status, so that the more proliferative a cell is the more susceptible to depletion and suppression. The data disclosed in the Examples also show that some pH modifiers (e.g., those such as NaOH that increase pH) can increase the population and/or rate of proliferation of proliferating cells, while they also deplete inflammatory cells.

Further, the data disclosed in the Examples show that the effects of the pH modifiers on proliferating cells apply to all cell types. Such broad-spectrum effect is particularly relevant because pulmonary inflammation is often accompanied by hyperplasia of the structural functional cells of the lungs and airways (including but not limited to pneumocytes, goblet cells and fibroblasts). Moreover, lymphocyte proliferation in and outside the lungs (for example, in mediastinal lymph nodes) play crucial roles in both the initiation and the exacerbation of pulmonary inflammation. Therefore, these proliferating cells, inflammatory and non-inflammatory, are also pathological cells in the diseases of pulmonary inflammation. Selective depletion or suppression of such pathological cells and/or their proliferation by pH modifiers such as (but not exclusively) those the decrease pH or resist the rise of pH is beneficial to a subject suffering from or susceptible to pulmonary inflammation and/or allergic diseases.

In terms of concern about potential deletion or suppression of proliferating normal cells and/or their proliferation, it is important to note that in adults only a small number of normal cell types in a few organs (namely the thymus, bone marrow, skin and intestines) are actively proliferating. The experimental observations in the Examples show that the transient reduction of such small number of normal cells and/or their proliferation in the few organs is well tolerated by subjects treated with the pH modifiers.

Conversely, selective increase of protective normal cells and/or their proliferation by pH modifiers such as (but not exclusively) those that increase pH or resist the fall of pH can also be beneficial to a subject suffering from or susceptible to diseases of pulmonary inflammation. Such protective normal cells include (but may not be limited to) functional subsets of inflammatory cells with anti-inflammatory, immune suppressive activities, or both. Increasing the population and proliferation of these cells helps dampen the overzealous inflammatory and immune responses. In addition, structural functional cells of the damaged tissues or organs are also normal cells. Selective increase of the population and proliferation of such normal cells can facilitate the restoration of the damaged tissues or organs.

Another common pathological feature of pulmonary inflammation is mucus hypersecretion. As demonstrated in the Examples, in some embodiments, a composition comprising one or more pH modifiers (for example but not exclusively those that decrease pH or resist the rise of pH) is administered to a subject to selectively reduce or eliminate mucus hyper-secretion. Mucus hypersecretion is a major cause of airflow obstruction in both acute exacerbation of asthma and chronic severe asthma. (64). In other muco-obstructive lung diseases such as COPD, cystic fibrosis, primary ciliary dyskinesia, and non-cystic fibrosis bronchiectasis, inflammatory responses are also key to mucus hypersecretion. (For review see (65)). The suppression of mucus production by pH modifiers could be a direct effect on mucus-producing cells or indirectly through reduction of inflammatory cells. Regardless of the underlying mechanisms, the embodiments for reducing mucus production are applicable to asthma, other muco-obstructive lung diseases, and any other diseases, e.g., pneumonia, where mucus hypersecretion obstructs airflow.

Thus, the presently disclosed subject matter offers simultaneous relief from multiple pathological features and/or symptoms of pulmonary inflammation (i.e., inflammation, hyperplasia in the lungs and airways, and mucus hypersecretion).

In some embodiments, a composition comprising one or more pH modifiers is administered to a subject suffering from allergic diseases other than allergic asthma. These other allergic diseases may not affect the lungs. They include (but are not limited to) allergic rhinitis, allergic dermatitis, food allergy and urticaria. For example, a composition of about 5 mM to 16M HOAc in water can be topically applied, 4 times per day (about once every 4 hours) for two consecutive days, to lesions of allergic contact dermatitis caused by a subject's exposure to poison ivy to accelerate recovery from the disease.

### Treatments and/or Prevention of Inflammatory Diseases of the Blood, Blood Vessels

Inflammatory cells or leukocytes exist within the blood vessels in two ways—in suspension in the blood or in attachment to the blood vessels. Under certain clinical conditions (including but not limited to infections and allergy), inflammatory cells in the blood increase to above normal levels. On the other hand, while the attachment of inflammatory cells to the blood vessels is normally temporary, under some clinical conditions (e.g., atherosclerotic cardiovascular diseases (ASCVD)) inflammatory cells (especially but not exclusively monocytes/macrophages) become permanently attached to and invade the blood vessels, which together with other cells such as smooth muscle cells proliferate and ingest lipids to form atheromatous plaques. Moreover, under yet other conditions (e.g., vasculitis), the blood vessels are infiltrated by various inflammatory cells. These clinical conditions are collectively referred to as “inflammatory diseases of the blood, blood vessels” in this invention.

As demonstrated in the Examples, like in the lungs, inflammatory cells are effectively depleted in the blood by pH modifiers, whereas the structural integrity of the blood vessels is preserved. Thus, in part based on such experimental data, in some embodiments a composition comprising pH modifiers is administered to a subject suffering from or susceptible to inflammatory diseases of the blood, blood vessels. A composition comprising one or more pH modifiers especially but not exclusively those that decrease pH or resist the rise of pH is used for the treatment and/or prevention of such diseases by selectively decreasing pathological cells and/or their proliferation, wherein the pathological cells are the inflammatory and/or proliferating cells in the blood, inflamed blood vessel wall or the atheromatous plaques (e.g., macrophages and hyperplastic smooth muscle cells). Conversely, a composition comprising one or more pH modifiers especially but not exclusively those that increase pH or resist the fall of pH is administered into a subject to selectively increase protective normal cells and/or their proliferation, wherein the protective normal cells are functional subsets of inflammatory cells with anti-inflammatory, immune suppressive activities.

For examples, in one embodiment, a subject suffering from inflammation in the blood resulted from allergic asthma receives intratracheal instillation of a dose of about 420 μmoles/kg body weight of HOAc or HCl in a composition of about 175 mM HOAc or HCl in saline every other day for a total of 3 doses; in another embodiment, a subject suffering from inflammation in the blood resulted from allergic asthma receives intraperitoneal injection of a dose of about 700 μmoles/kg body weight of NaOH in a composition of about 87.5 mM NaOH in saline every other day for a total of 3 doses. Besides these specific examples, in general the embodiments follow the guidelines and principles set forth in the Definitions for the selection of pH modifiers, their doses, dosing regimens and routes and means of administration.

In some embodiments, a composition comprising pH modifier(s) is administered to a subject predisposed to ASCVD to prevent or slow the development of ASCVD through what is herein referred to as a “vessel cleansing” process (i.e., disrupting, diminishing, or removing the atheromatous plaques from the blood vessels by selectively depleting or suppressing the pathological cells and/or their proliferation). The selection of pH modifier(s), their doses, dosing regimens, and routes and means of administration follows the general guidelines and principles set forth in the Definition, and must also take into consideration of risk factors for ASCVD. The risk factors for ASCVD include (but are not limited to) age, hypertension, high cholesterol levels, diabetes mellitus, obesity, cigarette smoking, sedentarism, sleep deprivation, and/or psychosocial stress. (66-69). The presence of a single or any combination of the risk factors in a subject is the basis for considering the preventive treatment with pH modifiers, and also in part for deciding the choices of pH modifier(s), doses, dosing regimens, which should be made by the subject's attending health care provider(s) within the scope of sound medical judgment.

### Treatments and/or Prevention of Autoimmune Diseases

Inflammatory and/or immune responses to self-antigens are the cause of autoimmune diseases. Autoimmune diseases include (but are not limited to) multiple sclerosis, Anti-NMDA (N-methyl-D-aspartate) receptor encephalitis, autoimmune vasculitis, Giant cell myocarditis, Grave's disease, lupus, inflammatory bowel diseases, rheumatoid arthritis, and psoriasis. In some embodiments, a composition comprising one or more pH modifiers (especially but not exclusively those that decrease pH or resist the rise of pH), is administered to a subject suffering from or susceptible to autoimmune diseases to treat or prevent the diseases by selectively decreasing pathological cells and/or their proliferation; or conversely, a composition comprising one or more pH modifiers (especially but not exclusively those that increase pH or resist the fall of pH) is administered to the subject to selectively increase protective normal cells and/or their proliferation. In autoimmune diseases, the main pathological cells are the effector inflammatory cells directly or indirectly reactive to self-antigens, whereas the main protective normal cells are functional subsets of inflammatory cells with anti-inflammatory, immune suppressive activities.

For examples, in some embodiments a dose of about 700 μmoles/kg body weight of HOAc or HCl in a composition of 87.5 mM HOAc or HCl in saline is administered to a subject at an early stage of multiple sclerosis by intraperitoneal injection or infusion to blood or cerebrospinal fluid every other day for a total of 4 doses to stop the progression of the disease to advanced stages; in other embodiments, a dose of about 700 μmoles/kg body weight of HOAc in a composition of 87.5 mM HOAc in saline is administered to a subject at risk of multiple sclerosis but has not developed symptoms of the disease by intraperitoneal injection or infusion to blood or cerebrospinal fluid every other day for a total of 3 doses to prevent and/or delay the onset of the disease. In some embodiments, the time for the preventive treatments of multiple sclerosis is soon after a subject is diagnosed with radiologically isolated syndrome or clinically isolated syndrome, both of which are considered precursors to multiple sclerosis. (70, 71). In some embodiments, a composition of 5 mM-16M HOAc in water may be topically applied to lesions of psoriasis, 4 times a day, for therapy. Besides these specific examples, in general the embodiments follow the guidelines and principles set forth in the Definitions for the selection of pH modifiers, their doses, dosing regimens, concentrations in composition, and routes and means of administration.

While in most autoimmune diseases, inflammatory and/or immune responses to self-antigens impair the functions of the affected tissues and/or organs, the opposite is the case for some autoimmune diseases. For example, in Grave's disease, the thyroid gland is hyperactive due to stimulation of the thyrocytes by autoantibodies. Similar to hyperplasia of the lung cells in pulmonary inflammation, the thyrocytes in Grave's disease are hyper-proliferative or hyperplastic and excessively produce thyroid hormone. (72). Therefore, the pathological cells in such autoimmune diseases also include the hyperactive, hyperplastic tissue cells such as the hyperactive, hyperplastic thyrocytes in Grave's disease. Such hyperplastic pathological cells can also be selectively depleted by pH modifiers as compared with their less or non-proliferative normal counterparts because they are more proliferative.

### Treatment of Infectious Diseases

Although pulmonary and blood inflammation caused by infections have been alluded to in preceding sections, the presently disclosed subject matter is not limited to infections in the blood, lungs, and airways. Rather, the disclosed subject matter is applicable to infections at any anatomical location.

With regard to the treatments of infectious diseases, the embodiments of this invention offer new ways to achieve two distinct goals. One is to dampen overzealous inflammatory and/or immune responses, and the other is to promote protective inflammatory and/or immune responses. At a given time in the clinical course of an infectious disease, the embodiments focus on either of these two goals depending on whether the primary clinical concern is overzealous or insufficient inflammatory and/or immune responses. Thus, when the primary concern is overzealous inflammatory and/or immune responses, the major pathological cells are the effector inflammatory cells, whereas the major protective normal cells are the functional subsets of inflammatory cells with anti-inflammatory, immune suppressive activities. Other pathological cells may include but are not limited to host target cells. When the primary concern is insufficient protective immunity, effector inflammatory cells are the major protective normal cells, whereas functional subsets of inflammatory cells with anti-inflammatory, immune suppressive activities and host target cells are the main pathological cells. In some embodiments, a composition comprising one or more pH modifiers (especially but not exclusively those that decrease pH or resist the rise of pH) is administered to a subject suffering from one or more infectious diseases to selectively deplete and/or suppress the pathological cells and/or their proliferation. Conversely, a composition comprising one or more pH modifiers (especially but not exclusively those that increase pH or resist the fall of pH) is administered the subject to selectively increase protective normal cells and/or their proliferation.

For example, in some embodiments a dose of about 420 μmoles/kg body weight of HOAc is administered to a subject suffering from pneumonia by inhalation of a composition of aerosol comprising 175 mM HOAc in saline every other day for a total of 3 doses to dampen overzealous inflammatory response; in other embodiments a dose of 700 μmoles/kg body weight of NaOH is administered to a subject suffering from chronic lung infection by i.p. injection of a composition of 87.5 mM NaOH in saline every other day for a total of 3 doses to enhance anti-microbial effector inflammatory and/or immune responses. Besides these specific examples, in general the embodiments follow the guidelines and principles set forth in the Definitions for the selection of pH modifiers, their doses, dosing regimens, concentrations in composition, and routes and means of administration.

Another fundamental challenge of treating infectious diseases is to directly reduce or stop the proliferation and/or spread of the infectious agents in a subject, particularly the infectious agents that are resistant to other drugs (e.g., antibiotics). The presently disclosed subject matter offers a unique way of meeting this challenge. While not wishing to be bound to any particular theory, the data disclosed in the Examples showed that low pH induce cell death whereas high pH promotes proliferation. This explains the Warburg effect of tumor and normal proliferating cells, i.e, such cells switch from oxidative respiration to glycolysis for energy production. (73, 74). This is because oxidative respiration generates protons, thereby lowers pH. Therefore, the Warburg effect is necessary for the proliferating cells to create or maintain a relatively high pH for survival and proliferation. Importantly, the Warburg effect is not limited to mammalian cells, but is evolutionarily conserved. For examples, bacteria and yeasts switch to glycolysis when their proliferation and glucose supply reach critical levels. (75, 76). Likewise, parasites that cause parasitic diseases such as malaria, schistosomiasis, trypanosomiasis and leishmaniasis switch to glycolysis during the stages of their life cycles in mammalian hosts. (77-79). Therefore, like the effects on lymphocytes and tumor cells, pH modifiers, especially but not exclusively those that decrease pH or resist the rise of pH, can deplete and suppress infectious agents and their proliferation.

In addition, regardless of whether it is related to the Warburg effect or not, it is a fact that most pathogens, for examples E. coli, staphylococci, and Salmonella, require neutral pH for optimal proliferation. (80, 81). More generally, the proliferation of any pathogens regardless of their pH adaptive types and/or drug resistance abilities can be deterred or even stopped if the pH of their growth environment becomes less conducive to, or incompatible with, their propagation. However, this weakness of the pathogens has not been exploited for therapeutic use before largely because of the lack of appreciation of the medical implications of the body's tolerance and differential cellular responses to pH fluctuation. Thus, in some embodiments for the treatment of infectious diseases, pH modifiers, their doses and dosing regimen are selected to selectively decrease or stop the propagation and/or spread of pathogens in a subject by making the pH in or on the body of the infected subject and/or at the anatomical location of infection less conducive to or incompatible with the propagation of the pathogens.

The propagation of some pathogenic microbes (e.g., viruses) is dependent on their host target cells. Some of the pathogenic microbes, including but not limited to Epstein-Barr virus (EBV), human T-cell leukemia virus type I (HTLV-1), human papilloma virus (HPV), and Kaposi's sarcoma herpesvirus (KSHV), induce or enhance the proliferation of host target cells for the viruses' own replication. (82-86). Since the infected host target cells promote the pathogenesis of the infectious disease, they become pathological cells. For the treatment of such diseases, the presently disclosed subject matter is also particularly advantageous over other treatments because pH modifiers (especially but not exclusively those that decrease pH or resist the rise of pH) can selectively decrease the host target cells and/or their proliferation because of their increased rate of proliferation.

### Treatment and/or Prevention of Neoplasia

In some embodiments, a composition comprising one or more pH modifiers (especially but not exclusively those that decrease pH and/or resist the rise of pH) can be administered to a subject suffering from or susceptible to malignant or benign neoplastic diseases. The composition selectively depletes and/or suppresses the population and proliferation of neoplastic cells and other pathological cells such as functional subsets of inflammatory cells with anti-inflammatory, immune suppressive activities, thereby reduces or eliminates neoplastic growth and cancer metastasis.

While not wishing to be bound to a specific theory, the experimental data disclosed in the Examples of this invention showed that high pH promotes proliferation whereas low pH induced cell death in both lymphocytes and tumor cells. Therefore, a cellular pH balance controls both cell death and proliferation. This conclusion provides an explanation for the Warburg effect, i.e., tumor and normal proliferating cells switch from aerobic respiration to glycolysis for energy production. (73, 74). This is because aerobic respiration produces not only energy but also protons, thereby tips the pH balance lower. Therefore, tumor cells and other proliferating cells must tune down or stop aerobic respiration in order to create or maintain a high pH environment for both survival and proliferation. Since the Warburg effect is universal, embodiments similar to the Examples in this invention are applicable to control of cell death and proliferation of all tumor cells and other proliferating cells.

For examples, in some embodiments a subject suffering from lung cancer (small cell lung cancer, large cell carcinoma, adenocarcinoma or squamous cell carcinoma of the lungs) may receive intra-tracheal instillation or inhalation of a dose of about 420 μmoles/kg body weight of HOAc in a composition of about 175 mM HOAc in saline every other day for a total of three doses. In some embodiments, a subject suffering from acute lymphocytic leukemia or acute myelogenous leukemia may receive a dose of 700 μmoles/kg body weight of HOAc or HCl by i.p. injection of a composition of 87.5 mM HOAc or HCl in saline, or i.v. infusion of a composition of 50 mM HOAc or HCl in saline at a velocity of 0.525 mmoles (or 10.5 ml composition)/minute, every other day for a total of 3 doses. In some embodiments, a subject suffering from colorectal adenocarcinoma may receive a dose of 700 μmoles/kg body weight of HOAc or HCl by i.p. injection of a composition of 87.5 mM HOAc or HCl in saline every other day for a total of 3 doses. In some embodiments, a subject suffering from multiple myeloma or primary bone cancer may receive a dose of 700 μmoles/kg body weight of HOAc or HCl by i.p. injection of a composition of 87.5 mM HOAc or HCl in saline, or i.v. infusion of a composition of 50 mM HOAc or HCl in saline at a velocity of 0.525 mmoles (or 10.5 ml composition)/minute, every other day for a total of 3 doses. Besides these specific examples, in general the embodiments follow the guidelines and principles set forth in the Definitions for the selection of pH modifiers, their doses, dosing regimens, and routes and means of administration.

As shown in the Examples, the pH modifiers are well tolerated in a subject, and therefore are expected to have fewer or less severe adverse side effects than conventional chemotherapies, radiation therapies, and some new therapies such as immune therapies that often cause severe damages to normal tissues or cells. In addition, since the pH modifiers can permeate the entire body of a subject, in some embodiments the present invention is also particularly effective for preventing tumorigenesis and metastasis by selectively depleting cancer cells at the early stages of tumorigenesis and/or at the metastatic stages when the cancer cells are difficult or impossible to locate.

The presently disclosed subject matter contradicts a previous view that acidic environment is favorable for tumorigenesis and metastasis (87). Specifically, as demonstrated in the Examples, pH modifiers that decrease pH or resist the rise of pH deplete and suppress proliferating cells, including cancer cells, and/or the proliferation of such cells. For the same reason, the presently disclosed subject matter also distinguishes itself from the so-called “alkaline therapy” (i.e., consuming vegetable and fruit rich diets and beverages as an alternative therapy for cancers, presumably by neutralizing the acidic tumor microenvironment). Apart from this fundamental distinction, the presently disclosed subject matter comprises administering chemically defined compounds to a subject instead of consuming certain foods or beverages. Moreover, although alkaline therapy has gained some popularity in the mass media, a systemic review of both scientific and mass medial literatures concluded that there is little scientific evidence for the beneficial effect of alkaline therapy on cancer patients or that the tumor microenvironment could be altered by consuming the diets or beverages. (88).

In some embodiments, a composition comprising pH modifier(s) (especially but not exclusively those that increase pH or resist the fall of pH) is administered to a subject suffering from or susceptible to neoplastic disease to selectively increase the protective normal cells and/or their proliferation, instead of suppressing tumorigenesis or metastasis per se. The protective normal cells include (but are not limited to) effector inflammatory cells against the neoplastic cells. As shown in the Examples, pH modifier such as NaOH that raises pH increases the population and/or proliferation of immune cells and their precursors, therefore can promote inflammatory and/or immune responses against the neoplastic cells. For example, a dose of about 700 μmoles/kg body weight of NaOH is administered to a subject suffering from or susceptible to neoplastic disease by intra-peritoneal injection of a composition of about 87.5 mM NaOH in saline to enhance anti-neoplastic inflammatory and/or immune responses.

Besides these specific examples, in general the embodiments follow the guidelines and principles set forth in the Definitions for the selection of pH modifiers, their doses, dosing regimens, and routes and means of administration.

### Promoting Lymphopoiesis and/or Hematopoiesis

In some embodiments, a composition comprising pH modifier(s), especially but not exclusively those that increase pH and/or resist the fall of pH, is administered to a subject to selectively increase the generation of lymphocytes and/or blood cells (red and/or white blood cells). Such a subject may include but is not limited to a subject who has suffered from anemia, loss of blood due to trauma or surgery, or has received chemotherapy, and/or radiation therapy that reduce the genesis of lymphocytes and/or blood cells; someone who is the donor of cells of blood, bone marrow or stem cells for transplantation or adoptive cell transfer.

As an example, a dose of about 700 μmoles/kg body weight of NaOH is administered to a bone marrow donor by intra-peritoneal injection of a composition of about 87.5 mM NaOH in saline every other day for a total of 3 doses to selectively increase the populations and/or proliferation of white bone marrow cells. Besides this specific example, in general the embodiments follow the guidelines and principles set forth in the Definitions for the selection of pH modifiers, their doses, dosing regimens and routes of administration.

### Promoting Wound Healing and/or Tissue Regeneration

In some embodiments, a composition comprising one or more pH modifiers (including but not limited to those that increase pH and/or resist the fall of pH) is administered to a subject to selectively promote wound healing and/or tissue regeneration. For example, for a subject suffering from burned skin, a cream comprising one or more pH modifiers together with or without other medicines is topically applied to the damaged skin.

### Enhancement of Vaccine Efficacy

Vaccines have become powerful tools for combating an ever-increasing number of medical conditions, from the prevention of infectious diseases to the prevention and treatment of cancer, drug addiction, and more. (89, 90). As shown in the Examples, some pH modifiers (e.g., those that increase pH or resist the fall of pH) can promote draining lymph node cell proliferation, lymphopoiesis. and/or hematopoiesis, which increase the frequencies of antigen specific lymphocytes and antigen presenting cells. In part based such novel findings, in some embodiments, a composition comprising pH modifier(s), especially but not exclusively those that increase pH or resist the fall of pH, is administered to a subject at the same time as, before, or after vaccination, either alone or in combination with vaccine(s) to selectively enhance the efficacy of the vaccines for the treatment and/or prevention of the various medical conditions.

For example, in some embodiments, a subject receives intra-peritoneal injection of a dose of about 700 μmoles/kg body weight of NaOH in a composition of about 87.5 mM NaOH in saline every other day starting day 2 post vaccination for a total of 3 doses to selectively promote immune responses to the vaccines. Besides this example, in general the embodiments follow the guidelines and principles set forth in the Definitions for the selection of pH modifiers, their doses, dosing regimens, concentrations in compositions, and routes and means of administration.

In some subjects of vaccine recipients, pathological cell proliferation (e.g., neoplasia) is a significant risk. In addition, to achieve best therapeutic and/or preventive effects of the vaccines, certain types of immune responses to the vaccines may be preferred, e.g., antibody responses over cellular immune responses or vice versa, or immune responses dominated by certain functional subsets of immune cells. Under these circumstances, it is desirable to selectively increase the population and/or proliferation of protective normal (immune) cells responsive to the vaccines but not pathological cells or irrelevant normal cells, and/or selectively decrease pathological cells but not the protective normal (immune) cells and/or their proliferation. The present invention is particularly amenable to achieve such goals of shaping the protective immune responses by exploiting the differences of sensitivity and/or susceptibility to treatment with pH modifiers among different immune cells and/or functional subsets of immune cells.

For example, as demonstrated in the Examples, intra-tracheal instillation of HOAc increased the proportion of B cells relative to T cells in the lung draining lymph nodes. Therefore, as an example of embodiments, a subject of recipient of vaccine delivered via the airways may receive intra-tracheal instillation or inhalation of a dose of about 420 μmoles/kg body weight of HOAc in a composition of about 175 mM HOAc in saline on day 2 post vaccination to selectively increase B cells relative to the T cells in the draining lymph nodes to selectively promote antibody response over cellular immune response to the vaccines.

Besides the specific examples, in general the embodiments follow the guidelines and principles set forth in the Definitions for the selection of pH modifiers, their doses, dosing regimens, concentrations in compositions and routes and means of administration.

### Treatment of Overweight and Obesity

In some embodiments, a composition comprising one or more pH modifiers (especially but not exclusively those that decrease pH and/or resist the rise of pH) is administered to a subject suffering from or susceptible to overweight and/or obesity to selectively reduce body weight and/or fat mass. As demonstrated in the Examples, the injection of a composition of saline plus HCl or HOAc, and to lesser degree NaOH to an obese subject reduced body weight and fat masses. Thus, for example, in some embodiments a dose of about 700 μmoles/kg body weight of HCl or HOAc is administered to a subject suffering from obesity or overweight by intra-peritoneal injection of a composition of about 87.5 mM HCl or HOAc in saline every other day for a total of 4 doses to selectively reduce body weight and/or fat masses. Besides this specific example, in general the embodiments follow the guidelines and principles set forth in the Definitions for the selection of pH modifiers, their doses, dosing regimens, and routes and means of administration.

As demonstrated in the Examples, injection of saline plus HCl or HOAc reduces the population and/or rate of proliferation of proliferating cells in the epithelium of the intestinal villi and shortens the intestinal length. Based on this observation, overweight and obesity can also be reduced by inhibiting intestinal epithelial cell proliferation/renewal by cell proliferation inhibitors other than pH modifiers. Therefore, in some embodiments a composition comprising one or more cell proliferation inhibitors can be administered to a subject suffering from overweight or obesity to selectively decrease proliferating cells and/or their proliferation in the epithelium of the intestinal villi for the purpose of reducing body weight and/or fat mass. For example, a dose of about 0.5 mmoles/kg body weight of the epithelial growth factor receptor (EGFR) signaling inhibitor AG1478 is administered to a subject by intra-peritoneal injection of a composition of about 50 mM AG1478 in saline twice a day (about 12 hours apart). Besides this specific example, in general the embodiments follow the guidelines and principles set forth in the Definitions for the selection of the cell proliferation inhibitors, their doses, dosing regimens, concentrations in compositions, and routes and means of administration.

In some embodiments, one or more of both pH modifiers and cell proliferation inhibitors may be administered together or separately to the same subject to reduce overweight or obesity.

In some embodiments, surgical shortening of the intestines can be applied to a subject for the purpose of reducing body weight and/or fat mass. Such surgical procedure can be performed according to commonly adhered medical standards with or without robotic assistance. A segment of about 1% to 80% (e.g., 1%, 5%, 10%, 15%, 20%, 30%, etc.) of the small intestines may be resected from a subject in such embodiments.

The intestinal length is amenable to change under different physiological and pathophysiological conditions. It is often shortened when a subject is experiencing certain conditions such as colitis. Such shortening is usually reversible. However, even when a subject's intestines are irreversibly shortened, for example, by surgical removal of a segment of the intestines, a subject can adapt to the shortened intestines to avoid malnutrition. (91). Therefore, it is expected that a subject who has received intestinal resection or chemical shortening of intestinal length by pH modifiers and/or cell proliferation inhibitors as in this invention would not suffer from malnutrition. Indeed, in the Examples disclosed in this invention, subjects that had received acid treatments did not show signs of malnutrition other than the reduction of fat/body weight ratio.

### EXAMPLES

The following Examples provide illustrative embodiments. In light of the present disclosure and the general level of skill in the art, those of ordinary skill in the art will appreciate that the following Examples are intended to be exemplary only and that changes, modifications, and alterations can be employed without departing from the scope of the presently disclosed subject matter. It is noted that much of the disclosed experimental data is derived from mouse studies. It should further be noted that the outcomes of the Examples do not preclude the possibility of different outcomes if different pH modifiers, doses, dosing regimens and subjects were to be used in similar procedures.

It is further noted that the Examples represent a series of experimental studies stemmed from a serendipitous discovery that an acid solution used to prepare a plant extract depleted inflammatory cells in the bronchoalveolar lavage fluid when instilled into the lungs of mice without causing notable extra stress to the mice.

### Materials and Methods

Unless specified otherwise, the materials and experimental methods used in all Examples disclosed herein are described as follows:

### Mice and Animal Models of Diseases

Female Balb/c and C57BL/6 mice were purchased from Jackson Laboratory (Bar Harbor, Me., USA). Mice were housed in the animal facility of Charles River Accelerator and Development Lab (CRADL) (Cambridge, Mass., USA). Animal studies were performed according to the protocols approved by the CRADL Institutional Animal Care and Use Committee (IACUC).

Asthma induction and analyses were performed essentially as previously described. (92, 93). Seven weeks old Balb/c mice from Jackson Laboratory were acclimated at CRADL for 1 week. For sensitization, grade II ovalbumin (OVA) (Sigma-Aldrich, St. Louis, Mo., USA) and Alum adjuvant (Thermo Scientific, Rockford, Ill., USA) were fresh mixed in PBS to make sensitization solution comprising 200 μg/ml OVA and 33.4% (by volume) Alum. The Balb/c mice were i.p. (intraperitoneally) injected with 100 μl of the sensitization solution (20 μg OVA/mouse) on day 0. On day 14, each mouse was sensitized again by i.p. injection of 500 μl of fresh prepared sensitization solution (100 μg OVA/mouse). Eighteen days later, mice were challenged with OVA together with or without pH modifiers as described later. For challenge, 2× concentrated solution was prepared by mixing 100 μg OVA protein and 1 μg OVA323-39 peptide (Molecular Resources, Fort Collins, Colo., USA) in 30 μl saline per mouse, and kept on ice. Immediately before i.t. (intratracheal) injection the 30 μl 2×OVA solution was mixed with equal volume of 2× treatment solution (350 mM HOAc or HCl in saline) and instilled into the trachea of the mouse. The mice were challenged every other day for a total of 3 times. On day 3 after the final challenge, each mouse was sacrificed individually by CO₂ inhalation. Blood, bronchoalveolar lavage fluid (BALF), lungs and lymphoid tissues were collected in such order for each mouse immediately following sacrifice.

Induction of experimental autoimmune encephalomyelitis (EAE), the mouse model of multiple sclerosis, was carried out as previously described. (94). (Also see https://hookelabs.com/protocols/eaeAI_C57BL6.html). Antigen emulsion was prepared by mixing myelin oligodendrocyte glycoprotein peptide MOG35-55 (G. L. Biochem, Shanghai, China), desiccated M. tuberculosis H37Ra (Difco Laboratories, Detroit, Mich., USA) and incomplete Freund's adjuvant (IFA) (Difco Laboratories) in PBS. The final antigen emulsion comprised 1.5 mg/ml of MOG35-55, 189 μg/ml of M. tuberculosis H37Ra and 50% (by volume) IFA.

Female C57BL/6 mice of 9 weeks old were acclimated at CRDAL for 1 week. On day 0, mice were immunized by s.c. (subcutaneous) injection of the antigen emulsion at 4 evenly distributed sites in the dorsal area (25 μl/site). Two hours later, the mice were i.p. injected with Pertussis Toxin (List Biological laboratories, Campbell, Calif., USA) freshly prepared in ice-cold PBS (200 ng in 100 μl PBS/mouse). On day 1, the mice received a second i.p. injection of Pertussis Toxin at the same dosage. Starting on day 10, at which time no disease onset was observed, EAE clinical scores of the mice were recorded using the scoring system described in Table 1 below. Mice with clinical score of 3 or above were provided wet chows and hydrogels.

TABLE 1
Definition of EAE clinical scores

| Score | Clinical phenotype |
|---|---|
| 0 | Tail is normal with full strength |
| 0.5 | Mild tail weakness |
| 1 | Whole tail weakness; when laid on a flat surface on the back, can flip up very |
| | well |
| 1.5 | When laid on a flat surface on the back, can't flip up very well |
| 2 | When laid on a flat surface on the back, can hardly flip up; can stand on a metal |
| | grid very well; can climb into the cage from the grid very well (within 3 seconds) |
| 2.5 | Can stand on the metal grid, but occasionally hind legs fall from the grid; has |
| | difficulty climbing into the cage from the grid (needs 3-10 seconds) |
| 3 | Can hardly stand on the grid, most of the time the legs fall through the grid; can |
| | hardly climb into the cage from the grid (needs more than 10 seconds); hind legs |
| | are weak but still movable |
| 3.5 | One hind leg is paralyzed; front legs are fine; ability to ambulate not affected |
| 4 | Both hind legs are paralyzed, but front legs are fine; the whole body can move |
| | well |
| 4.5 | Hind legs are paralyzed, front legs are weak; whole body ambulates with |
| | difficulty |
| 5 | Hind legs are paralyzed, front legs are very weak; mouse rarely moves |
| 6 | Death |

The obesity model was similar to those previously described. (95, 96). Female C57BL/6 mice of 4 weeks age were acclimated at CRADL for 1 week. After the acclimation, mice were switched to Gamma-irradiated high fat diet (Research Diets, New Brunswick, N.J., USA) (60% calories from fat). After 4 weeks of high fat diet, the mice were switched back to regular diet, and treated with i.p. injection of saline or saline plus HCl or HOAc. The bodyweight of each mouse was recorded before each treatment, and at the end of the experiment.

### In Vivo Treatments of Mice with Saline or Saline Plus pH Modifiers

For all examples, treatments of the mice were carried out either by i.t. (intra-tracheal) instillation or i.p. injection. For i.t. instillation, 60 μl of treatment solution of saline or saline plus 175 mM HCl or HOAc was instilled into the trachea of a mouse using a pipet tip. For i.p. injection, 200 μl of saline or saline plus 87.5 mM HCl, HOAc or NaOH was injected into the peritoneal cavity of a mouse using a 26 or 27G needle. At the time of the first treatment the body weights of the mice ranged about 19 to 30 g.

Unless stated otherwise, in the asthma model in Example 1, i.t. treatments were carried out by mixing 30 μl 2× concentrated treatment solution with 30 μl 2× concentrated OVA challenging solution per mouse. The total mixture (60 μl) was instilled into the trachea of the mouse. The mice were treated every other day for a total of 3 times. I.p. treatments in the asthma model followed the same schedule and were performed 1 hour after the i.t. instillation of 60 μl of 1×OVA challenging solution.

For the analyses of the effects of pH modifiers on lymphocyte populations and their proliferation in vivo in Example 3, the mice were sensitized, challenged with OVA and received either i.t. or i.p. treatments. The same challenge and treatment solutions as in the asthma model were used. In the first set of experiments, the mice were challenged with 60 μl of 1×OVA challenge solution without pH modifiers by i.t. injection for 2 consecutive days. One day of the second challenge, the mice received one-time i.t. treatment with saline or saline plus HCl or HOAc. The mice were sacrificed the next day by CO₂ inhalation to collect lymphoid tissues. In the second set of experiments, the mice were challenged with OVA and treated with i.t. instillation of saline or saline plus HCl or HOAc or treated with i.p. injection of saline plus NaOH for a total of 3 doses as in the asthma model. On day 3 after the final challenge and treatment, mice were sacrificed by CO₂ inhalation to collect lymphoid tissues.

In the EAE model of Example 2, two types of experiments, i.e., therapeutic and preventive experiments, were carried out. For the therapeutic experiments, on the day when the mice first reached the clinical scores of 1.5 to 2.5, the mice received i.p. treatments with saline, or saline plus 87.5 mM HCl or HOAc. The mice received additional 3 i.p. treatments every other day. For the preventive experiments, on day 11 after immunization when no onset of disease was detected, the mice received i.p. treatments with saline or saline plus 87.5 mM HOAc. The mice received i.p. treatments every other day for additional 2 doses. Clinical scores of all mice were recorded daily during and after the treatment periods, but experimentation was terminated on mice that reached clinical score of 5. As specified in some experiments, mice were sacrificed, and spinal cords were collected for histology analyses.

In the obesity model of Example 10, after fed with high fat diet for 4 weeks, mice were weighed to record total body weight. The mice were switched to regular diet and randomly divided into groups to receive i.p. treatments with saline or saline plus HCl, HOAc or NaOH. The mice continued to receive i.p. treatments on every other day for 3 additional doses. Four days after the final treatments, mice were sacrificed, their total body weights were measured, fat pads, and internal organs were collected, weighed, and analyzed.

### BALF Cellularity and Cell Differentials

In the asthma model of Example 1, on day 3 after the final OVA challenge and treatment with or without pH modifiers, mice were sacrificed. BALF were collected using a 1 cc syringe to inject and retreat 0.6 ml ice cold PBS plus 1% FBS from the trachea into and from the lungs for 3 times. The cells in the BALF of each mouse were stained with Trypan Blue and counted to determine total cellularity of the BALF. The BALF cells were spun onto glass slides using a StatSpin Cytofuge (Iris International, Westwood, Mass., USA). The slides were stained with hematoxylin and eosin (H&E) using the Diff-Quick Stain Set (Siemens Healthcare Diagnostics, Newark, Del., USA). Eosinophils, macrophages, lymphocytes, and neutrophils were counted under a light microscope with mounted camera, and images were taken and processed with Motic Image Plus 2.0 software (Swift Optical Instruments, San Antonio, Tex., USA).

### Blood Collection and Removal of Red Blood Cells

After the mice were sacrificed, 200 μl blood was drawn from the heart with a 26G needle attached to a 1 cc syringe, and immediately transferred to a microcentrifuge tube with 800 μl Hank's Balanced Salt Solution (HBSS) plus 1 mM EDTA. After centrifuge at 3000 rpm for 1 minute in a microcentrifuge, red blood cells were removed by resuspending the cells in 500 μl ddH₂O for 15 seconds, followed by addition of 500 μl 2× PBS. The cells were then centrifuged and resuspended in 1×PBS plus 1% FBS and kept on ice for further analyses.

### Analyses of Tissue Inflammatory Infiltration and Mucus Secretion

Lungs (after BALF collection), spinal cords and intestines were harvested and fixed in 10% neural formalin (Fisher Scientific, Kalamazoo, Mich., USA). The fixed lungs, spinal cords and segments of small intestines were paraffin embedded and sectioned on a Microtome (Leica Biosystem, Buffalo Grove, Ill., USA). The tissue sections were stained with hematoxylin and eosin (H&E) to determine inflammatory infiltration. Periodic acid-Schiff (PAS) staining was also performed with the lung tissue sections to determine mucus secretion in the lungs as a result of the inflammatory infiltration. The tissue slides were examined under a light microscope with mounted camera, and images were taken and processed with Motic Image Plus 2.0 software (Swift Optical Instruments, San Antonio, Tex., USA), or alternatively the slides were scanned and images were processed with the open source QuPath software (https://qupath.github.io).

### Immunohistochemistry Staining of Ki-67

Proliferating cells in the lungs and intestines were analyzed by immunohistochemical staining of Ki-67. Lungs and segments of jejunums were fixed in neutral formalin, paraffin embedded and sectioned. Immuno-staining of the tissue sections and chromogenic procedures were carried out as previously described. (97). Briefly, the tissue sections were first deparaffinized and rehydrated. Antigens in the tissue sections were unmasked by heating in 10 mM Sodium Citrate Buffer, pH 6.0, to boiling for 2 minutes in a microwave oven 3 times, followed by washing with PBS and incubation in 10% H₂O₂ at room temperature for 15 minutes. After washing with PBS, the tissue sections were blocked with 5% BSA. Afterwards, the tissue sections on the slides were incubated with rabbit anti-mouse Ki-67 antibody (GB13030-2, Servicebio, Wuhan, China) in PBS at 4° C. overnight. After washing with PBST (PBS plus 0.1% Tween 20), the tissue sections were incubated with Horseradish Peroxidase (HRP)-conjugated goat anti-rabbit antibodies (G21234, Invitrogen, Carlsbad, Calif., USA) at room temperature for 1 hr. After washing the slides, color deposits were developed using the DAB Substrate Kit (Cat. 34002, ThermoFisher, Waltham, Mass., USA) according to the manufacturer's instruction. After color was developed, the slides were washed with ddH₂O, and counter-stained with hematoxylin. The slides were scanned, and images were processed with the open source QuPath software (https://qupath.github.io).

### Culture of Tumor Cells

Jurkat and Raji tumor cell lines were maintained in Complete RPMI-1640 Medium (RPMI-1640 plus 1× GlutaMAX, 100 U/ml Pen-Strep (Gibco Life Technologies, Grand Island, N.Y., USA), and 5% heat-inactivated fetal bovine serum (FBS) (Atlanta Biologicals, Flowery Branch, Ga., USA)) at 37° C. and 5% CO₂. The tumor cells were washed 3 times with saline plus 1% FBS before in vitro treatments with saline or saline plus pH modifiers.

### In Vitro Treatments of Lymphocytes and Tumor Cells

For studies of the effects of pH modifier on proliferating cells, primary thymocytes or lymph node cells (4×10⁶ cells/ml) were incubated in FBS comprising 10% of saline or 87.5 mM HCl, HOAc or NaOH prepared in saline in 37° C. water bath for 5 hours before analyses. The tumor cells (2×10⁶ cells/ml) were incubated in FBS comprising 10% saline or 87.5 mM HCl, HOAc, or NaOH prepared in saline in 37° C. water bath for 3-5 hours.

### Flow Cytometry

The following conjugated anti-mouse antibodies and reagents were purchased from eBioscience/ThermoFisher Scientific (San Diego, Calif., USA): CD4-APC, CD4-eFlour 780, CD4-PE Cy7, CD8a-PE, CD44-eFlour 610, CD69-APC, Foxp3-FITC, Anexin V-PE, Avidin-APC-Cy7, 7AAD, CytoFix/CytoPerm buffer set. The following conjugated anti-mouse antibodies were purchased from Biolegend (San Diego, Calif., USA): CD8a-APC/Fire™ 750, F4/80-Pacific Blue, Siglec F-Biotin, Ly6G-Biotin, CD19-APC, Ki-67-PE, Ki-67-PE-Cy7, B220-Pacific Blue, Zombie-Green fixable viability kit, Zombie-Violet fixable viability kit; Foxp3 staining buffer set and PE-conjugated anti-human Ki-67 antibody. The following conjugated anti-mouse antibodies and reagents were purchased from BD Pharmingen/ThermoFisher Scientific (San Diego, Calif., USA): CD4-FITC, Avidin-PE, Avidin-PE-Cy5 and PE-conjugated anti-human Ki-67 antibody.

Cells were washed 3 times with PBS then stained with Zombie viability dyes at room temperature for 20 minutes. At the end of incubation, 10% volume of FBS was added to absorb excess Zombie dyes. After washes, the cells were stained with conjugated antibodies against surface antigens on ice. As specified in some experiments, the cells were subsequently stained with fluorochrome-conjugated Avidin. For Annexin V staining, cells were washed twice with plain PBS and once with Annexin V binging buffer (10 mM HEPES, pH7.4, 140 mM NaCl, 2.5 mM CaCl₂)) after surface antigen staining, and incubated with fluorochrome-conjugated Annexin V in Annexin V binding buffer for 15 min at room temperature. The cells were washed twice in Annexin V binding buffer and resuspended in Annexin V binding buffer. 7-AAD was added to the cells before analyzed by flow cytometry. For Ki-67 and Foxp3 staining, lymphocytes (after Zombie dye and surface antigen staining) or tumor cells (without surface antigen staining) were fixed and permeabilized using Foxp3 staining buffer kit, then stained with PE-conjugated anti-mouse or human Ki-67 antibodies. (Biolegend, San Diego, Calif., USA). Stained and unstained cells were analyzed on Accuri C6 Flow Cytomer (Accuri Cytometers, Inc., Ann Arbor, Mich.), FACS Canto (BD Bioscience, San Jose, Calif., USA) and Attune™ cytometer (Invitrogen, Carlsbad, Calif.). Data analyses were carried out using FlowJo X.

### Measuring Cell Division by CFSE Dilution

Peripheral lymphocytes (lymph node and spleen cells) were washed 3 times with plain PBS. After the washes, the cells (3-5×10⁶/ml) were incubated in 1 μM Carboxyfluorescein succinimidyl ester (CFSE) (Fluka/Sigma-Aldrich, Burlington, Vt.) in plain PBS at room temperature for 7 minutes. After the incubation, ¼ volume of FBS was added to stop the labeling, and cells were washed 4 times with PBS plus 1% FBS. The labeled cells were cultured under different conditions as indicated in specific experiments. Cell division, i.e., the dilution of the CFSE signals, was measured by flow cytometry.

### Measuring Intracellular pH

Intracellular pH detection pack comprising the pH indicators pHrodo™ Green AM and pHrodo™ Red AM and the PowerLoad was purchased from ThermoFisher Scientific (Waltham, Mass.). Staining of cells with either of the indicators was performed according to the manufacture's instruction. Briefly, lymphocytes were washed once with Live Cell Image Solution (LCIS) (Life Technology/ThermoFisher Scientific, Grand Island, N.Y.). Immediately prior to use, the pH indicator and the PowerLoad were mixed then diluted in LCIS to produce working solution comprising 0.5-1 μM pH indicator. The cells (1-2×10⁷/ml) were suspended in 0.5 ml working solution and incubated in a 37° C. water bath for 30 minutes. After the incubation, the cells were washed once in LCIS comprising 1% FBS. The fluorescence emitted by the pH indicators from the cells was detected by flow cytometry.

### Example 1

### Reduction of Pulmonary and Blood Inflammation by pH Modifiers

The mouse model of allergic asthma was used to show an example of depleting inflammatory cells in the lungs and blood by modest and transient disturbance of pH homeostasis with pH modifiers. Asthma was induced in Balb/c mice by sensitization with the experimental allergen OVA mixed with Alum adjuvant followed by challenging with OVA. The challenged mice were treated by i.t. instillation of saline or saline plus HCl, HOAc, or i.p. injection of saline plus NaOH. NaOH treatment was carried out by i.p. injection because mice were poorly tolerant of i.t. instillation of NaOH. However, this should not be construed as that the current invention excludes i.t. instillation as a route of administration of NaOH or other alkaline pH modifiers in humans or other animals because tolerance may vary among different subjects and at different doses or concentrations of the pH modifiers.

### Reduction of Inflammatory Cells in the Bronchoalveolar Lavage Fluids (BALF)

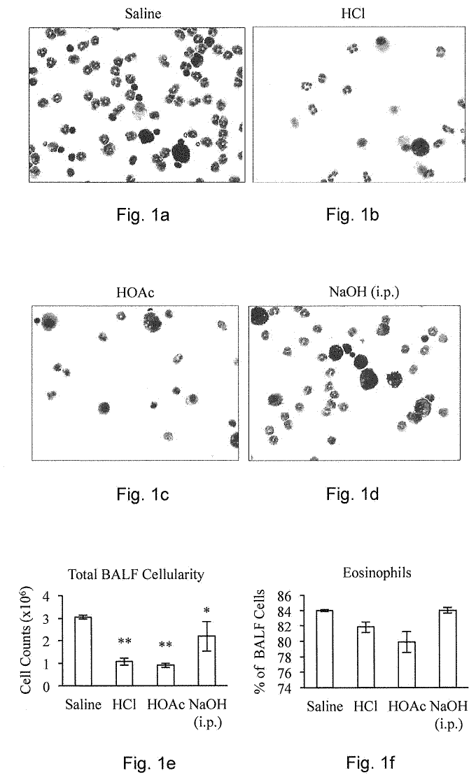

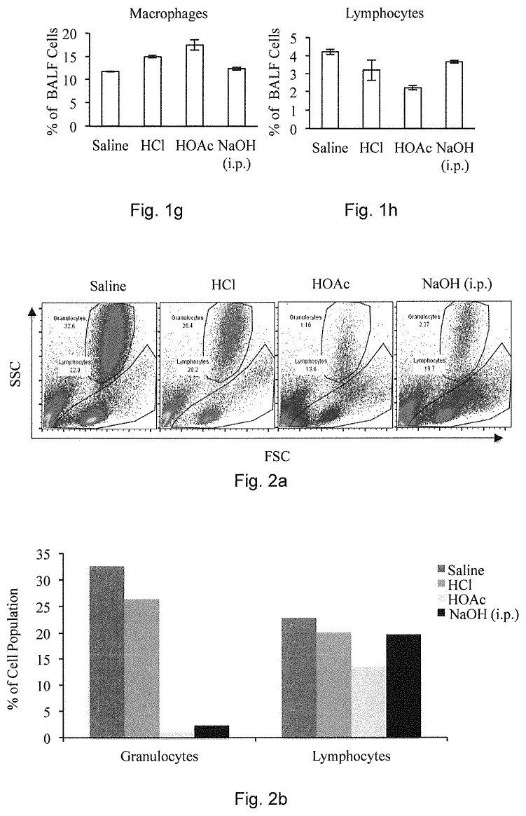

Inflammatory cells in the lumens of the lungs were first analyzed. BALF from the control saline-treated mice comprised large numbers of inflammatory cells (average of 3.06×10⁶/mouse). In contrast, the total inflammatory cells in the BALF of HCl- or HOAc-treated mice were dramatically reduced (averages of 1.07×10⁶ and 9.01×10⁵ per mouse, respectively). The total inflammatory cells in the BALF of the NaOH-treated mice (average of 2.18×10⁶/mouse) were also reduced, but to a much lesser degrees than the acid-treated mice. (FIGS. 1a-1e).

Despite the significant reduction of BALF cellularity by the pH modifiers, eosinophils remained as the predominant inflammatory cells in all treatment groups, ranging from 80-84% of total BALF cells. (FIG. 1f). The percentages of macrophages and lymphocytes also showed only small variations among the treatment groups, ranging from 12.3-17.5% and 2.2-4.2% of total BALF cells, respectively. (FIGS. 1g and 1h).

### Reduction of Inflammatory Cells in the Blood

The blood samples from the mice were analyzed by flow cytometry. The two distinct inflammatory cell populations of granulocytes and lymphocytes were defined by their FSC and SSC profiles. As shown in FIGS. 2a and 2b, the percentages of both of the inflammatory cell populations were reduced in mice treated with the pH modifiers as compared with the saline-treated mice. The treatment with HOAc caused the greatest reductions in these inflammatory cell populations as compared with the saline-treated mice (1.1% vs. 32.6% for granulocytes; 13.6% vs. 22.9% for lymphocytes). Such reduction of circulating inflammatory cells in the blood contributed in part to the reduction of inflammation in the lungs.

### Reduction of Inflammatory Infiltration in the Lung Tissues

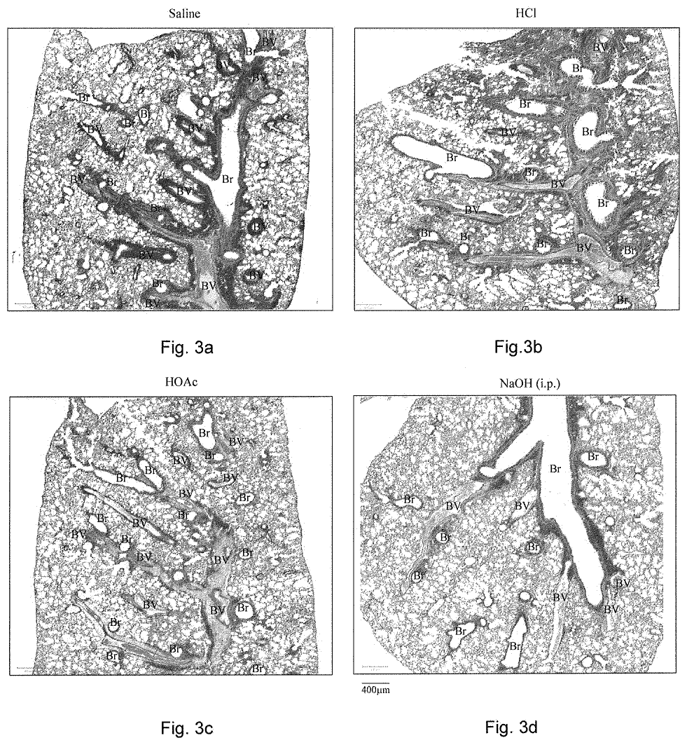

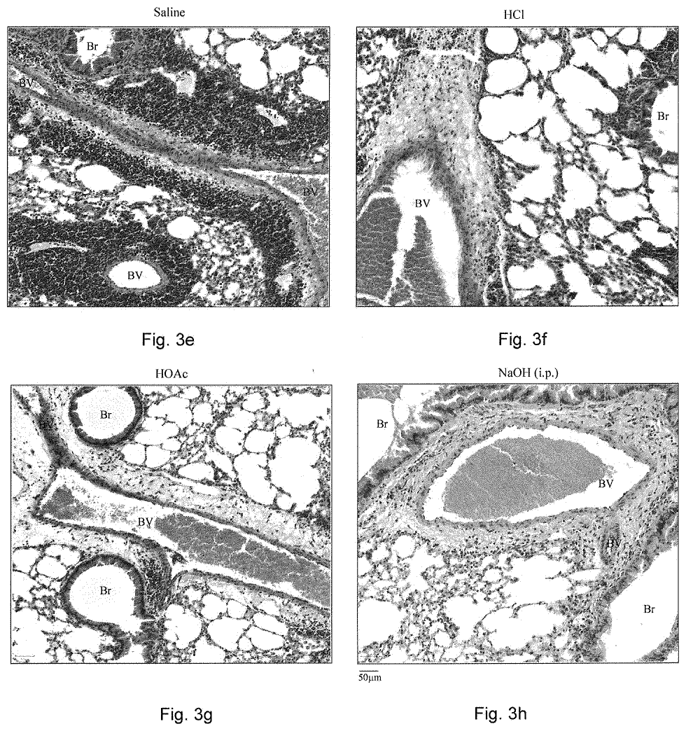

Next, inflammation in the lung tissues was examined. H&E staining showed that in the lungs of the saline-treated mice inflammatory infiltration was mostly concentrated in the perivascular and peribronchial areas but had also spread to other parenchymal spaces. (FIG. 3a). In the lungs of the mice i.t. treated with HOAc or HCl, inflammatory infiltration was diminished in both the perivascular and peribronchial areas, as well as other parenchymal spaces. The perivascular areas of the acid-treated mice showed a characteristic “faded” blue staining, which indicated that most of inflammatory cells that previously occupied the areas were depleted, and only residual inflammatory cells remained. (FIGS. 3b and 3c). In the lungs of the mice i.p. treated with NaOH, reduction of inflammatory infiltration was prominent in the perivascular areas, but not very noticeable in the peribronchial areas. (FIG. 3d). The difference might be attributed to the i.p. route of injection that caused the transportation of the injected NaOH to the lung tissues through blood circulation but not bronchial absorption. Microscopic examination of the lung tissues at high magnification further revealed that the structures of the blood vessels and the nearby alveoli and bronchioles were well preserved while inflammatory infiltration was effectively depleted by the pH modifiers. (FIGS. 3e-3h).

Thus, treatments with pH modifiers, reduced pulmonary inflammation not only by reducing circulating inflammatory cells in the blood, but also by directly depleting inflammatory cells in situ in the lung tissues without overt damages of the blood vessels, alveoli, and bronchioles.

### Inhibition of Mucus Hyper-Secretion in the Lungs

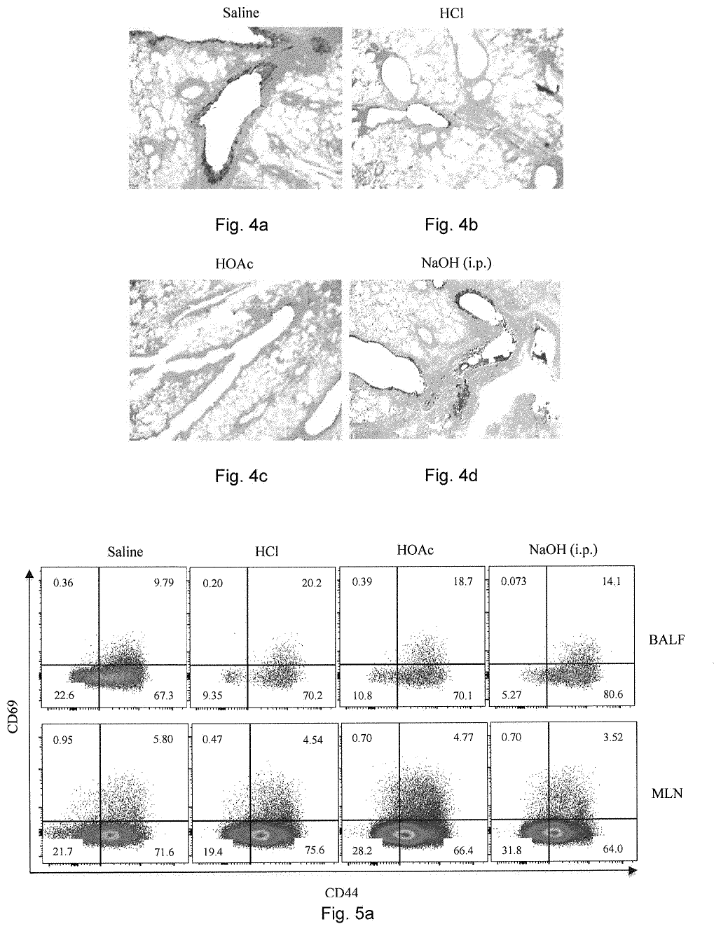

Mucus hyper-secretion in the lungs is a cardinal feature of pulmonary inflammation in allergic asthma. As expected, lungs of the control saline-treated mice showed heavy mucus secretion as indicated by the intense Periodic acid-Schiff (PAS) staining. (FIG. 4a). In contrast, lungs of the HOAc- or HCl-treated mice showed only faint PAS staining. (FIGS. 4b and 4c). However, no reduction of mucus secretion or PAS staining was observed in the lungs of the NaOH-treated mice. (FIG. 4d).

### No Inhibition of CD4 T Cell Activation in BALF and MLN

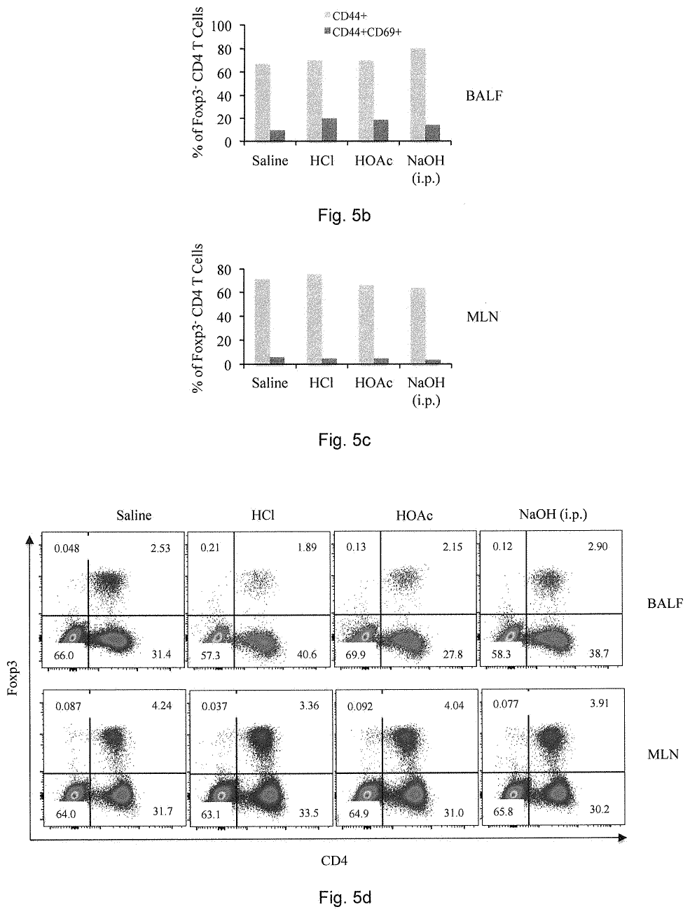

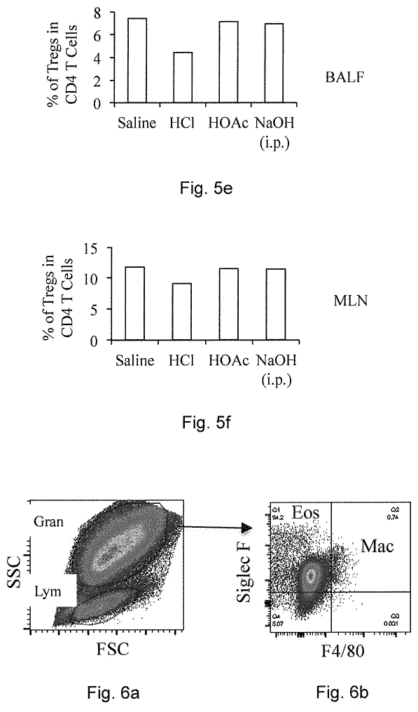

Pulmonary inflammation in allergic asthma results from the activation of CD4 T cells by allergens. (93). CD4 T cell activation was demonstrated by their expression of the activation marker CD44 and CD69. Most of the Foxp3⁻ CD4 T cells in the BALFs and the lung-draining mediastinal lymph nodes (MLNs) were CD44⁺, some of which co-expressed the early activation marker CD69, (FIGS. 5a-5c), indicating that the CD4 T cells in the BALFs and MLNs were predominantly effector or memory CD4 T cells.

The percentages of CD44⁺ CD4 T cells in the BALFs were comparable among saline- and acid-treated mice (67-70.2%), but higher in the NaOH-treated mice (80.6%). Notably, the percentages of CD44⁺CD69⁺ cells were higher in mice treated with the pH modifiers than that of the control mice treated with saline (14.1-20.2% vs. 9.79%), particularly in the acid-treated mice. (FIGS. 5a, 5b). This result suggests that the BALFs of the acid-treated mice were being replenished with newly activated CD4 T cells after the deletion of the T cells. In contrast, the percentages of CD44⁺ and CD44⁺CD69⁺ of Foxp3⁻ CD4 T cells in the MLNs, were less variable among the different treatment groups, in the ranges of 64-75.6% and 3.52-5.8%, respectively. (FIGS. 5a, 5c).

### No Increase of Treg Cell Populations in BALF and MLN

Regulatory T (Treg) cells inhibit T cell activation therefore could prevent pulmonary inflammation in allergic asthma. (98). Therefore, whether the Treg cell population was affected by the pH modifiers was examined. Treg cells in the BALFs of HCl-, HOAc- and NaOH-treated mice were 4.45%, 7.18% and 6.97% of total CD4 T cells, respectively, compared with 7.46% of the saline-treated mice. (FIGS. 5d, 5e). In the MLNs of HCl-, HOAc- and NaOH-treated mice, Treg cells were 9.12%, 11.53% and 11.46% of total CD4 T cells vs. 11.80% of the saline-treated mice. (FIGS. 5d, 5f).

Therefore, treatments with pH modifiers did not increase Treg cells, demonstrating that the reduction of pulmonary inflammation by pH modifiers was not due to increase of Treg cells. It should be noted that the finding that acid treatments did not increase Treg cells is in stark contrast to previous studies, which showed that some short chain fatty acids (SCFAs) can promote extra-thymic generation of Treg cells from peripheral naïve CD4 T cells. (99, 100). The discrepancy was due to the fact that the acid treatments in the current study occurred during recall response, i.e., in the allergen challenge phase, instead of during the activation of naïve CD4 T cells in the previous studies. Since the CD4 T cells in the recall responses were already committed to effector/memory cells, they were unlikely to be diverted to Treg cell differentiation.

### Induction of Death of Inflammatory Cells by Apoptosis

Having excluded the inhibition of CD4 T cell activation and induction of Treg cells as potential modes of action of the acid treatments for the depletion of inflammatory cells, whether induction of cell death was a potential mode of action was investigated.

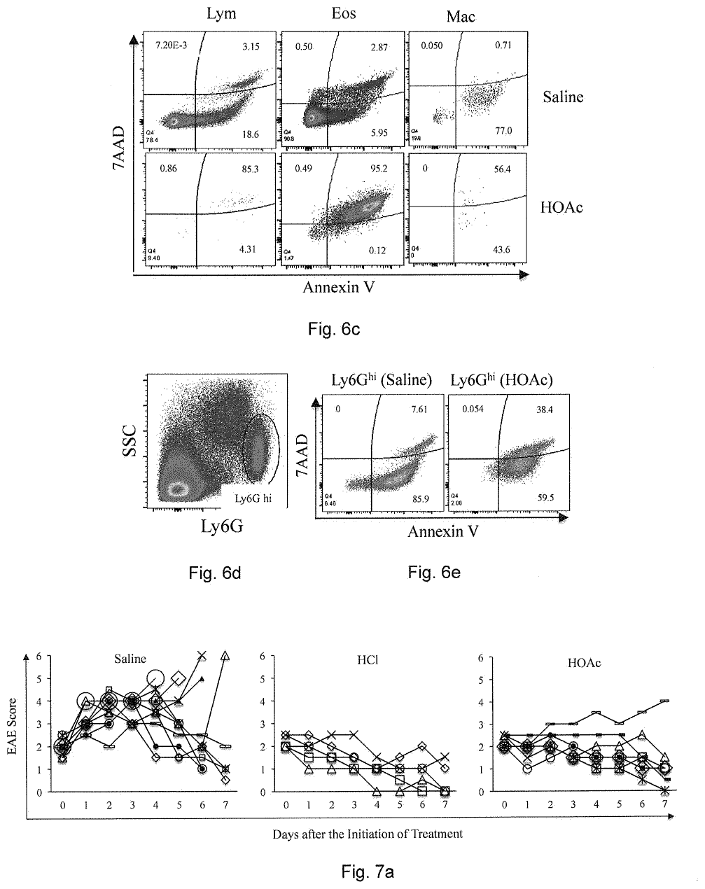

To investigate this possibility, OVA sensitized mice were challenged with OVA only. After the 3^{rd} challenge, the mice received either i.t. instillation of saline alone or saline plus HOAc. Shortly after the treatments, BALF cells were collected and stained for the eosinophil marker Siglec F, the macrophage marker F4/80, Annexin V and 7-AAD. Within the granulocyte populations of the BALF, eosinophils were Siglec F^{low-high}F4/80^{neg-low} whereas macrophages were Siglec F^{low}F4/80^{high}. (FIGS. 6a, 6b). In the saline-treated mice, only small fractions of the lymphocytes (3.15%), eosinophil (2.87%) and macrophages (0.71%) were apoptotic (Annexin V⁺7-AAD⁺). In contrast, the majorities of these cell populations of the HOAc-treated mice were apoptotic (85.3%, 95.2% and 56.4%, respectively). (FIG. 6c).

Neutrophils are another type of inflammatory cells. However, neutrophils were scarce in the BALF of mice in the asthma model. Therefore, blood samples were analyzed for the effects of HOAc treatment on neutrophils. Neutrophils were defined as Ly6G^{high} leukocytes in the blood. (FIG. 6d). (101, 102). In the blood of saline-treated mice, 7.61% of the neutrophils were apoptotic, whereas it was 38.4% in the blood of HOAc-treated mice. (FIG. 6e).

In summary, it was determined that treatment with pH modifiers can effectively reduce or abolish pulmonary and blood inflammation. Treatments with pH modifiers that decrease pH such as inorganic or organic acid (e.g., HCl and HOAc) also greatly reduced mucus hyper-secretion, whereas treatment with NaOH did not. This may be due to the fact that the NaOH was administered by i.p. injection, therefore did not reach the peribronchial spaces in sufficient amount. However, by choosing different pH modifiers and/or the concentrations of the pH modifiers in a composition, it may be possible to administer pH modifiers that increase pH or resist the fall of pH directly to the lungs to reduce mucus secretion.

### Example 2

### Depleting Inflammatory Cells in the Nervous Tissues by pH Modifiers

Example 1 demonstrated the depletion of inflammatory cells by pH modifiers, particularly the acids, in the course of allergic reactions in the lungs and airways. Example 2 will show that acid treatments can also deplete inflammatory cells in the nervous tissues under autoimmune condition. For this example, C57BL/6 mice were immunized with the myelin oligodendrocyte glycoprotein peptide antigen MOG35-55 to induce experimental autoimmune encephalomyelitis (EAE), the mouse model for the human autoimmune disease multiple sclerosis. The mice were treated with acids at the early stages of the disease or before the onset of disease to determine the therapeutic and preventive effects, respectively.

### Therapeutic Effect—Acid Treatments Attenuated EAE Progression

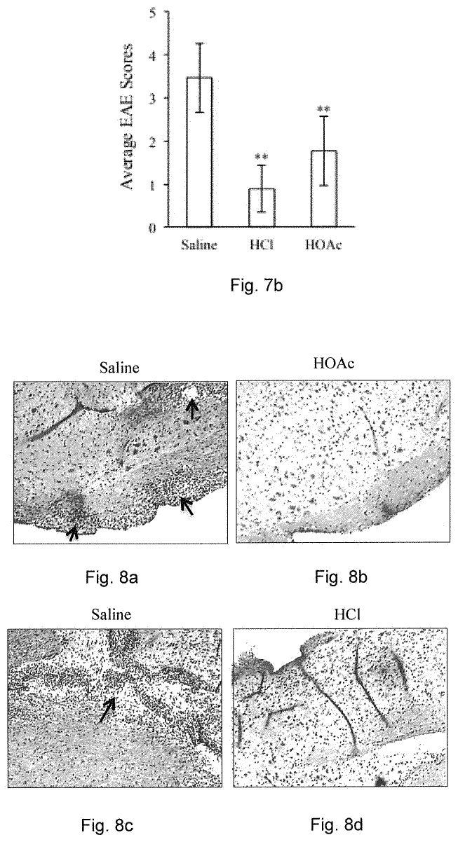

Mice at the early stages of EAE with the clinical scores of 1.5 to 2.5 received i.p. treatments with saline or saline plus HCl or HOAc as described in Materials and Methods. The mice were continuously monitored for disease progression for 6 or 7 days after the initiation of the treatments (some mice reached clinical score of 5 or higher after 6 days, therefore had to be sacrificed). Clinical scores of all control saline-treated mice continued to increase after the initiation of the treatment and peaked before day 4 after the initiation of the treatment, after which the disease entered the remission phase. (FIG. 7a). Within the observation period, 10 of the 11 these control mice reached clinical scores of 4 or above. (FIG. 7a). In contrast, none of the 5 mice treated with HCl progressed to clinical score of 4 or above. By day 4 after the initiation of treatment, all of the HCl-treated mice had clinical scores lower than their scores at the time of the initiation of treatment. The results of the HOAc-treated mice were similar to those of the HCl-treated mice. Only 1 of the 9 HOAc-treated mice reached clinical score of 4 or above. By day 4, the clinical scores of 6 of the mice were lower than their scores at the time of initial treatment, and 2 remained the same. (FIG. 7a). Prior to the remission at day 4, the average clinical score of the control mice was 3.46, whereas the average clinical score of the HCl-treated mice was 0.9, and that of the HOAc-treated mice was 1.78. (FIG. 7b). Histology analyses showed that on day 4 post initiation of treatment, the spinal cords of the control mice were infiltrated by inflammatory cells, whereas no obvious inflammation was observed in either the HCl- or HOAc-treated mice. (FIGS. 8a-8d).

### Preventive Effect—Acid Treatment Reduced and Delayed the Onset of EAE

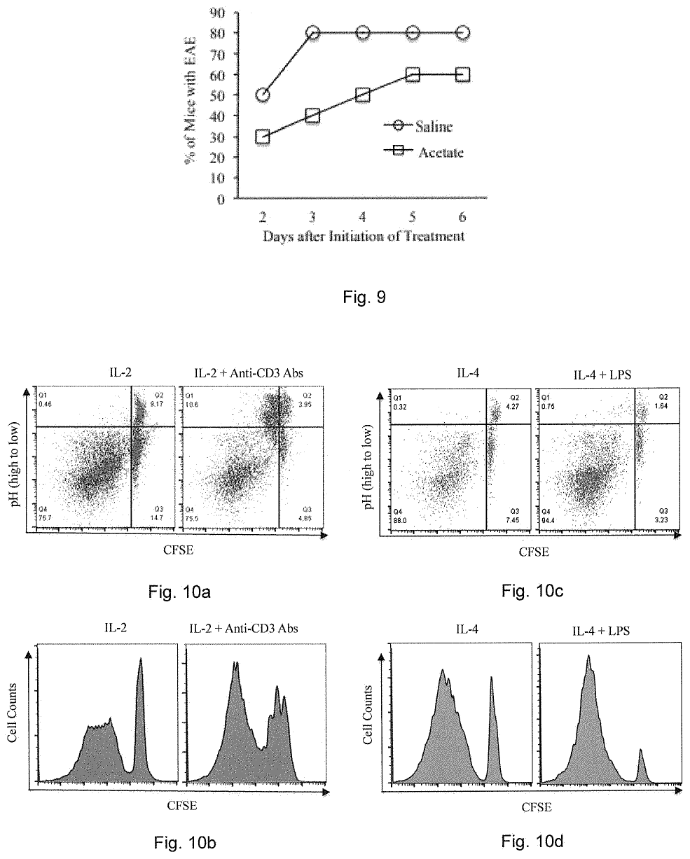

On day 11 after immunization with MOG35-55, at which time no clinical sign of EAE was observed, the mice received i.p. treatments with saline or saline plus HOAc. The preventive treatments were repeated every other day for two additional times. As shown in FIG. 9, clinical sign appeared 2 days after the initiation of treatments. In the control saline-treated mice, 50% of them developed EAE on day 2 after the initiation of treatment, the rate of disease onset quickly peaked at 80% on day 3. In the HOAc-treated mice, the rate of disease onset on day 2 after the initiation of treatment was 30%, and it did not reach the maximum rate of 60% until 5 days after the initiation of treatment.

### Example 3

### Control of Lymphocyte Populations and Proliferation in Draining Lymph Nodes by pH Modifiers

In the studies with animal disease models, thymic atrophy in mice treated with acids was noted. The thymus is one of few organs in adult animals that maintain active cell proliferation. Since lymphocyte proliferation in response to antigen stimulation is a quintessential characteristic of immune response, this observation led to the investigation of whether pH modifiers may modulate immune responses in the draining lymph nodes (DLNs), the primary sites of immune response to local antigen exposure, by controlling the lymphocyte populations and their proliferation. Further, lymph nodes are also some of the most common sites of tumor metastasis, and primary lymphomas can also arise in the lymph nodes. Thus, it was envisioned that pH modifiers could be used as alternatives to chemotherapy for cancer treatment. Here, the OVA sensitization and challenge model was used to provide an example that pH modifiers can in fact modulate cell population and proliferation in the lymph nodes.

### Positive Relationship Between pH Value and Lymphocyte Proliferation

To determine whether there is a natural relationship between peripheral lymphocyte proliferation and pH value, peripheral lymphocytes were labeled with the fluorescence dye CFSE. For the study of T cell proliferation, the labeled lymphocytes were cultured with either IL-2 alone or IL-2 and anti-CD3 antibodies. For the study of B cell proliferation, the labeled cells were cultured with either IL-4 or IL-4 and the B cell mitogen lipopolysaccharide (LPS). Cell proliferation was measured by serial dilution of the CFSE signals after each cell division, whereas the intracellular pH was measured by the pHrodo™ Red AM pH indicator whose fluorescence intensity inversely correlates with pH value. Proliferating cells with diluted CFSE signals were detected even in cultures with IL-2 or IL-4 alone in the total lymphocytes due to the presence of pre-existent proliferating lymphocytes. (FIGS. 10a-10d). As expected, proliferating cells (CFSE^{lo}) increased with concurrent decrease of non-proliferating cells (CFSE^{hi}) after stimulation by anti-CD3 antibodies or LPS. Most importantly, the highly proliferating cells that had undergone more than 3 divisions were almost exclusively in the cell populations of high intracellular pH, whereas cells that had fewer than 3 divisions were in populations of low intracellular pH. (FIGS. 10a-10d). Thus, this experiment has for the first time revealed a positive correlation between lymphocyte proliferation and their intracellular pH values.

### Control of the Total Number of Mediastinal Lymph Node Cells by pH Modifiers

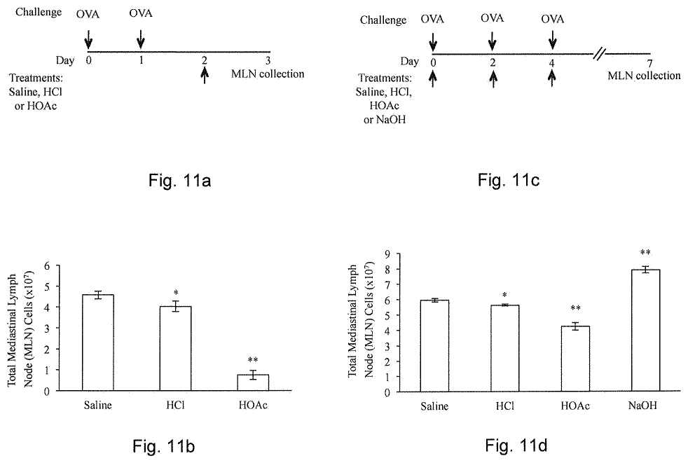

The DLNs are the primary location where active immune responses to local antigen exposure take place. The DLNs of the lungs are the mediastinal lymph nodes (MLNs). Two sets of experiments using the OVA sensitization and challenge models were carried out to investigate whether pH modifiers can affect lymphocyte populations and proliferation. In the first set of experiments, MLNs were collected from the mice 3 days after the first OVA challenge and 1 day after a single treatment. In the second set of experiments, MLNs were collected 7 days after the initial challenge and 3 days after the third treatments. (FIGS. 11a and 11c).

In the first set of experiments, the average numbers of total MLN were greatly reduced in HOAc-treated mice as compared with those of the saline-treated mice (0.75×10⁷ vs. 4.5×10⁷, or 84% reduction) 1 day after the treatment. (FIG. 11b). In the second set of experiments, the reduction of total MLN cells in the HOAc-treated mice was also dramatic 3 days after the final treatment, but less severe (4.23×10⁷ vs. 5.96×10⁷, or 29% reduction), which indicated on-going replenishment of the MLNs with new lymphocytes after the HOAc treatment. (FIG. 11d). The reduction of the total MLN cells in the HCl-treated mice was much less dramatic in either the first (4.0×10⁷, or 12% reduction) or second (5.6×10⁷, or 5.5% reduction) set of experiments, but nonetheless significant. (FIGS. 11b and 11d). In contrast, treatment with NaOH increased the total MLN cells (7.9×10⁷, or 32% increase). (FIG. 11d).

### Control of Different Lymphocyte Populations in MLNs by pH Modifiers

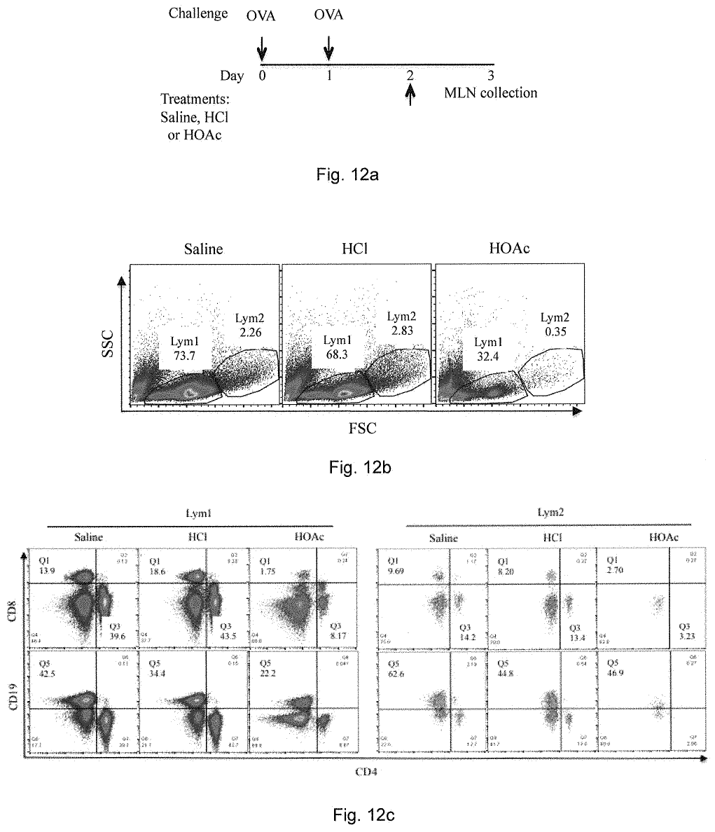

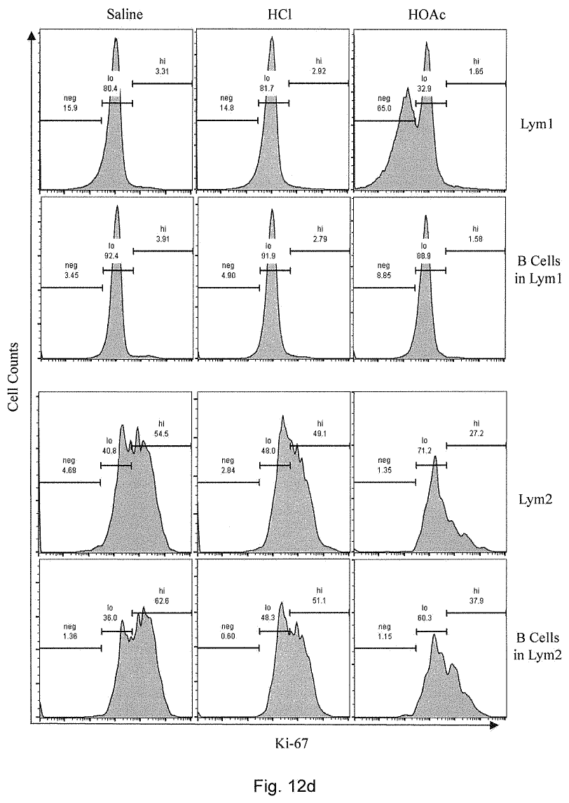

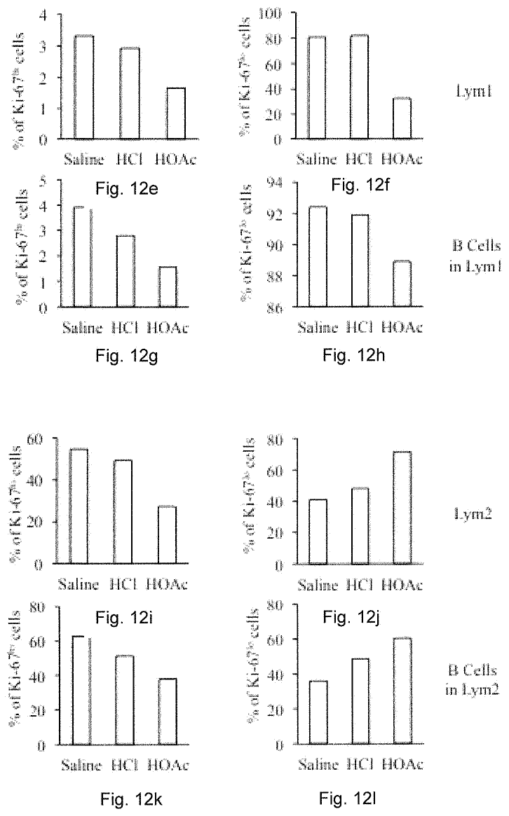

The total lymphocyte populations and the CD4, CD8 T cells and B cells within the lymphocyte populations were further analyzed. In the first set of experiments, MLNs were harvested 1 day after treatments and 3 days after the first OVA challenge. (FIGS. 11a, 12a). The majority of total lymphocytes in MLNs had relatively small sizes and low granularities and were termed as Lym1 populations. In addition, some lymphocytes were lymphoblasts with large sizes and high granularities, and they were termed as Lym2 populations. (FIG. 12b).

The percentages of both the Lym1 and Lym2 lymphocytes were greatly reduced in the HOAc-treated mice as compared with the saline-treated mice (32.4% vs. 73.7% and 0.35% vs. 2.26% for Lym1 and Lym2, respectively). The Lym1 population was also lower in HCl-treated mice (68.3%), but the Lym2 population was similar (2.83%). (FIG. 12b). Within the Lym1, the percentages of CD4, CD8 T cells and B cells in the HOAc-treated mice were all lower than those of the saline-treated mice (8.17% vs. 39.6%, 1.75% vs. 13.9%, and 22.2% vs. 42.5%, respectively), and similar reductions were also observed in the Lym2 (3.23% vs. 14.2%, 2.7% vs. 9.69%, and 46.9% vs. 62.6%). (FIG. 12c). To lesser degrees, the percentages of CD4, CD8 T cells and B cells in Lym2 of the HCl-treated mice were also reduced (13.4%, 8.2% and 44.8%). In contrast, within Lym1 population only the percentage of B cells of HCl-treated mice was reduced (34.4% vs. 42.5%). (FIG. 12c).

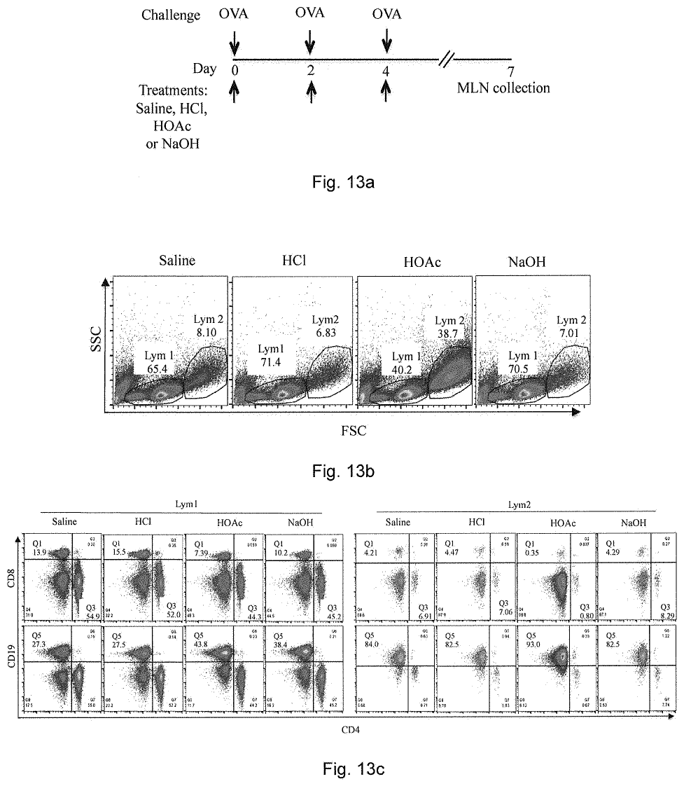

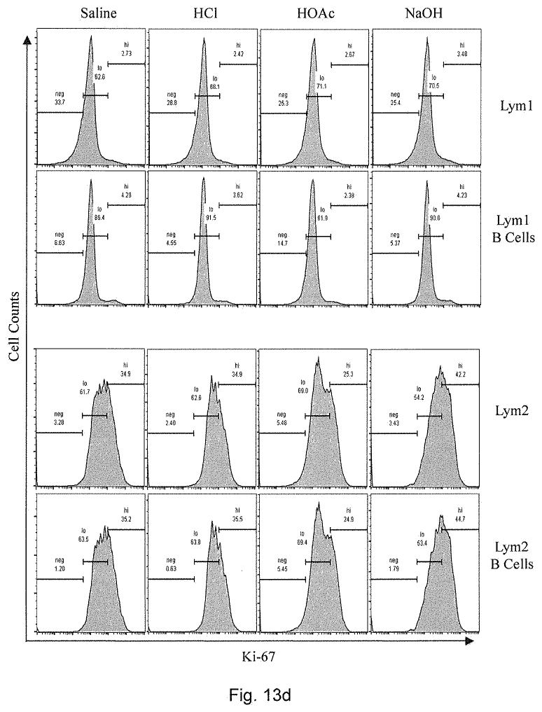

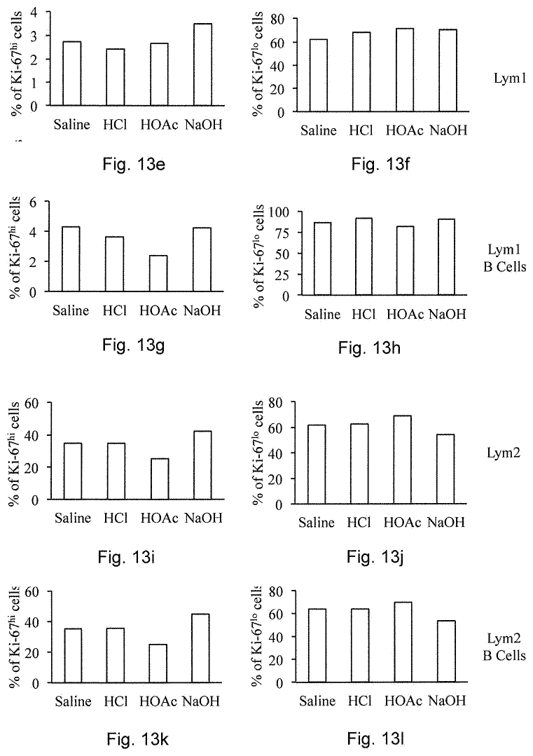

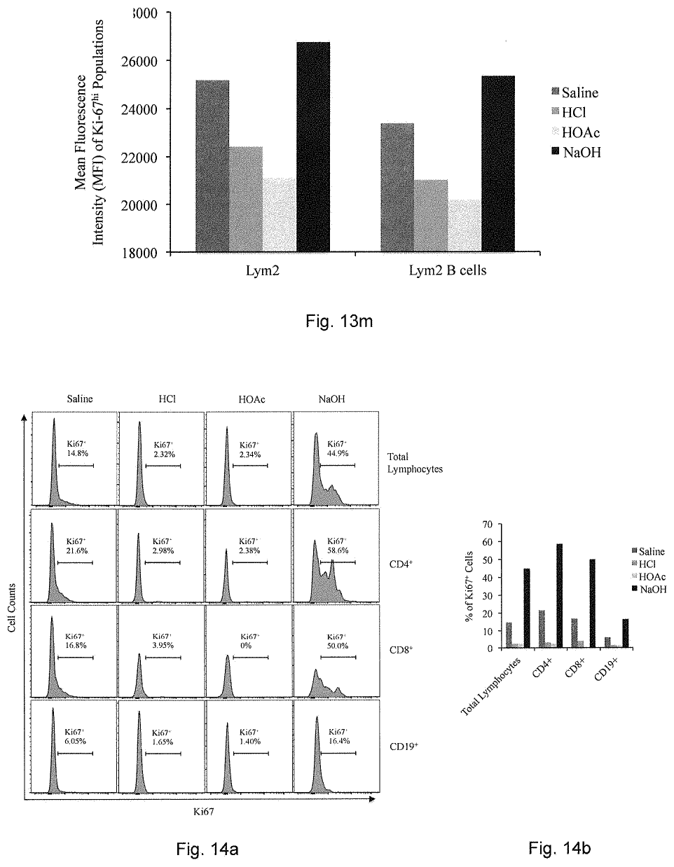

In the second set of experiments, MLNs were harvested 3 days after the final treatments and 7 days after the first OVA challenge. (FIGS. 11c, 13a). After such longer time of challenge and treatments, the Lym2 lymphoblast populations had expanded as compared with those of the first set of experiments. (FIG. 13b). While the combined percentages of lymphocytes (Lym1+Lym2) were similar in all treatment groups, the percentage of the Lym2, which comprised mostly B lymphoblasts, in the HOAc-treated mice was greatly increased in comparison with that of the saline-treated mice. (38.7% vs. 8.10%). (FIG. 13b).

Within the Lym1 lymphocytes, the percentages of CD4 and CD8 T cells in the HOAc-treated mice were lower than those of the saline-treated mice (44.3% vs. 54.9% or 19.3% reduction, and 7.39% vs. 13.9% or 46.8% reduction), whereas the percentage of B cells were higher (43.8% vs. 27.3% or 60.4% increase). Similar patterns were observed in the NaOH-treated mice, albeit to lesser degrees. In contrast, the percentages of CD4, CD8 T cells and B cells in Lym1 of HCl-treated mice were similar to those of the saline-treated mice. (FIG. 13c). Within the Lym2 populations, the reductions of the percentages of CD4 and CD8 T cells in the HOAc-treated mice were even greater than in Lym1 (0.8% vs. 6.91% or 88% reduction, and 0.35% vs. 4.21% or 91.7% reduction), whereas the percentage of B cells also proportionally increased (93% vs. 84%). In contrast, the percentages of CD4, CD8 T cells and B cells in Lym2 of the HCl- and NaOH-treated mice were similar to those of the saline-treated mice. (FIG. 13c).

In summary, acid treatments reduced, whereas alkaline treatment increased, the number of MLN cells during active immune responses. Within the lymphocytes, T cells were more susceptible than B cells to depletion by acid treatments. The MLNs appeared to be replenished by lymphocytes at later time after the acid treatments.

### Control of Cell Proliferation in MLNs by pH Modifiers

The proliferative statuses of the lymphocytes in the MLNs were determined by their expression of Ki-67. Ki-67 is a widely used, dependable marker for proliferating cells; its level of expression positively correlates with rRNA and DNA synthesis. (103). In the first set of experiments, i.e., at an early time after treatments and first challenge, the majority of the Lym1 cells expressed only low levels of Ki-67 but small percentages of the Lym1 cells expressed high levels of Ki-67. Nonetheless, in the HOAc-treated mice, the percentage of Ki-67^{hi} Lym1 cells was less than half of that of the saline-treated mice (1.65% vs. 3.31%), the percentage of the Ki-67^{hi} Lym1 cells in the HCl-treated mice (2.92%) was also reduced but to a lesser degree. In the HOAc-treated mice, even the percentage of Ki-67^{lo} cells in Lym1 was also reduced (32.9% vs. 80.4%), whereas the percentage of Ki-67^{lo} Lym1 cells in HCl-treated mice was similar to that of the saline-treated mice (FIGS. 12d-12f). The Lym2 cells were overall much more proliferative than the Lym1 cells. In the HOAc-treated mice the percentage of Ki-67^{hi} Lym2 cells was again less than half of that of the saline-treated mice (27.2% vs. 54.5%), the percentage of Ki-67^{hi} Lym2 cells in HCl-treated mice (49.1%) was also reduced, albeit less dramatically. (FIGS. 12d and 12i). Thus, proliferating lymphocytes in the MLNs were preferentially depleted by acid treatments. Lymphocytes with high proliferative profiles were more susceptible to the depletion than those with low proliferative profiles.

As described in the previous section, B cells in MLNs were less susceptible to depletion by acid treatments. Therefore, it is necessary to determine whether proliferative status of B cells also correlates with their susceptibility to depletion by acid treatments. Indeed, in both the Lym1 and Lym2 lymphocyte populations, the percentages of Ki-67^{hi} B cells were the lowest in HOAc-treated mice (1.58% and 37.9% in Lym1 and Lym2, respectively), followed by those in HCl-treated mice (2.79% and 51.1%), and highest in the saline-treated mice (3.91% and 62.6%). (FIGS. 12d, 12g, 12k).

In the second set of experiments, i.e., at a later time after treatments and first challenge, the percentages of Ki-67^{hi} Lym1 cells were slightly lower in HCl- or HOAc-treated mice (2.42% and 2.67%, respectively), but higher in NaOH-treated mice (3.48%), than that of saline-treated mice (2.73%). (FIGS. 13d and 13e). The percentages of Ki-67^{hi} Lym1 B cells were also lower in HCl- or HOAc-treated mice (3.62% and 2.38%) than, but similar in NaOH-treated mice (4.23%) to, that of saline-treated mice (4.29%). (FIGS. 13d and 13g). The percentage of Ki-67^{hi} Lym2 cells in HOAc-treated mice (25.3%) was lower, that of NaOH-treated mice (42.2%) was higher, than that of saline-treated mice (34.9%). (FIGS. 13d and 13i). The percentages of Ki-67^{hi} Lym2 B cells followed the same pattern, with those of saline-, HOAc- and NaOH-treated mice being 35.2%, 24.9% and 44.7%, respectively. (FIGS. 13d and 13k). In contrast, the percentages of Ki-67^{hi} Lym2 and Lym2 B cells in HCl-treated mice (34.9% and 35.5%) were essentially the same as those of the saline-treated mice. (FIGS. 13d and 13k). However, the Ki-67 mean fluorescence intensities of the Ki-67^{hi} Lym2 and Lym2 B cells of the HCl-treated mice, like those of the HOAc-treated mice, were much lower, whereas they were higher in NaOH-treated mice, than those of the saline-treated mice. (FIG. 13m).

Thus, acid treatments not only preferentially depleted Ki-67^{hi} cells but also lower their proliferative profiles, whereas alkaline treatment had the opposite effects.

### Control of Lymphocyte Proliferation by pH Modifiers In Vitro

The fluidity of the in vivo environment could mask the full effects of the pH modifiers. For example, circulating and/or newly activated lymphocytes could replenish the MLNs after lymphocyte depletion by acid treatments, therefore analysis of ex vivo MLNs could have underestimated the effects of the acid treatments. Further, the kinetics of absorption and the subsequent transportation of the pH modifiers to the MLNs, as well as the excretion of the pH modifiers from MLNs and other organs or tissues, are unknown. As such, the actual time and concentration at which the pH modifiers acted in the MLNs could not be determined. These complexities are particularly relevant to the observation that i.t. instillation of HCl was less effective than i.t. instillation of HOAc on the MLN cells. However, it cannot be determined whether this is due to intrinsic difference between HCl and HOAc or their different rates of transportation to the MLNs hence their different local concentrations in the MLNs.

Thus, to assess the full potentials of the pH modifiers, in vitro experiments were carried out, in which primary lymph node cells were treated with saline or saline plus the pH modifiers in 100% FBS in 37° C. water bath. Such in vitro culture systems were designed to be free of interference by pH buffering agents in common tissue culture system. The final concentration of the pH modifiers in the in vitro cultures was set to be 8.75 mM. Assuming that the total volume of the blood of a mouse is about 2 ml (104, 105), this concentration would approximate the whole-body blood concentration of the pH modifier that could be achieved by injecting 200 μl of saline plus 87.5 mM pH modifier used in the in vivo i.p. treatments. As shown in FIGS. 14a and 14b, in vitro treatments with HCl or HOAc almost eliminated all Ki-67⁺ (Ki-67^{hi and lo}) cells in the live total lymphocytes, CD4, CD8 T cells and B cells. In contrast, NaOH treatment more than tripled the percentages of Ki-67⁺ cells in total lymphocytes and CD8 T cells, and nearly tripled in the CD4 T cells and B cells.

Thus, the in vitro results collaborated the results of the in vivo treatments that acid treatments preferentially depleted, whereas alkaline treatments increased, the proliferating lymphocytes in the MLNs. Not only highly proliferating cells can be depleted by acid treatments, cells of low proliferative profiles can also be depleted by increasing the concentration of the pH modifiers and/or the duration of treatments. The stronger effects in the in vitro than in vivo experiments showed that the effects of pH modifiers are determined not only by their chemical properties but also their dose (concentration) and frequency (duration) of treatment.

### Example 4

### Control of Tumor Cell Population and Proliferation by pH Modifiers

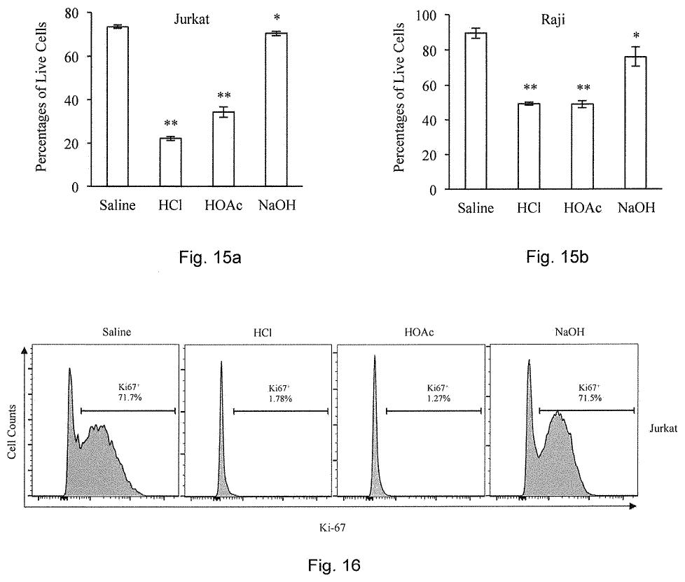

To determine whether the pH modifiers have similar effects on proliferating tumor cells, two human tumor cell lines the T cell leukemia cell line Jurkat and the B cell leukemia cell line Raji were studied. Jurkat and Raji cells were treated with saline or saline plus HCl, HOAc, or NaOH in FBS. Compared with saline treatment, HCl and HOAc treatments greatly reduced the viabilities of the Jurkat (22.1% and 34.3% vs. 73.4%) and Raji (49.2% and 48.9% vs. 89.4%) cells. In contrast, NaOH treatment only slightly decreased the viabilities of Jurkat and Raji cells (70.4% and 76.2%, respectively). (FIGS. 15a and 15b).

### Acid but not Alkaline Treatments Preferentially Depleted Proliferating Tumor Cells

While tumorigenesis is a complex biological process, the primary feature of tumor cells is dysregulated cell proliferation. Thus, the correlation between tumor proliferation and their susceptibility to depletion by pH modifiers was investigated using the Jurkat cells as a model system. After saline treatments, the majorities of Jurkat maintained Ki-67 expression (71.7%). However, few, if any, (<2%) Jurkat cells that survived the HCl or HOAc treatments expressed Ki-67. In contrast, the percentage of Ki-67⁺ Jurkat cells after NaOH treatment was similar to those after saline treatment. (FIG. 16).

It must be pointed out that the finding disclosed herein that alkaline treatment maintains or promotes normal and neoplastic cell proliferation is contrary to the belief that the so-called “alkaline therapy”, i.e., consuming certain “alkaline” foods and beverages, suppresses tumor genesis/growth, which despite promotion by the media and salespersons has been lacking scientific evidence. (88).

### Example 5

### Depletion of Proliferating Cells in the Bone Marrow by pH Modifiers

Like lymph nodes, bone marrow is one of the most common sites for tumor metastasis. (106). In addition, primary tumors or tumor-like lesions can also occur in the bone. (107). This Example 5 will demonstrate that treatments with pH modifiers can effectively deplete or inhibit the proliferation of proliferating cells of different cell lineages in the bone marrow, therefore they can be used to deplete tumor cells in the bone marrow. In this example, C57BL/6 mice were treated with i.p. injection of saline, or saline plus HCl or HOAc every other day for 3 times. Bone marrow cells were extracted from both tibias 1 day after the third treatment for the study of B lineage cells, and for the studies of other cell types 3 days after the third i.p. treatment with saline or saline plus HOAc, and alternatively, mice received one i.t. injection of saline or saline plus HOAc, bone marrow was extracted from tibias the next day.

### Depletion of Proliferating B Lineage Cells in Bone Marrow by pH Modifiers

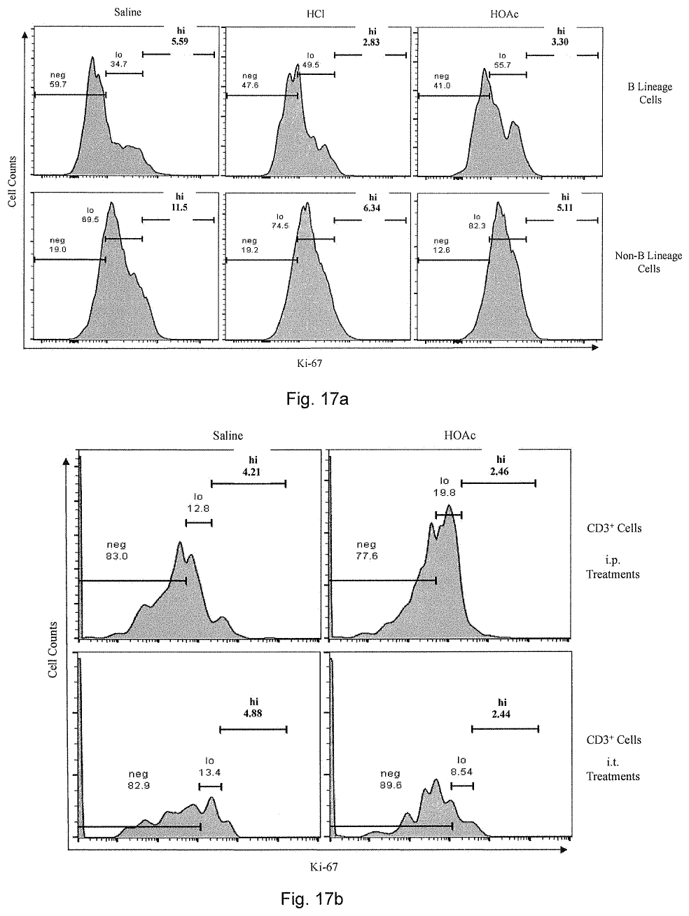

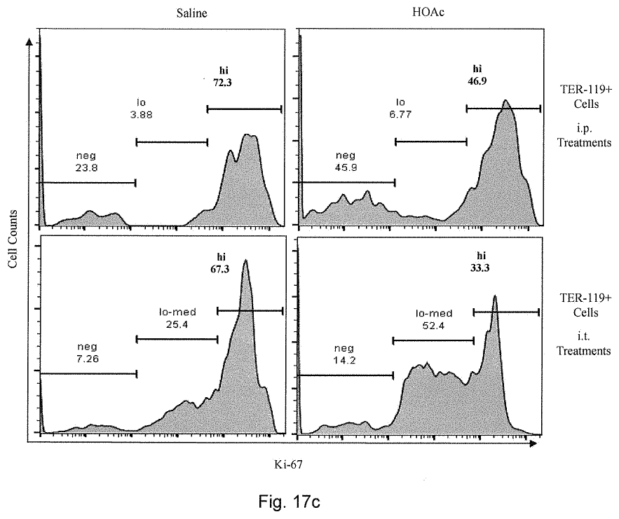

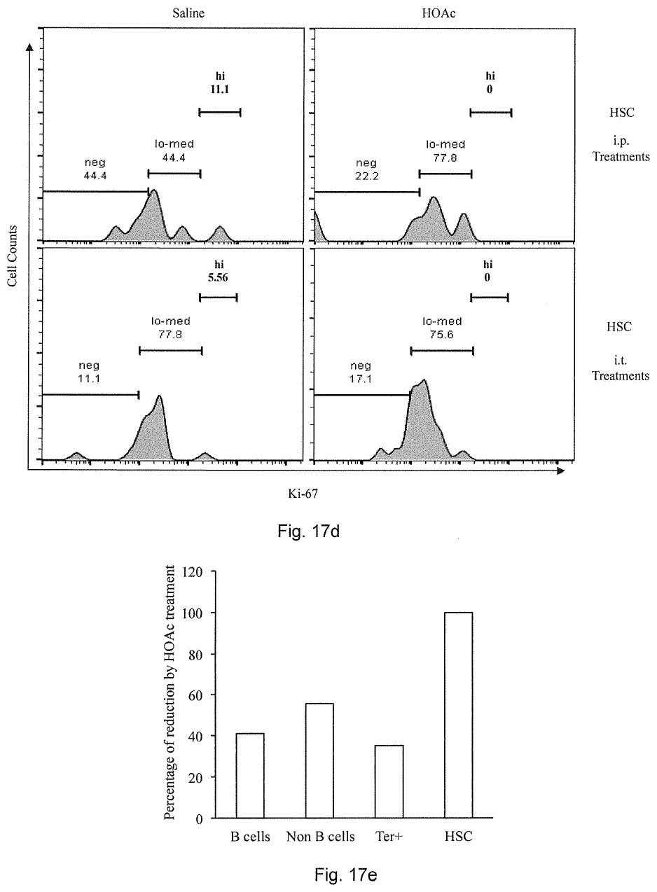

The first cell type analyzed was the B (cell) lineage. Bone marrow is not only the primary organ for B cell lymphopoiesis, circulating normal or neoplastic B cells also home to the bone marrow. The non-granulocyte white bone marrow (WBM) cells comprise B cells at all maturation stages, all of which express the surface marker CD19. The B lineage (CD19⁺) cells in the bone marrow comprise distinct populations with high, low or no expression of Ki-67. In mice that had received i.p. treatment with saline, 5.59% of bone marrow B lineage cells were Ki-67^{hi}. This population was decreased to 2.83% and 3.3% by i.p. treatments with HCl and HOAc, respectively. (FIG. 17a). Similarly, in saline-treated mice, 11.5% of the non-B lineage cells of the non-granulocyte WBM cells were Ki-67^{hi}, this population was decreased to 6.34% and 5.11% by i.p. treatments with HCl and HOAc, respectively.

### Depletion of Proliferating T Cells in Bone Marrow by pH Modifiers

In mice i.p. treated with saline, 4.21% T (CD3⁺) cells in the bone marrow were Ki-67^{hi}. This population of the T cells decreased to 2.46% in mice i.p. treated with HOAc. (FIG. 17b, upper panels). In addition to i.p. treatments, T cells from mice that received i.t. treatment were also studied. The percentage of Ki-67^{hi} T cells decreased to 2.44% in mice i.t. treated with HOAc from 4.88% in mice i.t. treated with saline. (FIG. 17b, lower panels).

### Depletion of Proliferating Erythroid Lineage Cells in Bone Marrow by pH Modifiers

In mice i.p. treated with saline, 72.3% erythroid (TER-119⁺) lineage cells were Ki-67^{hi}, which was decreased to 46.9% in mice i.p. treated with HOAc. Likewise, Ki-67^{hi} erythroid lineage cells were decreased to 33.3% in mice i.t. treated with HOAc from 67.3% in mice i.t. treated with saline. (FIG. 17c).

### Depletion of Proliferating HSC in Bone Marrow by pH Modifiers

Hematopoietic stem cells (HSC) in the bone marrow are defined as lineage negative (Lin⁻) (stained negative for B220, CD3, CD11 b, CD11c, CD48, Ly6G, TER-119) Sca-1⁺ c-kit⁺ CD150⁺ WBM cells. (108). In mice i.p. treated with saline, 11.1% the HSC were Ki-67^{hi}, however, in mice i.p. treated with HOAc, no Ki-67^{hi} HSC were detected. (FIG. 17d, upper panels). Similarly, in mice i.t. treated with saline, 5.56% HSC were Ki-67^{hi}, whereas in mice i.t. treated with HOAc, no Ki-67^{hi} HSC were detected. (FIG. 17d, lower panels).

### Comparison of Susceptibility to Acid-Induced Depletion Among Cells of the Bone Marrow

FIG. 17e summarizes the reductions of Ki-67^{hi} populations of the different lineages in the WBM cells by the i.p. treatment with HOAc. The percentage of reduction is calculated as: 100×((% of saline-treated mice−% of HOAc-treated mice)/% of saline-treated mice). Ki-67^{hi} B cells, non-B lineage cells, Ter-119⁺ cells and HSC are compared. The Ki-67^{hi} cells in HSC were the most susceptible and were essentially eliminated by the HOAc treatment, followed by the overall non-B cells, B cells and Ter-119⁺ cells.

### Example 6

### Control of Cell Proliferation in Lung Tissue by pH Modifiers

The lung is another common site for tumor metastasis and primary lung cancers. (109, 110). In addition, inflammatory cell proliferation and hyperplasia of lung structural functional cells such as pneumocytes, fibroblasts and goblet cells are characteristics of pulmonary inflammation caused by infections, allergen exposures or other stimulants. (6, 7). For these reasons, it was envisioned that pH modifiers can be used to treat clinical conditions associated with cell proliferation in the lungs. This example uses the asthma model to show the effects of pH modifiers on proliferating cells in the lung tissue.

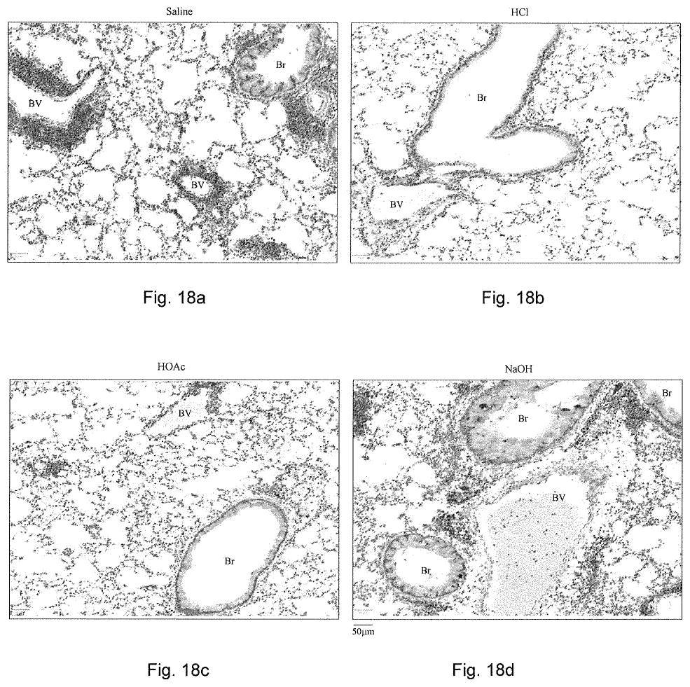

Mice sensitized and challenged with OVA received i.t. treatments with saline or saline plus HCl or HOAc, or i.p. treatments with saline plus NaOH as in the asthma model. Lung tissue sections of the mice were analyzed for the expression of Ki-67 by immunohistochemistry. In the lungs of the saline-treated mice, Ki-67⁺ cells were abundant in the infiltrating inflammatory cells. In addition, the alveolar walls predominantly comprised multiple layers of pneumocytes, indicative of pneumocyte hyperplasia. Consistent with this observation, abundant Ki-67⁺ cells were found in the alveolar walls, as well as in the bronchiolar walls. (FIG. 18a).

In the lungs of mice treated with HOAc or HCl, inflammatory cells were greatly reduced. In the remaining residual inflammatory cells, there were much fewer Ki-67⁺ cells than in the inflammatory cells of the lungs of the saline-treated mice. The alveolar walls of these mice predominantly comprised a single layer of pneumocytes. Ki-67⁺ cells were only sporadically detected in the lung tissues. (FIGS. 18b and 18c). In the lungs of the NaOH-treated mice, although there were fewer inflammatory cells in the perivascular area, Ki-67⁺ positive cells in the remaining inflammatory cells were abundant; the Ki-67⁺ cells were also abundant among the inflammatory cells in the peribronchial areas. Like in the lungs of the saline-treated mice, alveolar walls of the NaOH-treated mice predominantly comprised multiple layers of pneumocytes, among which Ki-67⁺ cells were abundant. Ki-67⁺ positive cells were also abundantly detected in the leukocytes inside the blood vessels in the lungs of the NaOH-treated mice. (FIG. 18d). Further, the Ki-67 staining in the NaOH-treated lungs was stronger than that in the saline- or acid-treated mice. (FIGS. 18a-18d).

Thus, acid treatments can effectively deplete proliferating cells of different cell types in the lungs, whereas NaOH treatment enhances cell proliferation.

### Example 7

### Control of Proliferating Cells in the Intestines by pH Modifiers

Like the bone marrow and thymus, intestines are another organ that maintain robust cell proliferation in adults. Thus, it is not surprising that colorectal cancer is the fourth most common cancer, and intestinal cancers can also originate in the small intestines. (111, 112). In addition, intestinal epithelial cell renewal/proliferation is necessary for maintaining intestinal villi. Therefore, it was envisioned that pH modifiers could be used to control cell proliferation in the gastrointestinal (GI) tracks hence to treat cancers in the GI tracks and to manage body weight for the treatment and prevention of obesity. This example shows a model of controlling proliferating cells in the intestines by pH modifiers.

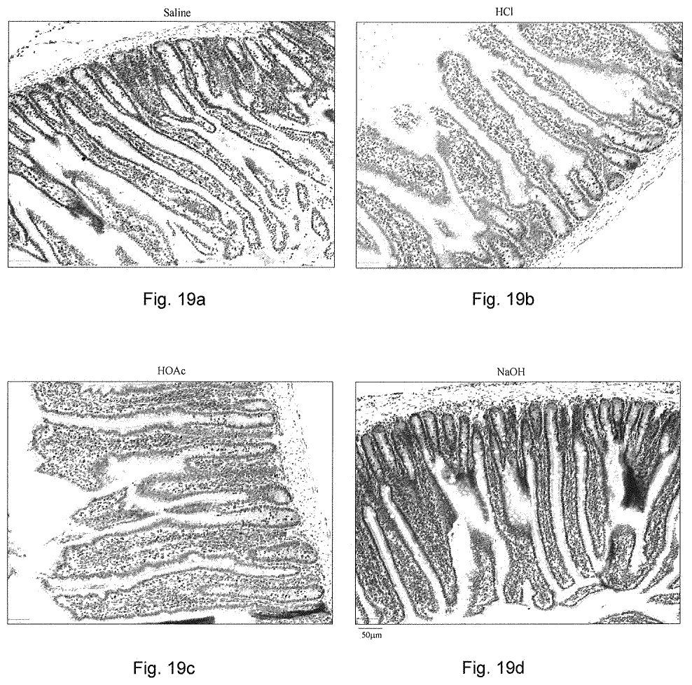

Mice were i.p. treated with saline or saline plus HCl, HOAc or NaOH every other day for a total of 3 doses. One day after the third dose, mice were sacrificed, and tissue sections of the intestines were analyzed by immunohistochemistry staining of Ki-67 with hematoxylin counter staining. As shown in FIGS. 19a-19d (treatment with saline, HCl, HOAc, and NaOH, respectively), there were more Ki-67⁺ cells in the intestinal villi of saline- or NaOH-treated mice than the HCl- or HOAc-treated mice. In addition, the Ki-67 staining was the strongest for the NaOH-treated mice, followed by that of the saline-treated mice, which was in turn stronger than those of the HCl- or HOAc-treated mice.

Thus, like in the lungs, acid treatments can deplete proliferating cells and/or inhibit their proliferation, whereas alkaline treatment enhances cell proliferation in the intestines.

### Example 8

### Control of Lymphopoiesis in the Thymus by pH Modifiers

### Acid but not Alkaline Treatment Caused Thymic Atrophy

As alluded in Example 3, thymic atrophy was observed in mice of various disease models used in this invention, such as the asthma models. Since thymus is one of the few organs in adults that maintain active cell proliferation, this observation was the initial clue leading to the studies throughout this invention to determine that cell proliferation is related to pH and can be modulated by pH modifiers. Consistent with thymus as the primary lymphoid organ for T cell lymphopoiesis, Example 8 will show that by their abilities to control cell proliferation in the thymus pH modifiers can be used to control T cell lymphopoiesis.

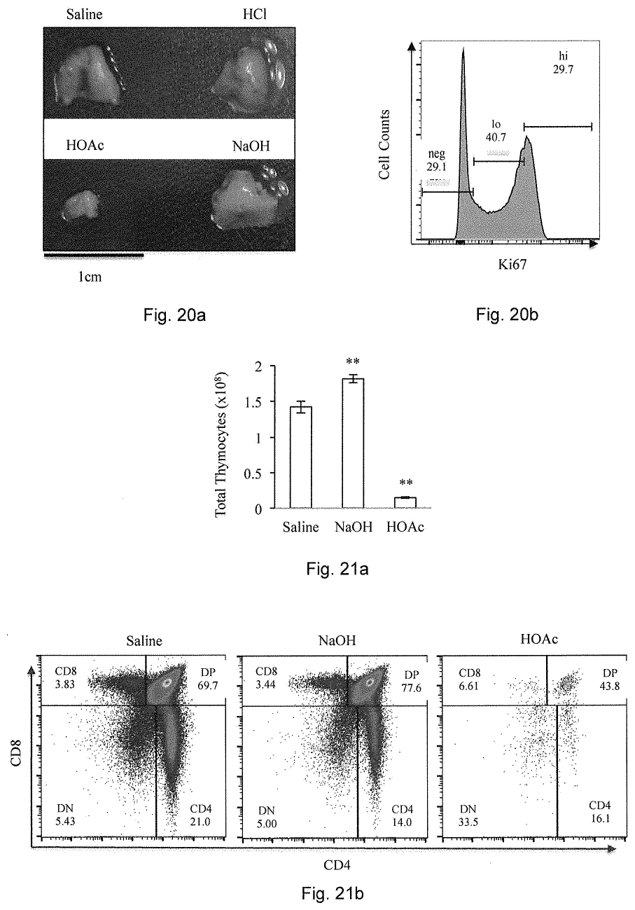

In the asthma model, OVA sensitized mice were challenged with OVA by i.t. injection every other day for 3 times. The mice received i.t. treatment with saline, HCl or HOAc at the same time as challenge, or i.p. treatment with NaOH 1 hour after the challenge. Thymuses were harvested 3 days after the final challenge and treatment. By gross examination, the sizes of the thymuses of the HCl- or HOAc-treated mice were dramatically smaller than, whereas thymic size of the NaOH-treated mice was similar to, that of the thymuses of the saline-treated mice. (FIG. 20a). As expected, the majority of thymocytes (>70%) are proliferating cells, consistent with the notion that thymus is an organ of active cell proliferation. The thymocytes can be divided into Ki-67 high, low and negative populations. (FIG. 20b).

### Effects of In Vivo Treatments with pH Modifiers on Thymocyte Cell Populations

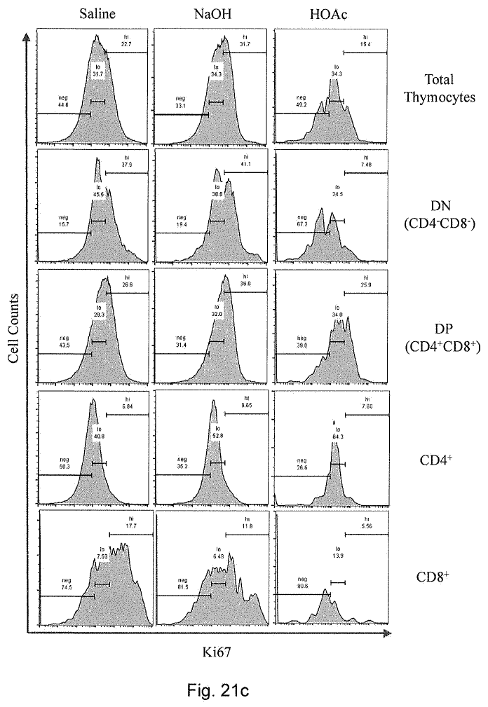

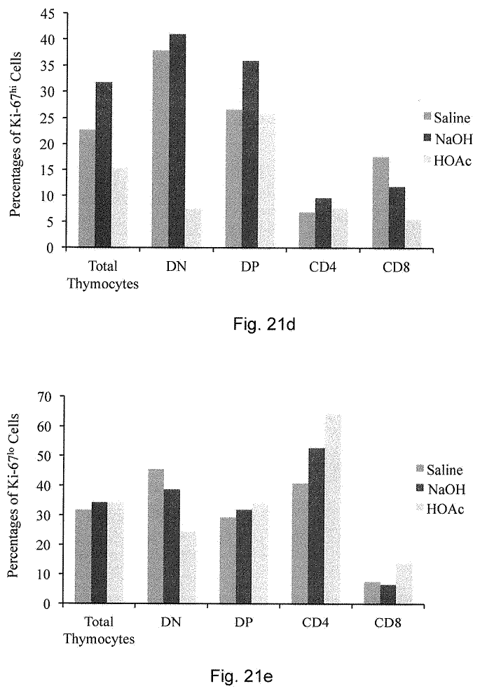

Thymuses from representative mice of the different treatment groups were further analyzed for the effects of the pH modifiers on T cell lymphopoiesis. The total numbers of thymocytes of NaOH-treated mice increased by about 28% as compared with those of the saline-treated mice despite less visible size differences by gross examination. In contrast, the total numbers of thymocytes of HOAc-treated mice was decreased by almost 90%. (FIG. 21a). While the thymocytes of all treatment groups comprised the typical four subpopulations defined by the expression of CD4 and CD8, relative proportions of the subpopulations varied considerably among the groups. Most notably, the percentage of the highly proliferative DP (CD4⁺CD8⁺) subpopulation was higher in the NaOH-treated mice (77.6%) but lower in the HOAc-treated mice (43.8%) than that in the saline-treated mice (69.7%). (FIG. 21b). This result is consistent with the results in the previous examples that have shown that NaOH increased, but HOAc decreased, highly proliferative cells.

### Effects of In Vivo Treatments with pH Modifiers on the Proliferative Statuses of Thymocytes

Further analysis of Ki-67 expression showed that the percentage of highly proliferative (Ki-67^{hi}) total thymocytes was higher in the NaOH-treated (31.7%), but lower in HOAc-treated (15.4%), mice than that of the saline-treated mice (22.7%). This pattern was also observed in the highly proliferative DN and DP subpopulations. The relative proportions of the low proliferative (Ki-67^{lo}) thymocytes were generally opposite to those of their highly proliferative counterparts. (FIGS. 21c-21e).

### Effects of In Vitro Treatments with pH Modifiers on Thymocyte Proliferation

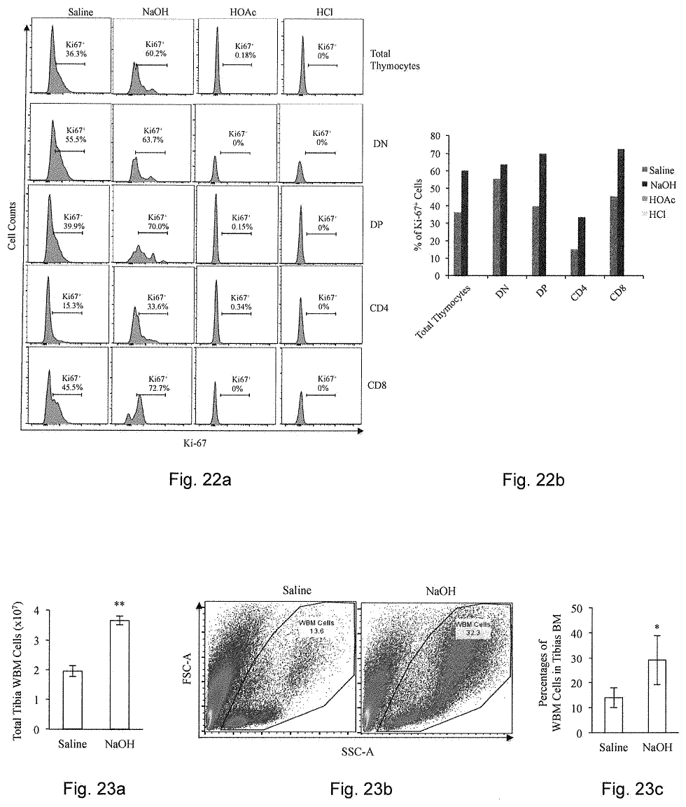

Like in the draining lymph nodes, fluidic in vivo environment in the thymus made it difficult to assess the full extent, to which the pH modifiers could affect thymocyte proliferation. To fully assess the potential effects of the pH modifiers on the thymocyte proliferation, in vitro treatments with pH modifiers were carried out, in which thymocytes were cultured in FBS comprising saline or saline plus the pH modifiers NaOH, HOAc or HCl. After the in vitro NaOH treatment, total thymocytes and all 4 subpopulations had higher percentages of Ki-67⁺ cells than their counterparts from the saline-treated cultures. In contrast, there were almost no Ki-67⁺ cells in thymocytes from cultures treated with HOAc or HCl. (FIGS. 22a and 22b).

In summary, Example 8 shows that treatments with pH modifiers such as NaOH that increase pH or maintain high pH enhance thymocyte proliferation or T cell lymphopoiesis. In contrast, treatments with pH modifiers such as HOAc that decrease pH decrease thymocyte proliferation and T cell lymphopoiesis. The differences between in vivo and in vitro treatments demonstrated that the effect of a pH modifier on the same thymocytes is determined not only by the chemical property of the pH modifier but also by the concentration/dose of the pH modifier and the duration of the treatment.

### Example 9

### Control of Hematopoiesis in the Bone Marrow by pH Modifiers

### Increase of Total White Bone Marrow (WBM) Cells by pH Modifiers

Based on data from the preceding examples, it was envisioned that alkaline treatment could enhance hematopoiesis in the bone marrow. To provide an example of this aspect of the present invention, C57BL/6 mice received i.p. treatments with saline or saline plus NaOH every other day for a total of 3 doses. Bone marrows were extracted from both tibias on day 1 or 3 after the final treatment. The average total number of WBM cells increased by 88% in the NaOH-treated mice as compared with that of the saline-treated mice. (FIG. 23a). Further, the average percentage of WBM cells in total bone marrow cells increased in the NaOH-treated mice to over 29% from less than 14% in the saline-treated mice. (FIGS. 23b and 23c).

### Increase of B Cell Lymphopoiesis by pH Modifiers

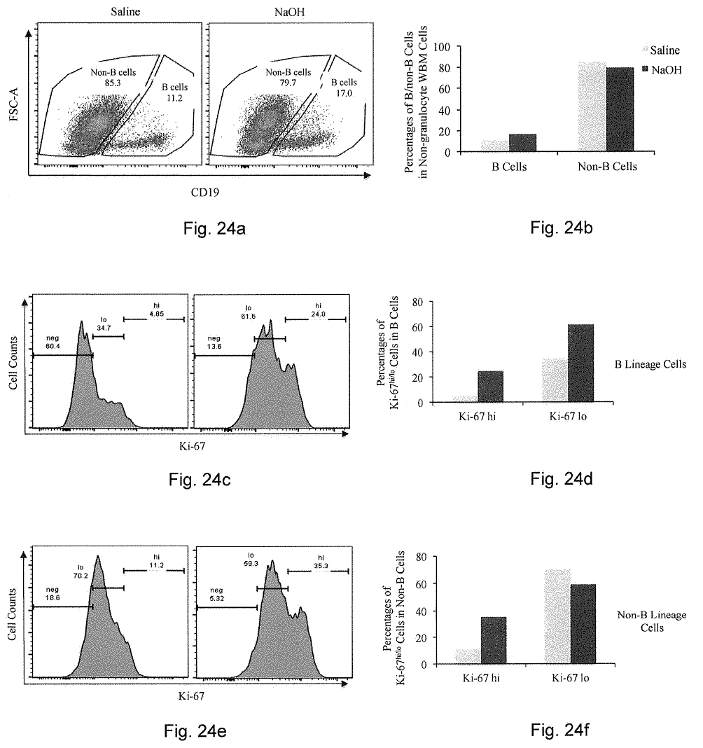

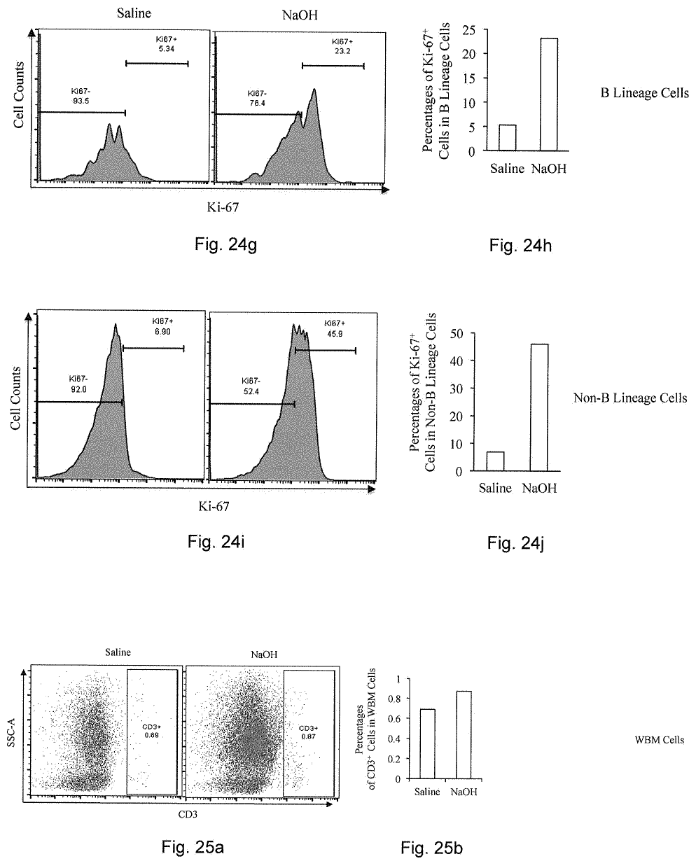

Bone marrow is the primary lymphoid organ for B cell lymphopoiesis in adults. B lineage cells in all developmental stages expressed the surface marker CD19. The percentage of CD19+ or B lineage cells in the non-granulocyte WBM cells was about 11% in saline-treated mice. One day after three i.p. treatments with NaOH, the B lineage cells increased to 17%. (FIGS. 24a and 24b). The percentages of both the Ki-67^{hi} and Ki-67^{lo} cells in the B lineage cells were much increased over those of the saline-treated mice (24.8% and 61.6% vs. 4.85% and 34.7%). (FIGS. 24c and 24d). The increase of the percentage of Ki-67^{hi} cells was also observed in the non-B lineage cells (35.3% vs. 11.2%). (FIGS. 24e and 24f). The increase of the percentages of proliferating B and non-B lineage cells lasted at least 3 days after the final NaOH treatment. (FIGS. 24g-24j). In the following paragraphs, other cell lineages were analyzed using bone marrows extracted on day 3 after the final treatments.

### Enhancement of T Cell Expansion in Bone Marrow by pH Modifiers

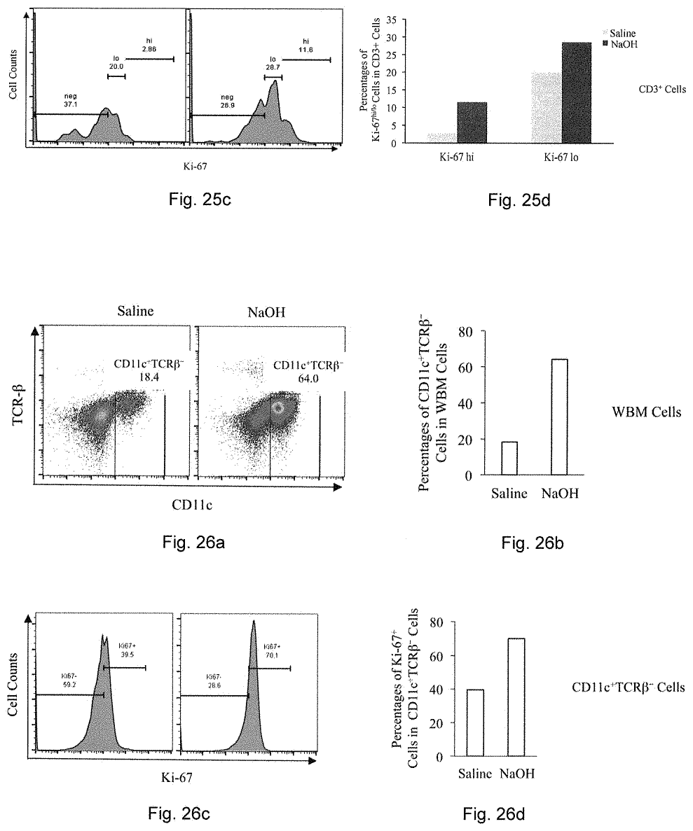

Although T (CD3⁺) cells are not generated in the bone marrow, they can migrate to the bone marrow through circulations. The percentage of T cells in the WBM cells slightly increased in the NaOH-treated mice to 0.87% from 0.69% in saline-treated mice. (FIGS. 25a and 25b). The percentages of both the Ki-67^{hi} and Ki-67^{lo} cells in the T cells also increased in the NaOH-treated mice (11.6% and 28.7% vs. 2.86% and 20%). (FIGS. 25c and 25d).

### Increase of CD11c⁺ Cell Genesis in Bone Marrow by pH Modifiers

CD11c⁺ cells are a major cell lineage in the bone marrow. CD11c is a marker for innate lymphoid cells such as dendritic cells, NK cells, NKT cells, etc., but may also be expressed on some activated T cells. (113). In the WBM cells of the NaOH-treated mice, the percentage of CD11c⁺ TCR⁻ innate lymphoid lineage cells was increased to 64% from 18.4% in the saline-treated mice. (FIGS. 26a and 26b). Although this cell population was overall not very proliferative as they expressed only low levels of Ki-67, the percentage of Ki-67⁺CD11c⁺ TCR⁻ cells in the NaOH-treated mice was much higher than in the saline-treated mice (70.1% vs. 39.5%). (FIGS. 26c and 26d).

### Increase of Ly6G⁺ Cell Genesis in Bone Marrow by pH Modifiers

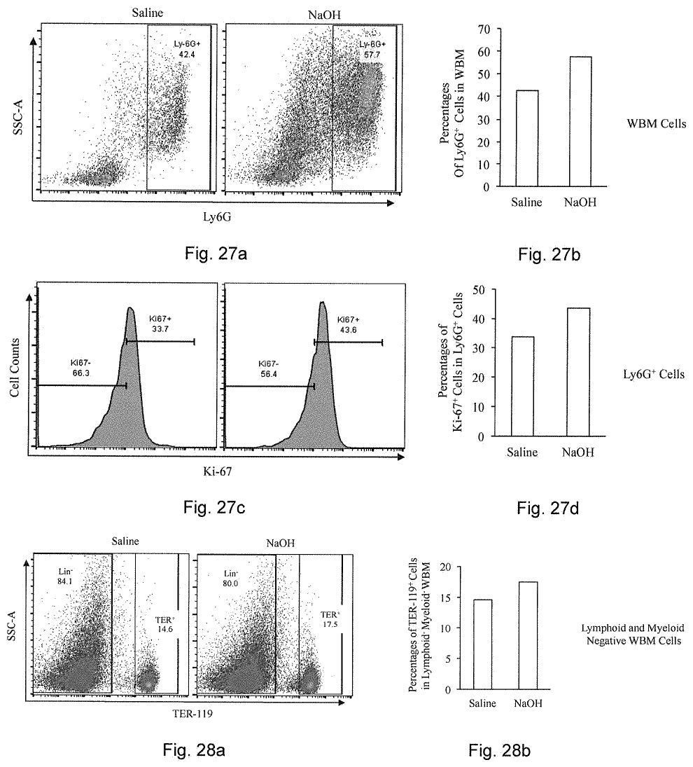

Ly6G is a marker for myeloid cells. Particularly, neutrophils express high levels of Ly6G. (101). To lesser extents, other granulocytes and bone marrow monocytes also express this marker. (114). (also see www.ebi.ac.uk/gxa/genes/ensmusg00000022582). In NaOH-treated mice, the percentage of Ly6G⁺ cells in the WBM cells was higher than that of saline-treated mice (57.7% vs. 42.4%), so was the percentage of Ki-67⁺ cells within the Ly6G⁺ population (43.6% vs. 33.7%). (FIGS. 27a-27d).

### Increase of TER-119⁺ Cell Genesis in Bone Marrow by pH Modifiers

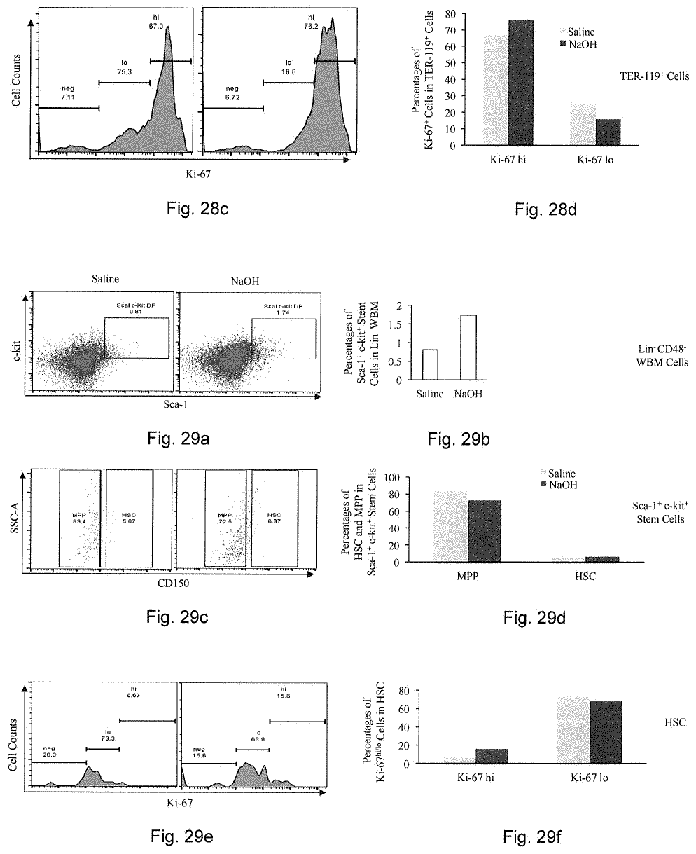

TER-119 is an erythroid cell specific surface marker, it is expressed on proerythroblasts and mature erythrocytes. (115). Since the lymphoid and myeloid cell lineages were increased in the WBM cells of NaOH-treated mice, to better assess the impact of NaOH treatment on the erythroid lineages, these cells were analyzed in the lymphoid and myeloid negative WBM cells (stained negative for B220, CD3, CD11b, CD11c, Ly6G and CD48). The percentage of TER-119⁺ cells in the NaOH-treated mice was slightly higher than that in the saline-treated mice in the lymphoid and myeloid negative WBM cells (17.5% vs. 14.6%). (FIGS. 28a and 28b). The TER-119⁺ cells could be divided to Ki-67 high, low or negative subpopulations. In the NaOH-treated mice, the percentage of Ki-67^{hi} TER-119⁺ cells was higher than that of the saline-treated mice (76.2% vs 67%), whereas the percentage Ki-67^{lo} TER-119⁺ cells was lower (16% vs. 25.3%). On the other hand, the percentages of Ki-67^{neg} TER-119⁺ cells were similar (6.72% vs. 7.11%). These results indicated that the NaOH treatment had converted some of the Ki-67^{lo} TER-119⁺ cells to Ki-67^{hi} TER-119⁺ cells. (FIGS. 28c and 28d).

### Increase of Stem Cell Genesis in Bone Marrow by pH Modifiers

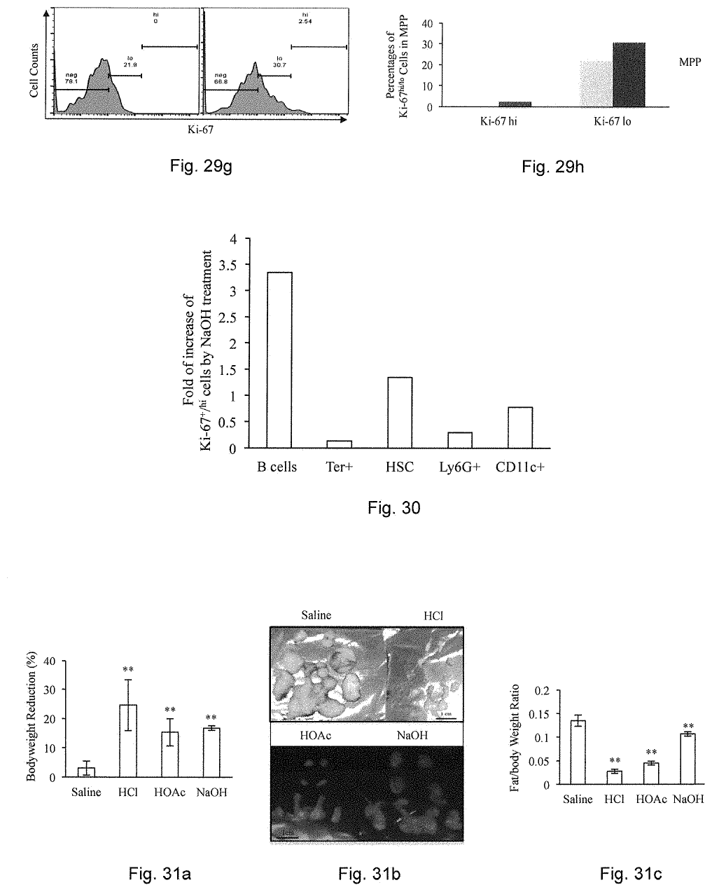

In the lineage negative (Lin⁻) WBM cells (stained negative for B220, CD3, CD11b, CD11c, Ly6G, CD48 and TER-119), Sca-1⁺ c-kit⁺ stem cell population was more than doubled in NaOH-treated mice as compared with that of the saline-treated mice (1.74% vs. 0.81%). (FIGS. 29a and 29b). Based on the expression of CD150, the Sca-1⁺ c-kit⁺ stem cells can be further categorized into hematopoietic stem cells (HSC) (CD150⁺) and their progenies multipotent progenitors (MPP) (CD150⁻). (108). These two categories of stem cells were present in both the saline- and NaOH-treated mice. (FIGS. 29c and 29d). In the NaOH-treated mice, the percentage of Ki-67^{hi} HSC increased by more than 1-fold as compared with that of the saline-treated mice (15.6% vs. 6.67%). (FIGS. 29e and 29f). Similarly, a distinct Ki-67^{hi} population of MPP (2.54%) was detected in the NaOH-treated mice, whereas this population was absent in the saline-treated mice (0%). Further, even the percentage of Ki-67^{lo} MPP was higher in the NaOH-treated mice than in the saline-treated mice (30.7% vs. 21.9%). (FIGS. 29g and 29h).

### Comparison of the Responsiveness of Different Lineages of WBM Cells to NaOH Treatments

The increases of the percentages of Ki-67^{+/hi} cells in the different bone marrow cell lineages by NaOH treatments were compared. (FIG. 30). The fold of increase is calculated as (% of NaOH-treated mice−% of saline-treated mice)/% of saline-treated mice. Ki-67⁺ B cells, Ly6G⁺ cells and CD11c⁺ cells, and Ki-67^{hi} TER-119⁺ cells and HSC are analyzed. The B cell lineage showed the highest increase, followed by the HSC, CD11c⁺ cells, Ly6G⁺ cells and Ter-119⁺ cells. (FIG. 30).

In summary, Example 9 shows that the pH modifiers such as NaOH that increase pH can enhance B cell lymphopoiesis, the genesis of all lineage cells and stem cells in the bone marrow. However, the degrees of the enhancement varied among different cell types/lineages.

### Example 10

### Control of Body Weight and Fat Mass by pH Modifiers

Like the thymus and bone marrow, the intestines maintain active cell proliferation in adults. The epithelial cells of the intestinal villi undergo constant renewal. It was therefore envisioned that the intestinal epithelial cell renewal/proliferation is amenable to manipulation by pH modifiers so that pH modifiers could be used to manage body weight and/or treatment of obesity.

### Reduction of Bodyweight and Fat Mass by In Vivo Treatments with pH Modifiers

Obesity was induced in C57BL/6 mice by feeding young (5 weeks old) female mice with high fat diet for 4 weeks. The mice were then switched to normal diet and randomly divided into groups to receive i.p. treatments with saline or saline plus HCl, HOAc or NaOH every other day for a total of four doses. The mice were analyzed 3 days after the final treatments. The saline-treated mice lost about 3% of body weight due to the change of diet; the HCl- and HOAc-treated mice lost about 25% and 15% of body weight, respectively, whereas the NaOH-treated mice lost about 17% of body weight. (FIG. 31a).

The fat pads of the mice were analyzed to further determine whether the bodyweight reductions were attributable to the reduction of fat masses. The sizes of fat pads of HCl- and HOAc-treated mice were grossly much smaller than those of the saline-treated mice. The sizes of fat pads of the NaOH-treated mice were also somewhat reduced. (FIG. 31b). Fat (weight of all fat pads)/bodyweight ratios of the mice were calculated to normalize the effects of individual variations of body weight on fat mass. The saline-treated mice had an average fat/bodyweight ratio of 0.135 (13.5%). The average fat/bodyweight ratios of the HCl- and HOAc-treated mice were reduced to just 0.027 (2.7%) and 0.045 (4.5%), representing 5- and 3-fold reduction, respectively. On the other hand, despite 17% of bodyweight reduction, the average fat/bodyweight ratio of the NaOH-treated mice was 0.106 (10.6%), modestly lower than that of the saline-treated mice. (FIG. 31c).

### Shortening of Intestinal Length by Treatments with pH Modifiers

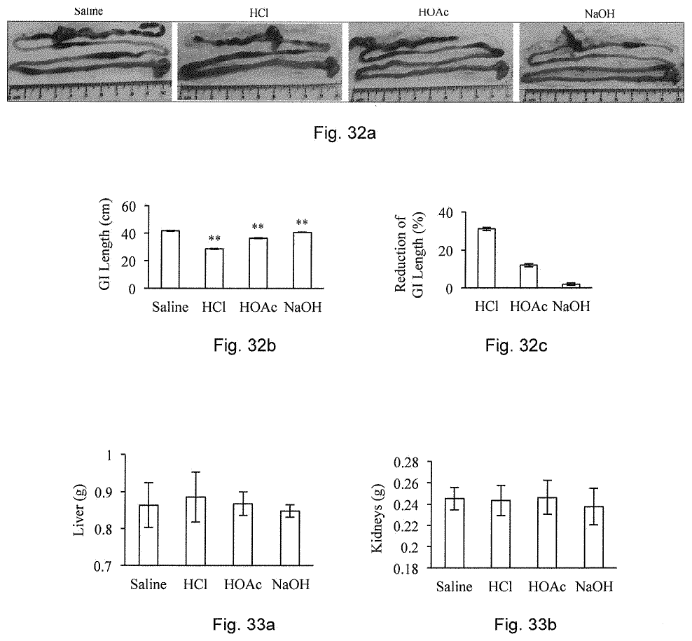

Gross examination of the entire intestines (from the end of stomach to anus) showed that the HCl- and HOAc-treated mice had drastically shortened intestines as compared with the saline-treated mice, whereas the intestines of the NaOH-treated mice were only slightly shortened. (FIG. 32a). Quantitative measurements found that the HCl- and HOAc-treated mice had on average about 31% and 12% reduction of intestinal lengths, respectively, whereas the NaOH-treated mice had only about 2% reduction. (FIGS. 32b and 32c). The reduction of the intestinal length by HCl or HOAc was likely due to the depletion of the proliferating cells and/or the inhibition of cell proliferation in the intestinal villi by the acid treatments as shown in Example 7.

### No Significant Reduction of Masses of Other Major Abdominal Organs

Despite the significant reduction of body weights and shortening of the intestines, the masses of the other two major abdominal organs the livers and kidneys were similar among the different treatment groups. (FIGS. 33a and 33b).

In summary, Example 10 shows that treatments with pH modifiers, particularly with acids, reduced body weights and fat masses. Acid treatments lead to the reduction of proliferating cells in the intestinal villi and shortening of the intestines. These effects varied considerably when different pH modifiers were administered to the experimental subjects. Among those three pH modifiers studied in this example, HCl exhibited the strongest effects on the reduction of body weight, fat mass and intestinal length. In contrast, although HOAc caused great reduction in body weight and fat mass, it caused only mild reduction of intestinal length.

### Example 11

### Treatment of Allergic Contact Dermatitis with pH Modifier

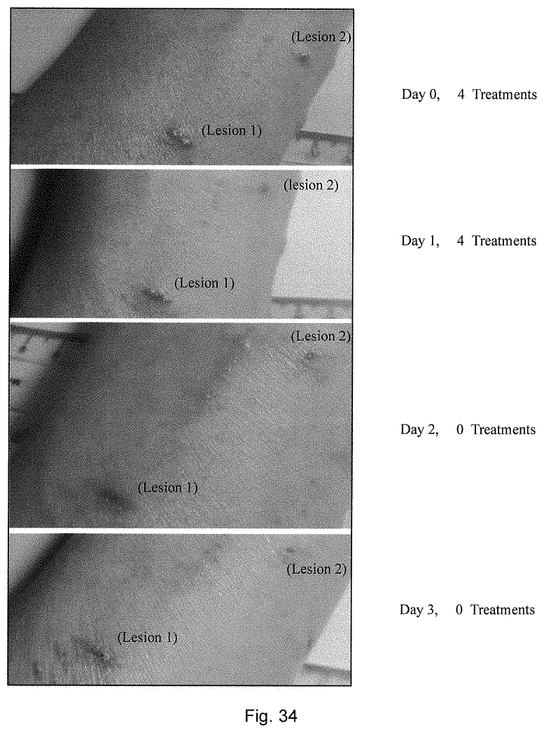

A male human subject developed allergic contact dermatitis after exposure to poison ivy. Two lesions on the left forearm (Lesion 1 and Lesion 2) were selected to receive topical application of a composition of about 5.25M HOAc in water, 4 times a day (about once every 4 hours) on day 0. On day 1, i.e., only 1 day after the first set of treatments, significant improvements were observed on both lesions. Specifically, rashes in lesion 1 had partially disappeared, and rash in lesion 2 had almost completely disappeared. On day 1, both lesions continued to receive the same treatments as on day 0. Although the treatments were discontinued after day 1, the lesions continue to improve. By day 2 and day 3, rashes in both lesions had completely disappeared, and were replaced by mild scars. (FIG. 34).

### REFERENCES

1. Fischer M, Ehlers M. 2008. Toll-like receptors in autoimmunity. Ann NY Acad Sci 1143:21-34.
2. Medzhitov R, Janeway C A, Jr. 1998. Innate immune recognition and control of adaptive immune responses. Semin Immunol 10:351-3.
3. Nagase H, Ueki S, Fujieda S. 2020. The roles of IL-5 and anti-IL-5 treatment in eosinophilic diseases: Asthma, eosinophilic granulomatosis with polyangiitis, and eosinophilic chronic rhinosinusitis. Allergol Int 69:178-186.
4. White M V. 1990. The role of histamine in allergic diseases. J Allergy Clin Immunol 86:599-605.
5. Tall A R, Yvan-Charvet L. 2015. Cholesterol, inflammation and innate immunity.

Nat Rev Immunol 15:104-16.
6. Tian S, Hu W, Niu L, Liu H, Xu H, Xiao S Y. 2020. Pulmonary Pathology of Early-Phase 2019 Novel Coronavirus (COVID-19) Pneumonia in Two Patients With Lung Cancer. J Thorac Oncol 15:700-704.
7. van Wijck Y, John-Schuster G, van Schadewijk A, van den Oever R L, Obieglo K, Hiemstra P S, Muller A, Smits H H, Taube C. 2019. Extract of Helicobacter pylori Ameliorates Parameters of Airway Inflammation and Goblet Cell Hyperplasia following Repeated Allergen Exposure. Int Arch Allergy Immunol 180:1-9.
8. Rogler G, Biedermann L, Scharl M. 2017. Anti-Cytokine Strategies beyond Anti-Tumour Necrosis Factor-alpha Therapy: Pathophysiology and Clinical Implications. Dig Dis 35:5-12.
9. Tayal V, Kalra B S. 2008. Cytokines and anti-cytokines as therapeutics—an update. Eur J Pharmacol 579:1-12.
10. Del Rosso J, Friedlander S F. 2005. Corticosteroids: options in the era of steroid-sparing therapy. J Am Acad Dermatol 53:S50-8.
11. Webster J M, Fenton C G, Langen R, Hardy R S. 2019. Exploring the Interface between Inflammatory and Therapeutic Glucocorticoid Induced Bone and Muscle Loss. Int J Mol Sci 20.
12. Ricciotti E, FitzGerald G A. 2011. Prostaglandins and inflammation. Arterioscler Thromb Vasc Biol 31:986-1000.
13. Leung D Y, Bloom J W. 2003. Update on glucocorticoid action and resistance. J Allergy Clin Immunol 111:3-22; quiz 23.
14. Barnes P J. 2013. Corticosteroid resistance in patients with asthma and chronic obstructive pulmonary disease. J Allergy Clin Immunol 131:636-45.
15. Yang I A, Clarke M S, Sim E H, Fong K M. 2012. Inhaled corticosteroids for stable chronic obstructive pulmonary disease. Cochrane Database Syst Rev doi:10.1002/14651858.CD002991.pub3:CD002991.
16. Berthon B S, Gibson P G, Wood L G, MacDonald-Wicks L K, Baines K J. 2017. A sputum gene expression signature predicts oral corticosteroid response in asthma. Eur Respir J 49.
17. Dunican E M, Fahy J V. 2017. Asthma and corticosteroids: time for a more precise approach to treatment. Eur Respir J 49.
18. Peters M C, Kerr S, Dunican E M, Woodruff P G, Fajt M L, Levy B D, Israel E, Phillips B R, Mauger D T, Comhair S A, Erzurum S C, Johansson M W, Jarjour N N, Coverstone A M, Castro M, Hastie A T, Bleecker E R, Wenzel S E, Fahy J V, National Heart L, Blood Institute Severe Asthma Research P. 2019. Refractory airway type 2 inflammation in a large subgroup of asthmatic patients treated with inhaled corticosteroids. J Allergy Clin Immunol 143:104-113 e14.
19. Szefler S J, Martin R J, King T S, Boushey H A, Cherniack R M, Chinchilli V M, Craig T J, Dolovich M, Drazen J M, Fagan J K, Fahy J V, Fish J E, Ford J G, Israel E, Kiley J, Kraft M, Lazarus S C, Lemanske R F, Jr., Mauger E, Peters S P, Sorkness C A, Asthma Clinical Research Network of the National Heart L, Blood I. 2002. Significant variability in response to inhaled corticosteroids for persistent asthma. J Allergy Clin Immunol 109:410-8.
20. Woodruff P G, Modrek B, Choy D F, Jia G, Abbas A R, Ellwanger A, Koth L L, Arron J R, Fahy J V. 2009. T-helper type 2-driven inflammation defines major subphenotypes of asthma. American journal of respiratory and critical care medicine 180:388-95.
21. Oladosu F A, Tu F F, Hellman K M. 2018. Nonsteroidal antiinflammatory drug resistance in dysmenorrhea: epidemiology, causes, and treatment. Am J Obstet Gynecol 218:390-400.
22. Green G A. 2001. Understanding NSAIDs: from aspirin to COX-2. Clin Cornerstone 3:50-60.
23. Shiri R, Koskimaki J, Hakkinen J, Tammela T L, Auvinen A, Hakama M. 2006. Effect of nonsteroidal anti-inflammatory drug use on the incidence of erectile dysfunction. J Urol 175:1812-5; discussion 1815-6.
24. Rodgers G M. 2012. A perspective on the evolution of management of cancer- and chemotherapy-induced anemia. J Natl Compr Canc Netw 10:434-7.
25. Luqmani Y A. 2005. Mechanisms of drug resistance in cancer chemotherapy. Med Princ Pract 14 Suppl 1:35-48.
26. Heymsfield S B, Wadden T A. 2017. Mechanisms, Pathophysiology, and Management of Obesity. N Engl J Med 376:1492.
27. Hopkins E, Sharma S. 2019. Acid Base Balance, Physiology. StatPearls Publishing, NCBI Bookshelf.
28. Bangsbo J, Johansen L, Graham T, Saltin B. 1993. Lactate and H+ effluxes from human skeletal muscles during intense, dynamic exercise. J Physiol 462:115-33.
29. Orringer C E, Eustace J C, Wunsch C D, Gardner L B. 1977. Natural history of lactic acidosis after grand-mal seizures. A model for the study of an anion-gap acidosis not associated with hyperkalemia. N Engl J Med 297:796-9.
30. Bodem C R, Lampton L M, Miller D P, Tarka E F, Everett E D. 1983. Endobronchial pH. Relevance of aminoglycoside activity in gram-negative bacillary pneumonia. Am Rev Respir Dis 127:39-41.
31. Nielson D W, Goerke J, Clements J A. 1981. Alveolar subphase pH in the lungs of anesthetized rabbits. Proc Natl Acad Sci USA 78:7119-23.
32. Bushinsky D. 2020. Kidney stones. In Melmed S, Auchus R, Goldfine A, Koenig R, Rosen C (ed), Williams Textbook of Endocrinology, 14th ed. Elsevier, Philadelphia, Pa.
33. Berdis A J. 2017. Inhibiting DNA Polymerases as a Therapeutic Intervention against Cancer. Front Mol Biosci 4:78.
34. Duffy M J, O'Grady S, Tang M, Crown J. 2021. MYC as a target for cancer treatment. Cancer Treat Rev 94:102154.
35. Ingham M, Schwartz G K. 2017. Cell-Cycle Therapeutics Come of Age. J Clin Oncol 35:2949-2959.
36. Madden S K, de Araujo A D, Gerhardt M, Fairlie D P, Mason J M. 2021. Taking the Myc out of cancer: toward therapeutic strategies to directly inhibit c-Myc. Mol Cancer 20:3.
37. Parker W B. 2009. Enzymology of purine and pyrimidine antimetabolites used in the treatment of cancer. Chem Rev 109:2880-93.
38. Qin Q, Ren Y, Zhong D. 2020. [Research Progress of CDK4/6 Inhibitors in Non-small Cell Lung Cancer]. Zhongguo Fei Ai Za Zhi 23:176-181.
39. Ballweg R, Lee S, Han X, Maini P K, Byrne H, Hong C I, Zhang T. 2018. Unraveling the Control of Cell Cycle Periods during Intestinal Stem Cell Differentiation. Biophys J 115:2250-2258.
40. Merenda A, Fenderico N, Maurice M M. 2020. Wnt Signaling in 3D: Recent Advances in the Applications of Intestinal Organoids. Trends Cell Biol 30:60-73.
41. Pinto D, Gregorieff A, Begthel H, Clevers H. 2003. Canonical Wnt signals are essential for homeostasis of the intestinal epithelium. Genes Dev 17:1709-13.
42. Suzuki A, Sekiya S, Gunshima E, Fujii S, Taniguchi H. 2010. EGF signaling activates proliferation and blocks apoptosis of mouse and human intestinal stem/progenitor cells in long-term monolayer cell culture. Lab Invest 90:1425-36.
43. Harb J, Lin P J, Hao J. 2019. Recent Development of Wnt Signaling Pathway Inhibitors for Cancer Therapeutics. Curr Oncol Rep 21:12.
44. Xu M J, Johnson D E, Grandis J R. 2017. EGFR-targeted therapies in the post-genomic era. Cancer Metastasis Rev 36:463-473.
45. Zhong F-F, You Y, Liu W-J. 2021. Progress in research on childhood T-cell acute lymphocytic leukemia, Notch1 signaling pathway, and its inhibitors: A review. Bosn J Basic Med Sci 21:136-144.
46. Weaver C T, Harrington L E, Mangan P R, Gavrieli M, Murphy K M. 2006. Th17: an effector CD4 T cell lineage with regulatory T cell ties. Immunity 24:677-88.
47. Itoh M, Takahashi T, Sakaguchi N, Kuniyasu Y, Shimizu J, Otsuka F, Sakaguchi S. 1999. Thymus and autoimmunity: production of CD25+CD4+ naturally anergic and suppressive T cells as a key function of the thymus in maintaining immunologic self-tolerance. J Immunol 162:5317-26.
48. Chen W, Jin W, Hardegen N, Lei K J, Li L, Marinos N, McGrady G, Wahl S M. 2003. Conversion of peripheral CD4+CD25− naïve T cells to CD4+CD25+ regulatory T cells by TGF-beta induction of transcription factor Foxp3. J Exp Med 198:1875-86.
49. Matsushita T. 2019. Regulatory and effector B cells: Friends or foes? J Dermatol Sci 93:2-7.
50. Vivier E, Artis D, Colonna M, Diefenbach A, Di Santo J P, Eberl G, Koyasu S, Locksley R M, McKenzie A N J, Mebius R E, Powrie F, Spits H. 2018. Innate Lymphoid Cells: 10 Years On. Cell 174:1054-1066.
51. Ricketts T D, Prieto-Dominguez N, Gowda P S, Ubil E. 2021. Mechanisms of Macrophage Plasticity in the Tumor Environment: Manipulating Activation State to Improve Outcomes. Front Immunol 12:642285.
52. Gregori S, Goudy K S, Roncarolo M G. 2012. The cellular and molecular mechanisms of immuno-suppression by human type 1 regulatory T cells. Front Immunol 3:30.
53. Dou A, Fang J. 2021. Heterogeneous Myeloid Cells in Tumors. Cancers (Basel) 13.
54. Benassi A, Doria F, Pirota V. 2020. Groundbreaking Anticancer Activity of Highly Diversified Oxadiazole Scaffolds. Int J Mol Sci 21.
55. Ch'en F F, Villafuerte F C, Swietach P, Cobden P M, Vaughan-Jones R D. 2008. S0859, an N-cyanosulphonamide inhibitor of sodium-bicarbonate cotransport in the heart. Br J Pharmacol 153:972-82.
56. Felmlee M A, Jones R S, Rodriguez-Cruz V, Follman K E, Morris M E. 2020. Monocarboxylate Transporters (SLC16): Function, Regulation, and Role in Health and Disease. Pharmacol Rev 72:466-485.
57. Lugini L, Federici C, Borghi M, Azzarito T, Marino M L, Cesolini A, Spugnini E P, Fais S. 2016. Proton pump inhibitors while belonging to the same family of generic drugs show different anti-tumor effect. J Enzyme Inhib Med Chem 31:538-45.
58. Meleddu R, Deplano S, Maccioni E, Ortuso F, Cottiglia F, Secci D, Onali A, Sanna E, Angeli A, Angius R, Alcaro S, Supuran C T, Distinto S. 2021. Selective inhibition of carbonic anhydrase IX and XII by coumarin and psoralen derivatives. J Enzyme Inhib Med Chem 36:685-692.
59. Mihaila R G. 2015. A minireview on NHE1 inhibitors. A rediscovered hope in oncohematology. Biomed Pap Med Fac Univ Palacky Olomouc Czech Repub 159:519-26.
60. Scozzafava A, Supuran C T. 2002. Carbonic anhydrase activators: human isozyme II is strongly activated by oligopeptides incorporating the carboxyterminal sequence of the bicarbonate anion exchanger AE1. Bioorg Med Chem Lett 12:1177-80.
61. Temperini C, Scozzafava A, Vullo D, Supuran C T. 2006. Carbonic anhydrase activators. Activation of isozymes I, II, IV, VA, VII, and XIV with l- and d-histidine and crystallographic analysis of their adducts with isoform II: engineering proton-transfer processes within the active site of an enzyme. Chemistry 12:7057-66.
62. Whitton B, Okamoto H, Packham G, Crabb S J. 2018. Vacuolar ATPase as a potential therapeutic target and mediator of treatment resistance in cancer. Cancer Med 7:3800-3811.
63. Bulli I, Dettori I, Coppi E, Cherchi F, Venturini M, Di Cesare Mannelli L, Ghelardini C, Nocentini A, Supuran C T, Pugliese A M, Pedata F. 2021. Role of Carbonic Anhydrase in Cerebral Ischemia and Carbonic Anhydrase Inhibitors as Putative Protective Agents. Int J Mol Sci 22.
64. Perry S F, Purohit A M, Boser S, Mitchell I, Green F H. 2000. Bronchial casts of human lungs using negative pressure injection. Exp Lung Res 26:27-39.
65. Boucher R C. 2019. Muco-Obstructive Lung Diseases. N Engl J Med 380:1941-1953.
66. Lechner K, von Schacky C, McKenzie A L, Worm N, Nixdorff U, Lechner B, Krankel N, Halle M, Krauss R M, Scherr J. 2020. Lifestyle factors and high-risk atherosclerosis: Pathways and mechanisms beyond traditional risk factors. Eur J Prev Cardiol 27:394-406.
67. Mozaffarian D, Benjamin E J, Go A S, Arnett D K, Blaha M J, Cushman M, de Ferranti S, Despres J P, Fullerton H J, Howard V J, Huffman M D, Judd S E, Kissela B M, Lackland D T, Lichtman J H, Lisabeth L D, Liu S, Mackey R H, Matchar D B, McGuire D K, Mohler E R, 3rd, Moy C S, Muntner P, Mussolino M E, Nasir K, Neumar R W, Nichol G, Palaniappan L, Pandey D K, Reeves M J, Rodriguez C J, Sorlie P D, Stein J, Towfighi A, Turan T N, Virani S S, Willey J Z, Woo D, Yeh R W, Turner M B, American Heart Association Statistics C, Stroke Statistics S. 2015. Heart disease and stroke statistics—2015 update: a report from the American Heart Association. Circulation 131:e29-322.
68. Rahman M S, Murphy A J, Woollard K J. 2017. Effects of dyslipidaemia on monocyte production and function in cardiovascular disease. Nat Rev Cardiol 14:387-400.
69. Rahman M S, Woollard K. 2017. Atherosclerosis. Adv Exp Med Biol 1003:121-144.
70. Klineova S, Lublin F D. 2018. Clinical Course of Multiple Sclerosis. Cold Spring Harb Perspect Med 8.
71. Okuda D T, Siva A, Kantarci O, Inglese M, Katz I, Tutuncu M, Keegan B M, Donlon S, Hua le H, Vidal-Jordana A, Montalban X, Rovira A, Tintore M, Amato M P, Brochet B, de Seze J, Brassat D, Vermersch P, De Stefano N, Sormani M P, Pelletier D, Lebrun C, Radiologically Isolated Syndrome C, Club Francophone de la Sclerose en P. 2014. Radiologically isolated syndrome: 5-year risk for an initial clinical event. PLoS One 9:e90509.
72. Domoslawski P, Podhorska-Okolow M, Pula B, Lukienczuk T, Dziegiel P. 2013. Expression of estrogen and progesterone receptors and Ki-67 antigen in Graves' disease and nodular goiter. Folia Histochem Cytobiol 51:135-40.
73. Potter M, Newport E, Morten K J. 2016. The Warburg effect: 80 years on. Biochem Soc Trans 44:1499-1505.
74. Warburg O. 1923. Versuche an iberlebendem Carcinomgewebe. Biochem Zschr 142:317-333.
75. Pfeiffer T, Morley A. 2014. An evolutionary perspective on the Crabtree effect. Front Mol Biosci 1:17.
76. Ziv N, Brandt N J, Gresham D. 2013. The use of chemostats in microbial systems biology. J Vis Exp doi:10.3791/50168.
77. Homewood C A. 1977. Carbohydrate metabolism of malarial parasites. Bull World Health Organ 55:229-35.
78. Moyersoen J, Choe J, Fan E, Hol W G, Michels P A. 2004. Biogenesis of peroxisomes and glycosomes: trypanosomatid glycosome assembly is a promising new drug target. FEMS Microbiol Rev 28:603-43.
79. You H, Stephenson R J, Gobert G N, McManus D P. 2014. Revisiting glucose uptake and metabolism in schistosomes: new molecular insights for improved schistosomiasis therapies. Front Genet 5:176.
80. Keenleyside W. 2019. The Effects of pH on Microbial Growth In Keenleyside W (ed), Microbiology: Canadian Edition. Pressbooks, https://ecampusontario.pressbooks.pub/microbio.
81. Jin Q, Kirk M. 2018. pH as a Primary Control in Environmental Microbiology: 1. Thermodynamic Perspective. Front Envir Sci 6:Article 21.
82. Dyson N, Buchkovich K, Whyte P, Harlow E. 1989. The cellular 107K protein that binds to adenovirus E1A also associates with the large T antigens of SV40 and J C virus. Cell 58:249-55.
83. Fan Y, Sanyal S, Bruzzone R. 2018. Breaking Bad: How Viruses Subvert the Cell Cycle. Front Cell Infect Microbiol 8:396.
84. Knight J S, Robertson E S. 2004. Epstein-Barr virus nuclear antigen 3C regulates cyclin A/p27 complexes and enhances cyclin A-dependent kinase activity. J Virol 78:1981-91.
85. Lehman J M, Laffin J, Friedrich T D. 2000. Simian virus 40 induces multiple S phases with the majority of viral DNA replication in the G2 and second S phase in CV-1 cells. Exp Cell Res 258:215-22.
86. Moore P S, Chang Y. 1998. Kaposi's sarcoma-associated herpesvirus-encoded oncogenes and oncogenesis. J Natl Cancer Inst Monogr doi:10.1093/oxfordjournals.jncimonographs.a024176:65-71.
87. Corbet C, Feron O. 2017. Cancer cell metabolism and mitochondria: Nutrient plasticity for TCA cycle fueling. Biochim Biophys Acta Rev Cancer 1868:7-15.
88. Fenton T R, Huang T. 2016. Systematic review of the association between dietary acid load, alkaline water and cancer. BMJ Open 6:e010438.
89. Pravetoni M, Comer S D. 2019. Development of vaccines to treat opioid use disorders and reduce incidence of overdose. Neuropharmacology 158:107662.
90. Wolfson B, Franks S E, Hodge J W. 2021. Stay on Target: Reengaging Cancer Vaccines in Combination Immunotherapy. Vaccines (Basel) 9.
91. Aksan A, Farrag K, Blumenstein I, Schroder O, Dignass A U, Stein J. 2021. Chronic intestinal failure and short bowel syndrome in Crohn's disease. World J Gastroenterol 27:3440-3465.
92. Rangasamy T, Guo J, Mitzner W A, Roman J, Singh A, Fryer A D, Yamamoto M, Kensler T W, Tuder R M, Georas S N, Biswal S. 2005. Disruption of Nrf2 enhances susceptibility to severe airway inflammation and asthma in mice. J Exp Med 202:47-59.
93. Zeng W P, McFarland M M, Zhou B, Holtfreter S, Flesher S, Cheung A, Mallick A. 2017. Staphylococcal enterotoxin A-activated regulatory T cells promote allergen-specific TH2 response to intratracheal allergen inoculation. J Allergy Clin Immunol 139:508-518 e4.
94. Zeng W P, Chang C, Lai J J. 2009. Immune suppressive activity and lack of T helper differentiation are differentially regulated in natural regulatory T cells. J Immunol 183:3583-90.
95. Incio J, Liu H, Suboj P, Chin S M, Chen I X, Pinter M, Ng M R, Nia H T, Grahovac J, Kao S, Babykutty S, Huang Y, Jung K, Rahbari N N, Han X, Chauhan V P, Martin J D, Kahn J, Huang P, Desphande V, Michaelson J, Michelakos T P, Ferrone C R, Soares R, Boucher Y, Fukumura D, Jain R K. 2016. Obesity-Induced Inflammation and Desmoplasia Promote Pancreatic Cancer Progression and Resistance to Chemotherapy. Cancer Discov 6:852-69.
96. Martinez-Huenchullan S F, McLennan S V, Ban L A, Morsch M, Twigg S M, Tam C S. 2017. Utility and reliability of non-invasive muscle function tests in high-fat-fed mice. Exp Physiol 102:773-778.
97. Crosby K, Standen C. 2014. Immunohistochemistry Protocol for Paraffin-embedded Tissue Sections. JOVE doi:10.3791/5064.
98. Jutel M, Akdis C A. 2011. T-cell subset regulation in atopy. Current allergy and asthma reports 11:139-45.
99. Arpaia N, Campbell C, Fan X, Dikiy S, van der Veeken J, deRoos P, Liu H, Cross J R, Pfeffer K, Coffer P J, Rudensky A Y. 2013. Metabolites produced by commensal bacteria promote peripheral regulatory T-cell generation. Nature 504:451-5.
100. Smith P M, Howitt M R, Panikov N, Michaud M, Gallini C A, Bohlooly Y M, Glickman J N, Garrett W S. 2013. The microbial metabolites, short-chain fatty acids, regulate colonic Treg cell homeostasis. Science 341:569-73.
101. Lee P Y, Wang J X, Parisini E, Dascher C C, Nigrovic P A. 2013. Ly6 family proteins in neutrophil biology. J Leukoc Biol 94:585-94.
102. Misharin A V, Morales-Nebreda L, Mutlu G M, Budinger G R, Perlman H. 2013. Flow cytometric analysis of macrophages and dendritic cell subsets in the mouse lung. Am J Respir Cell Mol Biol 49:503-10.
103. Darzynkiewicz Z, Zhao H, Zhang S, Lee M Y, Lee E Y, Zhang Z. 2015. Initiation and termination of DNA replication during S phase in relation to cyclins D1, E and A, p21WAF1, Cdt1 and the p12 subunit of DNA polymerase delta revealed in individual cells by cytometry. Oncotarget 6:11735-50.
104. Harkness J, Wagner J. 1989. Biology and husbandry, p 372. In Harkness J, Wagner J (ed), The biology and medicine of rabbits and rodents, 3rd ed. Lea & Febiger, Philadelphia.
105. Mitruka B, Rawnsley H. 1981. Clinical, biochemical and hematological reference values in normal experimental animals and normal humans. Masson Publishing, New York.
106. Fornetti J, Welm A L, Stewart S A. 2018. Understanding the Bone in Cancer Metastasis. J Bone Miner Res 33:2099-2113.
107. Exner G U, von Hochstetter A R, Honegger H, Schreiber A. 2000. Osseous lesions of the distal ulna: atypical location—unusual diagnosis. Report of three cases with similar imaging and different pathologic diagnoses. Arch Orthop Trauma Surg 120:219-23.
108. Acar M, Kocherlakota K S, Murphy M M, Peyer J G, Oguro H, Inra C N, Jaiyeola C, Zhao Z, Luby-Phelps K, Morrison S J. 2015. Deep imaging of bone marrow shows non-dividing stem cells are mainly perisinusoidal. Nature 526:126-30.
109. Altorki N K, Markowitz G J, Gao D, Port J L, Saxena A, Stiles B, McGraw T, Mittal V. 2019. The lung microenvironment: an important regulator of tumour growth and metastasis. Nat Rev Cancer 19:9-31.
110. NCI. 2011. Metastatic Cancer, http://www.cancer.gov/cancertopics/factsheet/Sites-Types/metastatic. National Cancer Institute, Rockville.
111. Barsouk A, Rawla P, Barsouk A, Thandra K C. 2019. Epidemiology of Cancers of the Small Intestine: Trends, Risk Factors, and Prevention. Med Sci (Basel) 7.
112. CSC. 2021. https://cancerstatisticscenter.cancer.org, American Cancer Society.
113. Qualai J, Li L X, Cantero J, Tarrats A, Fernandez M A, Sumoy L, Rodolosse A, McSorley S J, Genesca M. 2016. Expression of CD11c Is Associated with Unconventional Activated T Cell Subsets with High Migratory Potential. PLoS One 11:e0154253.
114. Expression-Atlas. 2021. www.ebi.ac.uk/gxa/genes/ensmusg0000022582.
115. Kina T, Ikuta K, Takayama E, Wada K, Majumdar A S, Weissman I L, Katsura Y. 2000. The monoclonal antibody TER-119 recognizes a molecule associated with glycophorin A and specifically marks the late stages of murine erythroid lineage. Br J Haematol 109:280-7.

What is claimed is:

1. A method for selectively depleting, selectively suppressing, or both selectively deleting and selectively suppressing the population, proliferation or both the population and proliferation of pathological cells in a subject in need of treatment of, prevention from, or both treatment of and prevention from a disease with a pathogenesis that is partially or fully attributable to inflammation, immune response cell proliferation, or combinations thereof; the method comprising either:
administering a composition comprising one or more pH modifiers that decrease pH, resist the rise of pH, or both to the subject; or
administering a composition comprising one or more pH modifiers that increase pH, resist the fall of pH, or both to the subject.

2. The method of claim 1, further comprising:
choosing the one or more pH modifiers, and routes and means of administration;
titrating doses, dosing regimens, or both the doses and dosing regimens of the one or more pH modifiers, and titrating the concentrations of the one or more pH modifiers in the composition; or
both the said choosing and titrating so that the pH modifiers selectively act on pathological cells.

3. The method of claim 1, wherein the disease is selected from: one or more allergic diseases; autoimmune diseases; infectious diseases; inflammatory diseases of the blood, blood vessels, or both; diseases with pulmonary inflammation; muco-obstructive lung diseases; and other diseases sharing the characteristic of an overzealous inflammatory response, immune response, or both that contribute to pathogenesis,
wherein the step of administrating dampens the overzealous inflammatory response, immune response, or both; and
wherein the pathological cells comprise effector inflammatory cells; hyperplastic, hyperactive structural functional cells of diseased tissues or organs; host target cells; or combinations thereof.

4. The method of claim 1, wherein the disease is selected from one or more of neoplastic diseases and infectious diseases where lacking or insufficiency of protective inflammatory responses, immune responses, or both against neoplastic cells or infectious agents contributes to pathogenesis;
wherein the step of administering promotes the protective inflammatory responses, immune responses, or both; and clearance of neoplastic cells or infections; and
wherein the pathological cells comprise neoplastic cells; functional subsets of inflammatory cells with anti-inflammatory, immune suppressive activities, or both; non-inflammatory tumor-promoting cells; hyperplastic, hyperactive structural functional cells of diseased tissues or organs; host target cells, or combinations thereof.

5. The method of claim 1, wherein the step of administering suppresses mucus hypersecretion in asthma or muco-obstructive lung diseases where mucus hypersecretion obstructs airflow, impairs respiratory function, or both.

6. The method of claim 1, wherein the step of administering the pH modifier(s) depletes, suppresses, or both depletes and suppresses population, proliferation, or both the population and proliferation, and dissemination of infectious agents in or on the body of the subject.

7. The method of claim 1, wherein the subject is a recipient of vaccination, the step of administering selectively depletes and suppresses a population, proliferation, or both the population and proliferation of one or more of functional subsets of inflammatory cells that undermine efficacy or protective effects of the vaccination against one or more diseases selected from infectious diseases, neoplastic diseases, allergic diseases, and drug addictions.

8. A method for selectively increasing population, proliferation, or both the population and proliferation of protective normal cells against a disease in a subject in need of treatment of, prevention from, or both treatment of and prevention from the disease with a pathogenesis that is partially or fully attributable to inflammation, immune response, cell proliferation, or combinations thereof; the method comprising:
administering a composition comprising one or more pH modifiers that increase pH, resist the fall of pH, or both to the subject.

9. The method of claim 8, further comprising choosing the one or more pH modifiers, and routes and means of administration; titrating doses, dosing regimens, or both doses and dosing regimens of the one or more pH modifiers, and titrating the concentrations of the one or more pH modifiers in the composition; or both the said choosing and titrating to achieve the selective effect of the pH modifiers on the protective normal cells.

10. The method of claim 8, wherein the disease is selected from one or more of neoplastic diseases and infectious diseases where lacking or insufficiency of protective inflammatory responses, immune responses, or both against neoplastic cells or infectious agents contributes to pathogenesis;
wherein the step of administering promotes the protective inflammatory response, immune response, or both; clearance of neoplastic cells or infections; repair and regeneration of damaged tissues or organs; or combinations thereof; and
wherein the protective normal cells comprise effector inflammatory cells that are directly or indirectly reactive to the neoplastic cells or infectious agents, and precursors of the protective inflammatory cells; structural functional cells of diseased tissues or organs, and their precursors; or combinations thereof.

11. The method of claim 8, wherein the disease is selected from one or more allergic diseases; autoimmune diseases; infectious diseases; diseases of pulmonary inflammation; muco-obstructive lung diseases; inflammatory diseases of blood, blood vessels, or both; and other diseases sharing the characteristic of overzealous inflammatory responses, immune responses, or both that contribute to pathogenesis;
wherein the step of administering dampens the overzealous inflammatory responses, immune responses, or both; and promotes restoration of damaged tissues or organs; and
wherein the protective normal cells comprise functional subsets of inflammatory cells with anti-inflammatory, immune suppressive activities, or both, and their precursors; structural functional cells of tissues or organs, and their precursors; or combinations thereof.

12. The method of claim 8, wherein the subject is a recipient of vaccination, the step of administering enhances the efficacy or protective effects of the vaccination against one or more diseases selected from infectious diseases, neoplastic diseases, allergic diseases, and drug addictions, and wherein the protective normal cells are one or more functional subsets of inflammatory cells directly or indirectly reactive to infectious agents, neoplastic cells, or one or more addictive drugs or molecules involved in the drugs' actions.

13. The method of claim 8, wherein the subject is an autologous or allogeneic donor of blood, bone marrow, or stem cells for adoptive cell transfer; or suffers from anemia or insufficient genesis of lymphocytes and hematopoietic cells; and wherein the step of administering increases the population, proliferation, or both the population and proliferation of lymphocytes, hematopoietic cells, precursors of lymphocytes and hematopoietic cells; other stem cells; or combinations thereof.

14. The method of claim 8, wherein the subject suffers from one or more wounds, and wherein the step of administering facilitates wound healing.

15. The method of claim 8, wherein the step of administering the pH modifier(s) depletes, suppresses, or both depletes and suppresses population, proliferation, or both the population and proliferation; and dissemination of infectious agents in or on the body of the subject.

16. A method of reducing body weight, fat mass, or both body weight and fat mass in a subject who suffers from overweight or obesity, the method comprising either:
administering a composition to the subject, wherein the step of administering selectively reduces population, proliferation, or both the population and proliferation of proliferating cells in epithelium of intestinal villi of the subject; or
surgical removal of a segment of small intestines of the subject;
wherein the composition comprises one or more pH modifiers, cell proliferation inhibitors, or both; and
wherein the step of administering further comprises choosing the one or more of pH modifiers or cell proliferation inhibitors, or both, and routes and means of administration; titrating doses, dosing regimens, or both the doses and dosing regimens of the one or more of pH modifiers or cell proliferation inhibitors, and titrating the concentrations of the one or more of pH modifiers or cell proliferation inhibitors in the composition; or both the said choosing and titrating to achieve the selective effects on proliferating cells in the epithelium of the intestinal villi of the subject.
